SEQUENCE LISTING (17 sequences)

SEQ ID NO: 1 - DNA, 769 bp, Homo sapiens
tatattatagacatatgttagaaaagtcctagaaatgcacccaatttccttccattttactttcctacatggattgaagtcagcccctcaaaagcttttcggctgggcatggtggttcacgcccataatactagcactttgggaggccaaggtgggtggatcaactgaggtcaggaattcaagaccagcctggccaagatggtgaaaccccatctctactaaaaaatacaaaaattagcttggtgtggtagtgcgcacctgtaatcccagctactcgggaggctgaggcagacaattgcttgaacccgagagacggagggtgcagtgagccgagatcgtgctactgcactccagcctgggcaacagagcaagactccgtctcaaaaaaaaaaaaaaaaaaaaagcttttcaaaagtccacccaggattttttaagacattttcccatttgtttttgcttggacgacctggttactccttgagtgcggaatatataatctaaagcatgttatgtgcaaaaggtgccatggtgttaaaaattaaaacttggaattggttgtagtaccattygttacatttcaaagtgactaatgctgatgtcaaaaccagaatgctaatggtaataacaggtcatatcaagagttttttgaactccagggtcttcatgctccgaaatactcatttgcgtttccatgtattctatcctcttaaaagttctcggtcacagtgcaatatagcatttgcagtagctccaacagtgcttcctttat

SEQ ID NO: 2 - DNA, 601 bp, Homo sapiens
gtgtgctttgggtcatgatagattaattaatctcatctaaacattgatgtctttttctgttgctgtctagactgtgaacaatgtctaacaccttagggaagaggtggggaggaatcccaatgtatacattgcccttaagcagtgtttgattcattcatctttggactccatgaatcgaaatctggtagaatacatgatcttagtggaggaggccaaatgcgtgactcactgagcctggcagagcagaaatactctgctgtctgcaccctctgggtctggtgtggctctgcttcttggtgcytcaactctgactggcagctgtccccaggaggcgataattcagcatgttcaatctaaaggttatgacttccttgatggttttcaccatattcttggcaagtttttggtttttgaaatgttctaggaggcttggtagagatcttatgaaatagagaatagctgctgtggaaattattttaatgctaattacataaaagtacaaaagtagcactagctaaaacaaaaggtattttgctgttctgttttgttttagcttgtgccaggccttttacagcattaggaatgcaacttctagataacg

SEQ ID NO: 3 - DNA, 601 bp, Homo sapiens
actgctaagcttgactttactgacaggagtaaaaaaaattgtgttaaggttagggaataattttaacagtcaatttgttcttgtgaacaaatttcaacagtgaaattttagatatgtactttttaatggtgccaagcagcagttattatagatcaactgctgtttggcaccattaaaaagtacacttcgcaccgtcaaaaagtagatctggccacaattagatcagtcagggaaaaacacttcgcaatgaaatattatttaccacgttttcttcctccctcttcttgaaaatagtaatgaytttagcatttttaaatcttgaagaatgtcattccgtactgactaaaaagcctgtgcaaacacccaacatcttctctttcctgtctattttaatggatatacaaaataaatattcatctaatttatcaatatttaaggcacattgtgtatcacaatccaatggtaaacatttttcttatcaaatggctgatcttgacattgtttaaagccctatatgatttcagaaaagtcagtgactccaagtggaattgcaacaatttcttgggtcttgtaacaaacctgaagtgtaactatttctctt

SEQ ID NO: 4 - DNA, 511 bp, Homo sapiens
tgtcttcttcaatgtgggaaccatcctttctggaaccacagagctgcagatgtcacgctggaattctcccagaggcaaactgccagatgtgaggctgctcttcctcagtcactatctctggtcgtaccgggcgatgcctgagccaactgaccctcagacctgtgagccgagccggtcacaccgtggctgacaccggcattcccaccgcctttctcctgtgcgacccgctaagggccccgcgaggtgggcaggccargtattcttgaccttcgtggggtagaagaagccaccgtggctgggagagggccctgctcacagccacacgtttacttcgctgcaggtcccgagcttctgccccaggtgggcaaagcatccgggaaatgccctccgctgcccgaggggagcccagagcccgtgctttctattaaatgttgtaaatgccgcctctcccactttatcaccaaatggaagggaagaattcttccaaggcgccctccctttcctgccatag

SEQ ID NO: 5 - DNA, 511 bp, Homo sapiens
ttactctatattgcctgcaagacacgactggagaattcttagtgtaagaattgtttgtttcctctctccttctttcccacgttttcccagggaagtcagtcttgcattttaatgcatactatatacatatctcgtttagcttactgaaccacttgttttaacagaataaaactgtgcaaaattttaattttcctcctttgcctgaactgaaatagcacatccaggtttagcccttgtagactttccttcctcgaarcagaaagttgcccttgatgatttcctctttgagctctctgccagctctgaaacccacaaaatttatgtttgcaaaactaagccatgcaatcctctttttatgcaggctctagcctgagtcattttccctaagagatcttcagctccacctgggatgtgattctttgctctctgggattgaaggtagctgaagagaaatagttacacttcaggtttgttacaagacccaagaaattgttgcaattccacttggagt

SEQ ID NO: 6 - DNA, 711 bp, Homo sapiens
ttcagatttcaccaattgagaattagtaagtaatttctctgatacaggcctgaagtttaccttagtaaacactttacttccatatggtaaaaattagattttgggaggaatgcttacctcctaaatatattcaatctaatatttgaggacacatgggaatatatttatgattcatctgctttttaaacataagcctttgttaactgtaagttcttgaactttataaggctgctgttatttaaatgagcacagctcctgatctgcaaacagcagagcgcagggctacagcttgggggatgccagccgactcagggtggtcctrtggactgaacaatctcttgctgctgtactggagggcctgggagcttttccatcagcctcggcctgaggtgtgcactcttctcctgcccaccccaggaataaatgagattcctggttaaaaaggaccagagcagtcattttacagttgaggaaactgttgctctgagaagtgagggatttattcatgactacactgatggtgagtgcccatgtcaggtctggaaccaaagtctacccagtatccacacaccaccatccctcaggtggctctgccacagtctgatgggaggctccaaagcgggaggaagaaggaaagtcttgcccactgcatctcctcagttggccttcctctctgcctgttttccctccctacagttagcatcttaagca

SEQ ID NO: 7 - DNA, 711 bp, Homo sapiens
ttcagatttcaccaattgagaattagtaagtaatttctctgatacaggcctgaagtttaccttagtaaacactttacttccatatggtaaaaattagattttgggaggaatgcttacctcctaaatatattcaatctaatatttgaggacacatgggaatatatttatgattcatctgctttttaaacataagcctttgttaactgtaagttcttgaactttataaggctgctgttatttaaatgagcacagctcctgatctgcaaacagcagagcgcagggctacagcttgggggatgccagccgactcagggtggtcctgtggactgaacaatctcttgctgctgtactggagggcctgggagcttttccatcagcctcggcctgaggtgtgcactcttctcctgcccaccccaggaataaatgagattcctggttaaaaaggaccagagcagtcattttacagttgaggaaactgttgctctgagaagtgagggatttattcatgactacactgatggtgagtgcccatgtcaggtctggaaccaaagtctacccagtatccacacaccaccatccctcaggtggctctgccacagtctgatgggaggctccaaagcgggaggaagaaggaaagtcttgcccackgcatctcctcagttggccttcctctctgcctgttttccctccctacagttagcatcttaagca

SEQ ID NO: 8 - DNA, 706 bp, Homo sapiens
cagaataccaatgtctcctgcacttaacacattaatacaaagtttgccaattgttttgaatttccaaatgtattcctgaaaaaaaaaagaacctaaacactatattatagacatatgttagaaaagtcctagaaatgcacccaatttccttccattttactttcctacatggattgaagtcagcccctcaaaagcttttcggctgggcatggtggttcacgcccataatactagcactttgggaggccaaggtgggtggatcamctgaggtcaggaattcaagaccagcctggccaagatggtgaaaccccatctctactaaaaaatacaaaaattagcttggcgtggtagtgcgcacctgtaatcccagctactcgggaggctgaggcagacaattgcttgaacccgagagacggagggtgcagtgagccgagatcgtgctactgcactccagcctgggcaacagagcaagactccgtctcaaaaaaaaaaaaaaaaaaaaaaaagcttttcaaaagtccacccaggattttttaagacattttcccatttgtttttgcttggacgacctggttactccttgagtgcggaatatataatctaaagcatgttatgtgcaaaaggtgccatggtgttaaaaattaaaacttggaattggttgtagtaccattcgttacatttcaaagtgactaatgctgatgtcaaa

SEQ ID NO: 9 - DNA, 1532 bp, Homo sapiens
ttgttggctgtccgaggtttgctgaaacaaaggaaatgagcttggttggaaccaaagaattctgtacaaagtcaaatcctgttttgttatcacaattgacaacttaattatctctcatataaaacatgtgcatagaaccagctccctgaaagaagcaggcgtggcggcctggagctgaatcctaagacattctgtggtagtgctggcccaaggggaagacaatggagcctcagatgtcattttaagttttctagtagtcacattagaaaaagtaaaaaggaacaggtgaaattaattttaataatatattttatttaaccaaagacagttgacccctgaacaacatgggtttgaactctgtggatccacttgtatgcaaatttttttaaataaaagttacaccaagtgtgccgcctcctacttcctccatcttttctcctgccgtggcccctgctcttcctcctcctcctcctcctcagcctactcaacatgaagatgacgaggatgaacacctttatgatgatccacttccactttatcaatagtaaatatattttctcttccttataattctttctcttccttccttctttcttttcttttcttttcttttttttctttctttccctttcttttttagacagagtctcgctctgtcacccaggccggagtgcagtggcgcaatctcagttcrctgcaacctcctccacctgggttgaagtgattctcctgcctcagcctcccaagtagctgggattacaggcacccacaaccacgcctggctaatttttgtatttttagtagagatggggtttcaccatgttggccaggctggtcttgaactcctgacctcaagtgatccacccgccttagcatcccaaagttctgagattacaggcacgagccaccatgcccagcctcttttccttataattttcttaataacattttctttcctctagcttactttattgtaagaatacagtatataatacacgcaacatgaaaaatgtgtgttaattgactgtttatcttattggtaaggctgcagtcaacagtcaaccgataggtaggctattagtagttaagtttttggggagtcaagttatactcggattttcaactgcacagggagtcggtgcctctcagccatgcattgcttgagtcaactgcatatatctggaatataatcattttaatgtgtaatcaatataaaaaagttttgagatattctacacttcttaaattccagtgtgtactttatacttagagcattagccatatttcaagtggtcaacagccacatgtggctagtggctactatactgaacagggtggtagcataaactacgatattttaggttcaaaaatatgtagggctgtgagaaagcccaggaaatgtccttggtacctcgagaagccggatttaaccacggggtctctgcaatggctcaattatctcatgtgctttggttaaatgacaggaaaacaaacatcaattcacaggccaaatatgtgtgtgcacatgcaaa

SEQ ID NO: 10 - DNA, 1532 bp, Homo sapiens
ttgttggctgtccgaggtttgctgaaacaaaggaaatgagcttggttggaaccaaagaattctgtacaaagtcaaatcctgttttgttatcacaattgacaacttaattatctctcatataaaacatgtgcatagaaccagctccctgaaagaagcaggcgtggcggcctggagctgaatcctaagacattctgtggtagtgctggcccaaggggaagacaatggagcctcagatgtcattttaagttttctagtagtcacattagaaaaagtaaaaaggaacaggtgaaattaattttaataatatattttatttaaccaaagacagttgacccctgaacaacatgggtttgaactctgtggatccacttgtatgcaaatttttttaaataaaagttacaccaagtgtgccgcctcctacttcctccatcttttctcctgccgtggcccctgctcttcctcctcctcctcctcctcagcctactcaacatgaagatgacgaggatgaacacctttatgatgatccacttccactttatcaatagtaaatatattttctcttccttataattctttctcttccttccttctttcttttcttttcttttcttttttttctttctttccctttcttttttagacagagtctcgctctgtcacccaggccggagtgcagtggcgcaatctcagttcgctgcaacctcctccacctgggttgaagtgattctcctgcctcagcctcccaagtagctgggattacaggcacccacaaccacgcctggctaatttttgtatttttagtagagatggggtttcaccatgttggccaggctggtcttgaactcctgacctcaartgatccacccgccttagcatcccaaagttctgagattacaggcacgagccaccatgcccagcctcttttccttataattttcttaataacattttctttcctctagcttactttattgtaagaatacagtatataatacacgcaacatgaaaaatgtgtgttaattgactgtttatcttattggtaaggctgcagtcaacagtcaaccgataggtaggctattagtagttaagtttttggggagtcaagttatactcggattttcaactgcacagggagtcggtgcctctcagccatgcattgcttgagtcaactgcatatatctggaatataatcattttaatgtgtaatcaatataaaaaagttttgagatattctacacttcttaaattccagtgtgtactttatacttagagcattagccatatttcaagtggtcaacagccacatgtggctagtggctactatactgaacagggtggtagcataaactacgatattttaggttcaaaaatatgtagggctgtgagaaagcccaggaaatgtccttggtacctggagaagccggatttaaccacggggtctctgcaatggctcaattatctcatgtgctttggttaaatgacaggaaaacaaacatcaattcacaggccaaatatgtgtgtgcacatgcaaa

SEQ ID NO: 11 - DNA, 601 bp, Homo sapiens
caaaaccctctgatttggaattttgagttaactaaaaaattcagtcactaatttggttgcaggttgttttccagaagctttgtaaattcagctttagaattcagaacatttccatggaatgaatatcaccggtgacggtttgtgctaaggcttaagccaataacatttcccaaccaccactgaaaactgttagcaaaggtgaaaaatgcagttggagttccaagtaggggcttctgcacagcagtagtgtcctgcggctggagccaggctgcagtagtgagagcagtcgggagggaagagrggcagctgcttaagatgctaactgtagggagggaaaacaggcagagaggaaggccaactgaggagatgcagtgggcaagactttccttcttcctcccgctttggagcctcccatcagactgtggcagagccacctgagggatggtggtgtgtggatactgggtagactttggttccagacctgacatgggcactcaccatcagtgtagtcatgaataaatccctcacttctcagagcaacagtttcctcaactgtaaaatgactgctctggtcctttttaaccaggaatctcatttattc

SEQ ID NO: 12 - DNA, 957 bp, Homo sapiens
ttagacagatactacctgtactcttattctgtaatctttgttgggatggattcacatcttgcaaaggaagggaggcatgtagtataatggggcaaacagacccagctctgccactcgttagatatgtgaccttctgcaagttgcttagtgcctgtgagcttcagtgtcctcatggataagaaagatccaacaccttcttggaaggattatatcaaatgaagtaacatgagtaaagggtccagcagaatacctggcatatagtggagtcaatgaatgattaataatattattaatagtggtcatgagagaatatatgtataacatgttattatgtagactcactatatagactctattctacatagaatatagaacattatataacaaacaactataataagtagactatagtaaacaacctcactttgtctcagttgcctcatcttgatggaaaactgctctttctctcctgttaccytgacagagagcgtctacattctaaaagaaagatatttaacaaaatggttgagtacagatccaagagtcaaatagctgtctggttcaaagtccagctgtgtgattttgagctagtcacccaatctcactttgtctcagtagccttatttgtaaaaacaaggcaaattacagagccatcccctgggttgctatgaggactcaaacatgcatcccaagtgctcggtgttgctaggtatgatggctcacacctgtacattcagcactttgggaggccgaagcagaaggatcagcctgggcaacatagcaggaccccatctctacaaaacaatgtttaaaaaaaagcaaagtgctcagcacagtgactgcatcattaggattgattgtagggctcctgatgttagcacagaacaccacagccaggaagcagtctatcttgttgggtgcaaattgtaacattccatttatgtttcttccttcttt

SEQ ID NO: 13 - DNA, 512 bp, Homo sapiens
  misc_feature (256)..(256): n= absent or g or t
ttgattgatgtggataaaggagaaaactactgtttcagtgttcaagcagtgattccctcccgaacagttaaccggaagagtacagacagcccggtagagtgtatgggccaggagaaaggggaattcagaggtgagtggctctgccagccatttgcctgggggtatgggtgctgtgggtgacttctggaggagtagctccaccctcagggctgggatatacttccttggttaaatattcaggaaaacaaactgcccnngaggttttttgttgttatttgtttgttttggttttgattttgctttggtacaaaaaagattttggacatttagaaatgtttctgtgttgattgtgcccttgtattagcaggtgttttcttgagcacctgtcatgtgctaagccctctgctgagcactggatacacaaactgtgtttaggatttagcaacaagtcacagatttccctgggcattttttcatgcttaaattctaattctgggggtggcttctggacc

SEQ ID NO: 14 - DNA, 1001 bp, Homo sapiens
agacaatggagcctcagatgtcattttaagttttctagtagtcacattagaaaaagtaaaaaggaacaggtgaaattaattttaataatatattttatttaaccaaagacagttgacccctgaacaacatgggtttgaactctgtggatccacttgtatgcaaatttttttaaataaaagttacaccaagtgtgccgcctcctacttcctccatcttttctcctgccgtggcccctgctcttcctcctcctcctcctcctcagcctactcaacatgaagatgacgaggatgaacacctttatgatgatccacttccactttatcaatagtaaatatattttctcttccttataattctttctcttccttccttctttcttttcttttcttttcttttttttctttctttccctttcttttttagacagagtctcgctctgtcacccaggccggagtgcagtggcgcaatctcagttcactgcaacctcctccacctgggtygaagtgattctcctgcctcagcctcccaagtagctgggattacaggcacccacaaccacgcctggctaatttttgtatttttagtagagatggggtttcaccatgttggccaggctggtcttgaactcctgacctcaagtgatccacccgccttagcatcccaaagttctgagattacaggcacgagccaccatgcccagcctcttttccttataattttcttaataacattttctttcctctagcttactttattgtaagaatacagtatataatacacgcaacatgaaaaatgtgtgttaattgactgtttatcttattggtaaggctgcagtcaacagtcaaccgataggtaggctattagtagttaagtttttggggagtcaagttatactcggattttcaactgcacagggagtcggtgcctctcagccatgcattgcttgagtcaactgcatatatctggaatataatcattttaatgtgtaatcaatataaaa

SEQ ID NO: 15 - DNA, 511 bp, Homo sapiens
cttgatggaaaactgctctttctctcctgttaccttgacagagagcgtctacattctaaaagaaagatatttaacaaaatggttgagtacagatccaagagtcaaatagctgtctggttcaaagtccagctgtgtgattttgagctagtcacccaatctcactttgtctcagtagccttatttgtaaaaacaaggcaaattacagagccatcccctgggttgctatgaggactcaaacatgcatcccaagtgctcrgtgttgctaggtatgatggctcacacctgtacattcagcactttgggaggccgaagcagaaggatcagcctgggcaacatagcaggaccccatctctacaaaacaatgtttaaaaaaaagcaaagtgctcagcacagtgactgcatcattaggattgattgtagggctcctgatgttagcacagaacaccacagccaggaagcagtctatcttgttgggtgcaaattgtaacattccatttatgtttcttccttc

SEQ ID NO: 16 - DNA, 506 bp, Homo sapiens
  misc_feature (251)..(251): n is absent or c
gattgatgtggataaaggagaaaactactgtttcagtgttcaagcagtgattccctcccgaacagttaaccggaagagtacagacagcccggtagagtgtatgggccaggagaaaggggaattcagaggtgagtggctctgccagccatttgcctgggggtatgggtgctgtgggtgacttctggaggagtagctccaccctcagggctgggatatacttccttggttaaatattcaggaaaacaaactgncctggaggttttttgttgttatttgtttgttttggttttgattttgctttggtacaaaaaagattttggacatttagaaatgtttctgtgttgattgtgcccttgtattagcaggtgttttcttgagcacctgtcatgtgctaagccctctgctgagcactggatacacaaactgtgtttaggatttagcaacaagtcacagatttccctgggcattttttcatgcttaaattctaattctgggggtggcttctg

SEQ ID NO: 17 - DNA, 17222 bp, Homo sapiens
taccacccagtcttactttccttcattgggagtaggagaaagcctggtgatggaaggaaagcccccggggtcagaagaccagatctgcactgtgtggttaggcaagccagtttactcagatcagctggccacagggttctcatccataaaagaatgtctgtgaggttctcccatcctctgacatcctaaaatccaatgagaaagggactggtcaagccagagagattattgttatagtttagtaactttttgaacttctcagagcctccaagatagatcatggaggagggaactgttaactgctaagcttgactttactgacaggagtaaaaaaaattgtgttaaggttagggaataattttaacagtcaatttgttcttgtgaacaaatttcaacagtgaaattttagatatgtactttttaatggtgccaagcagcagttattatagatcaactgctgtttggcaccattaaaaagtacacttcgcaccgtcaaaaagtagatctggccacaattagatcagtcagggaaaaacacttcgcaatgaaatattatttaccacgttttcttcctccctcttcttgaaaatagtaatgaytttagcatttttaaatcttgaagaatgtcattccgtactgactaaaaagcctgtgcaaacacccaacatcttctctttcctgtctattttaatggatatacaaaataaatattcatctaatttatcaatatttaaggcacattgtgtatcacaatccaatggtaaacatttttcttatcaaatggctgatcttgacattgtttaaagccctatatgatttcagaaaagtcagtgactccaagtggaattgcaacaatttcttgggtcttgtaacaaacctgaagtgtaactatttctcttcagctaccttcaatcccagagagcaaagaatcacatcccaggtggagctgaagatctcttagggaaaatgactcaggctagagcctgcataaaaagaggattgcatggcttagttttgcaaacataaattttgtgggtttcagagctggcagagagctcaaagaggaaatcatcaagggcaactttctgyttcgaggaaggaaagtctacaagggctaaacctggatgtgctatttcagttcaggcaaaggaggaaaattaaaattttgcacagttttattctgttaaaacaagtggttcagtaagctaaacgagatatgtatatagtatgcattaaaatgcaagactgacttccctgggaaaacgtgggaaagaaggagagaggaaacaaacaattcttacactaagaattctccagtcgtgtcttgcaggcaatatagagtaataataataaaaatgacaggagatactttgacaggatggcttaaaatgccactcaatagatgaagagttgttctcatgcttggcaagtttacagcaaagcccagaaggagaagccagaaaataatttagaaaaaccaaagcttaaaactagtggcacacaactctggttacatttttcattttcatttttggttcctctgcatttcggtggaactcagtcccacagatgtcttcttcaatgtgggaaccatcctttctggaaccacagagctgcagatgtcacgctggaattctcccagaggcaaactgccagatgtgaggctgctcttcctcagtcactatctctggtcgtaccgggcgatgcctgagccaactgaccctcagacctgtgagccgagccggtcacaccgtggctgacaccggcattcccaccgcctttctcctgtgcgacccgctaagggccccgcgaggtgggcaggccargtattcttgaccttcgtggggtagaagaagccaccgtggctgggagagggccctgctcacagccacacgtttacttcgctgcaggtcccgagcttctgccccaggtgggcaaagcatccgggaaatgccctccgctgcccgaggggagcccagagcccgtgctttctattaaatgttgtaaatgccgcctctcccactttatcaccaaatggaagggaagaattcttccaaggcgccctccctttcctgccatagacctgcaacccacctaagctgcacgtcggagtcgcgggcctgggtgaatccgggggccttgggggacccgggcaactagacccgcctgcgtcctccagggcagctccgcgctcggtggcgcggttgaatcactggggtgagtcatcccttgcagggtcccggagtttcctaccgggaggaggcggggcaggggtgtggactcgccgggggccgcccaccgcgacggcaagtgacccgggccgggggcggggagtcgggaggagcggcgggggcgggcgccgggggcgggcagaggcgcgggagagcgcgccgccggccctttatagcgcgcggggcaccggctccccaagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggtgagtggcaccagcccctggaagcccggggcgcgccacacgcaggagggaggcgacagtcctggctggcagcgggctcgccctggttccccggggcgcccatgttgtcccccgcgcctacgggactcggctgcgctcacccagcccggcttgaatgaaccgagtccgtcgggcgccggcgggagttgcagggagggagttggcgccccagaccccgctgccccttccgctggagagttttgctcggggtgtccgagtaattggactgttgttgcataagcggacttttagctcccgctttaactctggggaaagggcttcccagtgagttgcgaccttcaatatgataggacttgtgcctgcgtctgcacgtgttggcgtgcagaggtttggatattatctttcattatatgtgcatcttcccttaataaagagcgtccctggtcttttcctggccatctttgttctaggtttgggtagaggcaatccaaaagggctggattgctgcttagattggagcaggtacaacgttgtgcatgccccgtatttctacgaggtgttcgggacggcgtagagactgggacctgctgcgtactggcaaagcagaccttcataagaaataatcctgatccaatacagccgacggtgtgacaggccacacgtccccgtgggtctctgtggaagtttcagtgtagcgacatttcagataaaagtggaaaaagtgaagtttggcttttttcatttgtatgcagtcctaactcttgtcacacgtgtgggatttatctttttccataacttactgaaaacccttcctggcgggctgaacctgactcttcctgagctgagtcctggactggcacactgatggctctgggctcttcccggtcaagttataacaaggctttgcccatgaataatttcaaacgaaaatgtcaagatccttgccggtgtcctgggattacaaggtgaatcttgtcatgaagaaattctaggtctagaaaaaatttgaagattctttttctcttgataattcactaatgaagcttttgtggttgaaaaataaaaagtgaggtttatggtgatgtcaggtgggaaggtgttttatacatcaatacattcgagtgctctgaagtgcatgtaataatagctgtttctctgttgtttaaaggcactacaaatactgtggcagcatataatttaacttggaaatcaactaatttcaagacaattttggagtgggaacccaaacccgtcaatcaagtctacactgttcaaataaggtaagctgggtacagaaaaagaaaattaaggtctttgatgtttctactgtcctatgctgaacaagaatgtctttaaagctgattactggatgaaattatttaacagatgacgaagaagaagggattcttggcaattcgctggccggtgtcatactctattaggcctgcaacatttccagaccttaaactgatagaacattttaattgttttaattgtttttggaaatgatgggagagttcctaagtggagtataaactgtggagagatgaaccatcttgagtaggcactgaagtgtgctttgggtcatgatagattaattaatctcatctaaacattgatgtctttttctgttgctgtctagactgtgaacaatgtctaacaccttagggaagaggtggggaggaatcccaatgtatacattgcccttaagcagtgtttgattcattcatctttggactccatgaatcgaaatctggtagaatacatgatcttagtggaggaggccaaatgcgtgactcactgagcctggcagagcagaaatactctgctgtctgcaccctctgggtctggtgtggctctgcttcttggtgcytcaactctgactggcagctgtccccaggaggcgataattcagcatgttcaatctaaaggttatgacttccttgatggttttcaccatattcttggcaagtttttggtttttgaaatgttctaggaggcttggtagagatcttatgaaatagagaatagctgctgtggaaattattttaatgctaattacataaaagtacaaaagtagcactagctaaaacaaaaggtattttgctgttctgttttgttttagcttgtgccaggccttttacagcattaggaatgcaacttctagataacgatgcatcttttaagtgaatgttcttgtttttcaaaatgaacttcatgacagtagttgccaaaccagcaaggagaacttgcatgcatacgtgcatgcatgtgtggatatgtatgggggtggggggagagaaagatgaaggaatttcataacatgaaataatgattacagttctggtcaaacttgtcaattcagatttcaccaattgagaattagtaagtaatttctctgatacaggcctgaagtttaccttagtaaacactttacttccatatggtaaaaattagattttgggaggaatgcttacctcctaaatatattcaatctaatatttgagggacacatgggaatatatttatgattcatctgctttttaaacataagcctttgttaactgtaagttcttgaactttataaggctgctgttatttaaatgagcacagctcctgatctgcaaacagcagagcgcagggctacagcttgggggatgccagccgactcagggtggtcctatggactgaacaatctcttgctgctgtactggagggcctgggagcttttccatcagcctcggcctgaggtgtgcactcttctcctgcccaccccaggaataaatgagattcctggttaaaaaggaccagagcagtcattttacagttgaggaaactgttgctctgagaagtgagggatttattcatgactacactgatggtgagtgcccatgtcaggtctggaaccaaagtctacccagtatccacacaccaccatccctcaggtggctctgccacagtctgatgggaggctccaaagcgggaggaagaaggaaagtcttgcccactgcatctcctcagttggccttcctctctgcctgttttccctccctacagttagcatcttaagcagctgcccctcttccctcccgactgctctcactactgcagcctggctccagccgcaggacactactgctgtgcagaagcccctacttggaactccaactgcatttttcacctttgctaacagttttcagtggtggttgggaaatgttattggcttaagccttagcacaaaccgtcaccggtgatattcattccatggaaatgttctgaattctaaagctgaatttacaaagcttctggaaaacaacctgcaaccaaattagtgactgaattttttagttaactcaaaattccaaatcagagggttttgcaatgcctggaggaaccttggaggcttttaaagtgttaatgctattaatggcattcagagggattttctacagaattgtcccttcattacctgtttatacagttttactacttaccagggtactgtataaatccttgtgctaaattttgctatagagtatgtggtccctgctgtgagctgggaggaaccaaatactgtatctctatgttacatagaaagccctaggagactttctcctgttatctgaacaactatttgctgtactgataaaaaggaaacagcatagtctcattcactttttgaaatggaaatgataaaataaaacacattttggtcattcgggaacaaaataccctctctacttttatcacataaaattaaataaatagaaaccaaaatatttcagtatcaatcttagtttgtgcactttaggataaagaatgtgtttacccaaatccttttggcctggttacttagttcagattttgaaagaaaatatatttgtggcttttatgtgtgaatttagacaatggaatccatgtggtgcctcgttttccctgagattatgtattaattcaacctgtaaatgcaaaccatctaatagtcagcgagaccctatagccctgctgcttaatgggggcacacaagggcatgcagccctcgtaccaggcagactgtgttcatattaacagcatcgtggagaaactcatgctgggggacaggggagggagatgtaaatgctcagcagggagatctggagattcctggagcaggtggagttgggacctggccttgaacgatgggtctggctctggcagtcagtaatgccaaagggaagagcagcataactgtcactttccatgggacagaagtgtgtgaatcaagttgcagtgacgcttcacctatttattattttggtcatttagaagaatttcattgtcagtagaagtcctttaaatcatttccccttcagtgacgtctcacaaaagaaagatctgtctttagctttttagtctcagactttattagacagatactacctgtactcttattctgtaatctttgttgggatggattcacatcttgcaaaggaagggaggcatgtagtataatggggcaaacagacccagctctgccactcgttagatatgtgaccttctgcaagttgcttagtgcctgtgagcttcagtgtcctcatggataagaaagatccaacaccttcttggaaggattatatcaaatgaagtaacatgagtaaagggtccagcagaatacctggcatatagtggagtcaatgaatgattaataatattattaatagtggtcatgagagaatatatgtataacatgttattatgtagactcactatatagactctattctacatagaatatagaacattatataacaaacaactataataagtagactatagtaaacaacctcactttgtctcagttgcctcatcttgatggaaaactgctctttctctcctgttaccttgacagagagcgtctacattctaaaagaaagatatttaacaaaatggttgagtacagatccaagagtcaaatagctgtctggttcaaagtccagctgtgtgattttgagctagtcacccaatctcactttgtctcagtagccttatttgtaaaaacaaggcaaattacagagccatcccctgggttgctatgaggactcaaacatgcatcccaagtgctcggtgttgctaggtatgatggctcacacctgtacattcagcactttgggaggccgaagcagaaggatcagcctgggcaacatagcaggaccccatctctacaaaacaatgtttaaaaaaaagcaaagtgctcagcacagtgactgcatcattaggattgattgtagggctcctgatgttagcacagaacaccacagccaggaagcagtctatcttgttgggtgcaaattgtaacattccatttatgtttcttccttcttttctttctttagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagagtaagtggcttgggctgtaataccgttcattcttgttagaaacgtctgaacattctcgtgatcttgtgcctttaggggctacaaaattaaaaatatttattctttttttctcagaaactggtatgtatcacagccctcttcacacattccagatgtggtaggaggttcacagaatgtgaactttggagctgatgacagtgtcatcaagtaactttctcccccagtctgtccccagaccctgttactgtcctcagtaaccggctgaatgtgtgttgggagagggcgggccagggaagcgggtagggataggaaatccaccaaggccggggttttagcttttccctatatatatatcatgtatcctgatttttctgtcccgttatcacactaaaaatcccagttgaggatttttcccaaacggtcataaatcaatgaggaaagtccatggtttccctctgagcccataattagcctaattatgctgaccttttctaatcagttggccatgatttgagttccgtgatgtgccagcacctgcccagccatctgcctgtcaccctcgttctggttttggaaaggtggaatactttcctcctcagcctttgcccctgtaagctggccctaggagccagtaaaagaatgaagagaattcctgtcaagtaggagatttattcttttgccgcaactgtggctctgagctaggcaatttagataaatgcatgtagcacattgagtagagtgaaattagcttctcttgtaaggccagctggttagaatgaaggtgttgtgtgagtgttaggcccagcgagagagaacagtttctcaaggtaggaatggtgaaaagaaggggtggacggacaaccaaccaaccatcctcctctggtatctactttgagggttgaaatagggggcctgaccccaggtgaatgtggctgccttcccagagcccccatttgcaagaccctccagacccccaggtgcttctgcttgtgtcttttgtggcaccaggcaagaatgtagcagcgtcagcagcccctctggtgactgtggcatggttgacattcatttcccccctaattaatggcatcctcatgattctcttttatattaatagttcttgagtttttttgtaagctacttcaaatcctttgttggtgcaagatagaagatattttatgtgtttgttttgcatgtgcacacacatatttggcctgtgaattgatgtttgttttcctgtcatttaaccaaagcacatgagataattgagccattgcagagaccccgtggttaaatccggcttctcgaggtaccaaggacatttcctgggctttctcacagccctacatatttttgaacctaaaatatcgtagtttatgctaccaccctgttcagtatagtagccactagccacatgtggctgttgaccacttgaaatatggctaatgctctaagtataaagtacacactggaatttaagaagtgtagaatatctcaaaacttttttatattgattacacattaaaatgattatattccagatatatgcagttgactcaagcaatgcatggctgagaggcaccgactccctgtgcagttgaaaatccgagtataacttgactccccaaaaacttaactactaatagcctacctatcggttgactgttgactgcagccttaccaataagataaacagtcaattaacacacatttttcatgttgcgtgtattatatactgtattcttacaataaagtaagctagaggaaagaaaatgttattaagaaaattataaggaaaagaggctgggcatggtggctcgtgcctgtaatctcagaactttgggatgctaaggcgggtggatcacttgaggtcaggagttcaagaccagcctggccaacatggtgaaaccccatctctactaaaaatacaaaaattagccaggcgtggttgtgggtgcctgtaatcccagctacttgggaggctgaggcaggagaatcacttcaacccaggtggaggaggttgcagtgaactgagattgcgccactgcactccggcctgggtgacagagcgagactctgtctaaaaaagaaagggaaagaaagaaaaaaaagaaaagaaaagaaaagaaagaaggaaggaagagaaagaattataaggaagagaaaatatatttactattgataaagtggaagtggatcatcataaaggtgttcatcctcgtcatcttcatgttgagtaggctgaggaggaggaggaggaggaagagcaggggccacggcaggagaaaagatggaggaagtaggaggcggcacacttggtgtaacttttatttaaaaaaatttgcatacaagtggatccacagagttcaaacccatgttgttcaggggtcaactgtctttggttaaataaaatatattattaaaattaatttcacctgttcctttttactttttctaatgtgactactagaaaacttaaaatgacatctgaggctccattgtcttccccttgggccagcactaccacagaatgtcttaggattcagctccaggccgccacgcctgcttctttcagggagctggttctatgcacatgttttatatgagagataattaagttgtcaattgtgataacaaaacaggatttgactttgtacagaattctttggttccaaccaagctcatttcctttgtttcagcaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaaggtgagcattttttaatttgtttttatgacctgttttaaattgtgaatacttgggttttacaacccatttcttccccaattcaaaaatagcagaacagagttgttgagaaggtgatggagtagaagggggagcgcgcactgtggggaggggtggacaacaggcctggtcctacctgtgactctgcactaccctgtgactctgggcagggccccctcggagacccaggttcctcagccaaccggctggatcaggtcatctctaaaggtcccgccacgctcacatttctccctctattgaggatcccaggcacaaaatttgtttttggttcaatgcataatactcccttcctttttcttttactgcagatatcttctaaaggggctcaatagggttcaatatgcctaaattggatcttctcagtcttggaaaaggcatttttagcagtgatcaagggaaactgattagcgaagtcacttctaatccttcacgtgtcagctgtgttcttgtaggctttgcttagaacctaggtttttacttccacagtgacttaataaaggggaaagaattgactcagagcccagatgaattaagaactctatctttttacagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagaggtgagtggctctgccagccatttgcctgggggtatgggtgctgtgggtgacttctggaggagtagctccaccctcagggctgggatatacttccttggttaaatattcaggaaaacaaactgccctggaggttttttgttgttatttgtttgttttggttttgattttgctttggtacaaaaaagattttggacatttagaaatgtttctgtgttgattgtgcccttgtattagcaggtgttttcttgagcacctgtcatgtgctaagccctctgctgagcactggatacacaaactgtgtttaggatttagcaacaagtcacagatttccctgggcattttttcatgcttaaattctaattctgggggtggcttctggaccagctgcagcaggacacagtagacattcgtgagtacccactgtgggctgttgccacagaggctgtagagtctaacccatcaagggaagggattgagtatatcaaatatacccacatgcatgcatgtgtgtatatggcggacacgtgtgtgtacatgcatgtgcatatgttgggagctcaggcccattgtgcgaggaacagtccctaaccggaagtgctgtgggccttcagactcttgcaggaagctgcaagcctgtgtgtctcgatccatgccttacagggaaagtattctgagtactttcagtgaagaaaagagtcaggggatataaacgatggcttacgctgggtgtggtggctcacgcctgtagtccctgcactttgggaggcccagacaggcaaatcacttgaggtcaggagtttgggaccagcctggccaacatggtaaaagcccatctctactcaaaatacaaaaagtagctgggtgtggttgcacgtgtctgtagtcccagctactcaggaggttgaggcaggagaattgcttgaacctgggaggcggaggctgaagtgagctgagattggaccactgtactccagcctgggtgacagagcgagattccatctcaaaaaaaaaaaaaaagaaacaacgaaaaaagaaatgatggcttagctccatgtgaagatgatatttgaacattttaaaacactttaaataaactgttctctcctgtttattgccactgacaggagaggtttctctttacctctggtcctgcacccctctgagccatcctacccacagccttcagtcattgtcctaaagcctagctctaattccactgcctctccttttgtgcacacacacttctctgcttccctggccgttctctatcttggagaggcatttcaaacgccacttccaccagaaggccttgctactgcaccaactagttactatctcttcttcacccaaatcctggtagcactttggatctcccactttgcacttagggttcaccttccgttataatcattgccatcaatctcagcatcgtttttaggcacttctttccagccattgttcttacctccaactacatatcttttctggactgtgcattattcagtttattaaatgcccattaaatgtgtttagccattgtcaattactctgaaacgttcaggttttgacaaattctttcctaatgtaagtgtggtggaaagagtgaaagaaagtcaaattgcacaaaaataggatggtgtaatttggggttatgccgtcaattttgtccactgataaatgggatttgagctctccaagttgactagatgccctttatttttcagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacraatggtactacaaccaattccaagttttaatttttaacaccatggcaccttttgcacataacatgctttagattatatattccgcactcaaggagtaaccaggtcgtccaagcaaaaacaaatgggaaaatgtcttaaaaaatcctgggtggacttttgaaaagctttttttttttttttttttttgagacggagtcttgctctgttgcccaggctggagtgcagtagcacgatctcggctcactgcaccctccgtctctcgggttcaagcaattgtctgcctcagcctcccgagtagctgggattacaggtgcgcactaccacaccaagctaatttttgtattttttagtagagatggggtttcaccatcttggccaggctggtcttgaattcctgacctcaggtgatccacccaccttggcctcccaaagtgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattggcaaactttgtattaatgtgttaagtgcaggagacattggtattctgggcaccttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggttttctatatggggattttctatttatgtaggtaatattgttctatttgtatatattgagataatttatttaatatactttaaataaaggtgactgggaattgttactgttgtacttattctatcttccatttattatttatgtacaatttggtgtttgtattagctctactacagtaaatgactgtaaaattgtcagtggcttacaacaacgtatctttttcgcttataatacattttggtgactgtaggctgactgcacttcttctcaatgttttctcattctaggatgcaaaccaatggagaagcccctaattagatcagggcagagggaaaaacaaaaaactggtagaaaccggcaaccacagcttcaagctttaagcccatctcctacacttctgctctgtacgtgcccattgtcacttctgttcacatgctactgtcccaagcaagtgaccaagcctgacaatactttgtctactggagtcactgcaaggcacatgacggggcagggatgtcgtcttacagggaagagaaaagataatgctctctactgcagacttggagagatttcttcccattggcagtagtttgactaattggagatgagaaaaaaagaaacattcttgggatgattgtattgaaacaaaattaggtaaaaggacaatataggatagggagagatataagtggaatgagatctctagagtccattaaaagcaagctagattgagagctcttggagggcagggactgggcctagctcatggtttacagctcttgagggtgactgcacagtggactcagtcagtcatggctgagttgagttggctttattatctctagaatgtagttctccattcaaatgcaaaacagccttattctctcaactgtagtaaaagaggattctaactagagatgaaaaaggttcttggcacctactgggccacaagactgcttttgcttcccactgtggaagggggcactgttatctaggaaaaacatccagatggcacacctactcagaagtggttccacaaagaaattgagcaagcattcagatgagcagcctcagctcactatctagctgttaataaattttaagtccgttttgttcctttgcaccaaagaaattgaacaatgtcaataaaattccaaacgtgaactgttttatgtaacaccacatcctaaggaagaatgcctgaaacaaacctttgatgtcctcataaaaatgatagccattttggagaaattatggaaaacagaccagtggttggtttatttggttggttttgtttattattacttttttttggccatcaacacagaggctagagaggttgaatcttgcactgttgtagtgttttgttatacagagaaaagggatgtatttgcagcagagtcaacaaaaaagaaactggcatgggggcacatcctcaaggtgcttcaacctttatctcaaattcctgctcacatggggctacctgggacaaagagcttagtgcattacatgaggaggcagaacaagctacagcagaaggtgcctaaccagatatttccctgccaggattaattatagcattatgtaatttcttggcctcattatctaaggaatttgaattattccactaaggaaaatactggcagatagaaaacaggtctgggtactaatgaggaatctggctgatcacaaggaagggattcattatggactggaatgaggctgaactgcaataatcagaaagcttccacacagcaaatgttaaccactccctactttgccacatccctctgaattgctgagacctaacagccgatactaaaaatcagcactgtgattgacactggggcagaaaccataccaaagactgttggctgcaatccagaaactgttgccctccagtggaccaaattatacttcattacttgaccagtaatgtaccccatagagctaattgttagttaaccaaactatcatgcattctttgttttgttttgtgagacagagtctggctttgtctccaggctggagtgcagtggtgagatctcggctcactgcaacctctgcctcccaggttcaagcgattctcgtgcctcagccttctgagtagctgggattacaggtgtgagccaccatgcctggctaatttttgtatttttagtagagatagggttttgccatgttggcaaactcctgacttcaagtgatccacccacctcggcctcccaaagtgctgagattataggcgtgagccaccatgcccagccctttcatgcgttctaaagatatttttccaatcttaaattattaactgaatttggcttacatataagaaactaatactcatgaaatccaaagacattttagcttaatttcagctgatggcttataatctaaggaactgccccttaaacaattatctctattcatcaaatggtgaataaactcgttcccaaatg

(19) United States
(12) Patent Application Publication
(10) Pub. No.: US 2009/0148458 A1
(43) Pub. Date: Jun. 11, 2009

(54) COAGULATION FACTOR III POLYMORPHISMS ASSOCIATED WITH PREDICTION OF SUBJECT OUTCOME AND RESPONSE TO THERAPY

(75) Inventors: James Russell, Vancouver (CA); Keith R. Walley, North Vancouver (CA)

Correspondence Address: BROWDY AND NEIMARK, P.L.L.C.;624 NINTH STREET, NW, SUITE 300, WASHINGTON, DC, 20001-5303, US

(73) Assignee: THE UNIVERSITY of BRITISH COLUMBIA, Vancouver (CA)

(21) Appl. No.: 12/090,613

(22) PCT Filed: Jun. 23, 2006
(86) PCT No.: PCT/CA2006/001058

§ 371 Date: Oct. 8, 2008

Related U.S. Application Data
(60) Provisional application No. 60/693,042, filed on Jun. 23, 2005.
(60) Provisional application No. 60/693,043, filed on Jun. 23, 2005.

(51) Int. Cl. A61K 39/395; C12Q 1/68; C07H 21/04; C07K 2/00; A61K 38/16

(52) U.S. Cl. 424/141.1; 435/6; 536/24.3; 530/322; 514/12

(57) ABSTRACT

The invention provides methods and kits for obtaining a prognosis for a subject having or at risk of developing an inflammatory condition or hypertension. The method generally comprises determining a coagulation factor III genotype(s) of a subject for one or more SNPs, comparing the determined genotype with known genotypes for the polymorphism that correspond with the ability of the subject to recover from the inflammatory condition and identifying subjects based on their prognosis. The invention also provides for methods of identifying potential subjects having an inflammatory condition who are more likely to benefit from treatment with an anti-inflammatory agent or anti-coagulant agent and subsequent to treatment recover from the inflammatory condition. The invention also provides for methods of treating such subjects with an anti-inflammatory agent or anti-coagulant agent based on the subject's genotype.

### FIELD OF THE INVENTION

The field of the invention relates to the assessment of subjects with an inflammatory condition and/or treatment of subjects with an inflammatory condition.

### BACKGROUND OF THE INVENTION

Genotype has been shown to play a role in the prediction of subject outcome in inflammatory and infectious diseases (MCGUIRE W. et al. Nature (1994) 371:508-10; NADEL S. et al. Journal of Infectious Diseases (1996) 174:878-80; MIRA J P. et al. JAMA (1999) 282:561-8; MAJETSCHAK M. et al. Ann Surg (1999) 230:207-14; STUBER F. et al. Crit. Care Med (1996) 24:381-4; STUBER F. et al. Journal of Inflammation (1996) 46:42-50; and WEITKAMP J H. et al. Infection (2000) 28:92-6). Furthermore, septic and non-septic stimuli such as bacterial endotoxin and cardiopulmonary bypass (CPB), respectively, activate the coagulation system and trigger a systemic inflammatory response syndrome (SIRS).

Genotype can alter response to therapeutic interventions. Genentech's HERCEPTIN® was not effective in its overall Phase III trial but was shown to be effective in a genetic subset of patients with human epidermal growth factor receptor 2 (HER2)-positive metastatic breast cancer. Similarly, Novartis' GLEEVEC® is only indicated for the subset of chronic myeloid leukemia patients who carry a reciprocal translocation between chromosomes 9 and 22.

Coagulation Factor III (F3) also known as Tissue Factor (TF) or thromboplastin is a 47 kDa trans-membrane glycoprotein found in numerous tissues and is involved in the activation of a coagulation response. Binding of F3 to activated factor VII activates coagulation factor X (FX) to FXa to initiate the extrinsic coagulation cascade. The F3 sequence maps to chromosome 1p22-p21 and extends over 17 kb. Representative Homo sapiens F3 gene sequences are listed in GenBank under accession numbers AF540377.1 (GI:22536175) (17222 bp) and J02846.1 (GI:339505). The human F3 gene has 6 exons. Tissue Factor (or Coagulation Factor III) has been studied on blood monocytes in early infants in association with infection (RIVERS RPA. et al. Pediatric Research (1992) 31(6):567-573) and in baboons in association with lethal E. coli sepsis (DRAKE T A. et al. Am. J. of Pathology (1993) 142(5):1458-1470).

Expression of F3 is upregulated in various cardiovascular phenotypes including primary pulmonary hypertension (COLLADOS M T et al. Heart Vessels (2003) 18:12-7) and systemic hypertension (FELMEDEN D C et al. Am J Cardiol (2003) 92(4):400-5). Furthermore, systemic hypertension is viewed as a risk factor for vascular thrombosis (SARDO M A et al., J Hypertens (2006) 24(4):731-6). F3 levels are higher in hypertensive individuals with atherosclerosis (i.e., complicated hypertension) than those with uncomplicated hypertension (WELTY-WOLF K E et al. Semin Hematol (2001) 38(4 Suppl 12):35-8) suggesting that F3 may play a role in the formation of atherosclerotic lesions by promoting the mobilization and accumulation of vascular smooth muscle cells or through the generation of thrombi (STEFFEL J et al. Circulation (2006) 113(5):722-31). Hypertension-associated increases in F3 expression are also observed in patients with diabetes mellitus (LIM H S et al., Diabetic Medicine (2005) 22(3):249-255). Furthermore, hyperglycemia increases fibrin deposition in renal tubular cells because of increased F3 expression, suggesting that F3 plays a role in diabetic nephropathy (SOMMELIJER D W et al., Neph Exp Nephrol (2005) 101(3):886-94). Smoking is another cardiovascular risk factor that induces F3 expression in endothelial cells through the actions of nicotine and may be the principal mechanism for increased risk of stroke and myocardial infarction (CIRILLO P et al., J Thromb Haemost (2006) 4:453-8). Another association between increased F3 expression and hypertension occurs in preeclampsia (i.e. pregnancy-associated hypertension) where both monocytes and placental tissue synthesize increased levels of F3 (DECHEND R et al., J Soc Gynecol Investig 13(2):79-86).

A number of polymorphisms have been observed in the scientific literature and investigated for associations with various disease indications. Several polymorphisms in the promoter region of the F3 gene (a C/T transition at position −1812, a C/T transition at position −1322, a 18-base insertion/deletion (indel) at position −1208, and an A/G transition at position-603) were investigated for association with venous thromboembolism and myocardial infarction (MI) (ARNAUD E. et al. Arterioscler Thromb Vasc Biol (2000) 20:892-898). The positions of polymorphisms −1812, −1322 and −603 correspond to the polymorphisms described herein as 599 (rs958587), 1089 (rs3761955) and 1826 (rs1361600) of SEQ ID NO:3, 5, 4 respectively. The −1208 deletion has been observed to be associated with reduced tissue factor expression and a decreased risk of developing venous thrombosis. In contrast, the −1208 deletion has been associated with increased F3 mRNA and F3 expression in human umbilical vein endothelial cell (HUVEC) culture (TERRY C M. et al. J. Thrombosis and Haemostasis (2004) 2:1351-1358). A relationship between the number of −1208 insertion alleles, resulting in a cumulative increase in F3 expression and age at first coronary bypass operation has also been suggested (DONAHUE B S. et al. Anesthesiology (2003) 99:1287-1294). Thus, it is unclear what role the −1208 polymorphism plays in tissue factor expression in cardiovascular events. The −603 G allele has also been associated with miocardial infarction (MI) (OTT I. et al. Atherosclerosis (2004) 177:189-191). The −603 G allele has also been associated with increased monocyte F3 mRNA expression (RENY J-L. et al. Thromb Haemost (2004) 91:248-254).

### SUMMARY OF THE INVENTION

This invention is based in part on the surprising discovery that particular single nucleotide polymorphisms (SNPs) from the human coagulation factor III (F3) sequence can be predictors of subject outcome from an inflammatory condition.

Furthermore, various F3 SNPs are provided which are useful for subject screening, as an indication of subject outcome, or for prognosis for recovery from an inflammatory condition.

This invention is also based in part on the identification the particular nucleotide at the site of a given SNP which is associated with a decreased likelihood of recovery from an inflammatory condition (i.e. ‘risk genotype’ or ‘risk allele’) or an increased likelihood of recovery from an inflammatory condition (i.e. ‘protective genotype’ or ‘protective allele’). Furthermore, this invention is in part based on the discovery that the risk genotype or allele may be predictive of increased responsiveness to the treatment of the inflammatory condition with the anti-inflammatory agent or the anti-coagulant agent. The anti-inflammatory agent or the anti-coagulant agent may be activated protein C. The inflammatory condition may be SIRS, sepsis or septic shock.

This invention is also based in part on the identification the particular nucleotide at the site of a given SNP which is associated with an increased likelihood of hypertension and which may also be predictive of the severity of numerous cardiovascular phenotypes involving hypertension, such as systemic hypertension, pulmonary hypertension, atherosclerosis, diabetes mellitus, preeclampsia, and hypertension associated with smoking.

Previous studies have not examined the association of F3 polymorphisms with clinical outcome in critical illness and cardiovascular phenotypes involving hypertension such as systemic inflammatory response syndrome (SIRS), sepsis, septic shock, systemic hypertension, pulmonary hypertension, atherosclerosis, diabetes mellitus, preeclampsia, and smoking. Similarly, these polymorphisms have not been associated with improved responses to therapy.

In accordance with one aspect of the invention, methods are provided for obtaining a prognosis for a subject having, or at risk of developing, an inflammatory condition, the method including determining a genotype of said subject which includes one or more polymorphic sites in the subject's coagulation factor III (F3) sequence, wherein said genotype is indicative of an ability of the subject to recover from the inflammatory condition. The polymorphic site may be selected from one or more of the following: rs958587; rs3761955; rs1361600; rs696619; and rs3354; or one or more polymorphic sites in linkage disequilibrium (LD) thereto. The polymorphic sites in linkage disequilibrium thereto may be selected from one or more of the polymorphic sites listed in TABLE 1B. The polymorphic sites listed in TABLE 1B that are in LD may be selected from one or more of the following: rs958587; rs3761955; rs1361600; rs696619; rs762485; rs841697; rs1144300; rs3917615; rs2794470; rs841695; rs762484; rs841696; rs3917628; rs2391424; and rs841691. Alternatively, the polymorphic sites in LD with one or more of: rs958587; rs3761955; rs1361600; rs696619; and rs3354 may be determined by identifying SNPs that have a r² value≧0.8. Alternatively, the polymorphic sites in LD may be determined by identifying SNPs that have a r² value≧0.5. Also, the polymorphic sites in LD may be determined by identifying SNPs that have a r² value≧0.6. The polymorphic sites in LD may be determined by identifying SNPs that have a r value ≧0.7. The polymorphic sites in LD may be determined by identifying SNPs that have a r² value≧0.85. The polymorphic sites in LD may be determined by identifying SNPs that have a r² value≧0.75. The polymorphic sites in LD may be determined by identifying SNPs that have a r² value≧0.9. The polymorphic sites in LD may be determined by identifying SNPs that have a r² value≧0.95. Alternatively, LD may be determined using a D′ value. Particularly, a D′ of: ≧0.5; ≧0.6; ≧0.7; ≧0.75; ≧0.8; ≧0.85; ≧0.9; or ≧0.95.

The method may further include comparing the genotype so determined with known genotypes which are known to be indicative of a prognosis for recovery from: (i) the subject's type of inflammatory condition; or (ii) another inflammatory condition. The method may further include determining the coagulation factor III sequence information for the subject. Determining of genotype may be performed on a nucleic acid sample from the subject. The method may further include obtaining a nucleic acid sample from the subject.

Determining of genotype may include one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data.

The risk allele of the subject may be indicative of a decreased likelihood of recovery from an inflammatory condition or an increased risk of having a poor outcome. The risk allele may be indicative of a prognosis of severe cardiovascular, respiratory, neurological, coagulation, hepatic or renal dysfunction. The risk allele may be selected from one or more of the following: rs958587C; rs3761955G; rs1361600A; rs696619C; and rs3354T; or one or more polymorphic sites in linkage disequilibrium thereto as listed in TABLE 1B.

The protective allele of the subject may be indicative of an increased likelihood of recovery from an inflammatory condition. The protective allele may be indicative of a prognosis of less severe cardiovascular, respiratory, neurological, coagulation, hepatic or renal dysfunction. The protective allele may be selected from one or more of the following: rs958587T; rs3761955A; rs1361600G; rs696619T; and rs3354C; or one or more polymorphic sites in linkage disequilibrium thereto as listed in TABLE 1B.

The inflammatory condition may be selected from the group including: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. The inflammatory condition may be SIRS. The inflammatory condition may be sepsis. The inflammatory condition may be septic shock.

The 4524 SNP (rs696619) may be indicative of subject prognosis for a Caucasian population. The 599 SNP (rs958587) may be indicative of subject prognosis for an Asian population. The 1089 SNP (rs3761955) may be indicative of subject prognosis for an Asian population. The 1826 SNP (rs1361600) may be indicative of subject prognosis for an Asian population. The 13925 SNP (rs3354) may be indicative of subject prognosis for a Caucasian population.

In accordance with another aspect of the invention, methods are provided for identifying a polymorphism in a F3 sequence that correlates with prognosis of recovery from an inflammatory condition in a subject, the method including: (a) obtaining an F3 sequence information from a group of subjects with an inflammatory condition; (b) identifying at least one polymorphic nucleotide position in the F3 sequence in the subjects; (c) determining a genotype at the polymorphic site for individual subjects in the group; (d) determining recovery capabilities of individual subjects in the group from the inflammatory condition; and (e) correlating genotypes determined in step (c) with the recovery capabilities determined in step (d) thereby identifying said F3 polymorphisms that correlate with recovery. Obtaining F3 sequence information from a group of subjects may include obtaining nucleic acid samples from the subjects.

In accordance with another aspect of the invention, a kit for determining a genotype at a defined nucleotide position within a polymorphic site in a F3 sequence is provided, wherein knowledge of the genotype provides a prognosis of the subject's ability to recover from an inflammatory condition, the kit including: a restriction enzyme capable of distinguishing alternate nucleotides at the polymorphic site; or a labeled oligonucleotides or peptide nucleic acid that is sufficiently complementary to an alternate nucleotide sequence at the polymorphic site so as to be capable of specifically hybridizing to said alternate nucleotide sequence, whereby the genotype of the polymorphic site may be determined. Optionally, instructions for use in determining the genotype may be included.

The polymorphic site may be selected from one or more of the following: rs958587; rs3761955; rs1361600; rs696619; and rs3354; or one or more polymorphic sites in linkage disequilibrium thereto. The kit may further include an oligonucleotides or peptide nucleic acid or a set of oligonucleotides or peptide nucleic acids suitable to amplify a region including the polymorphic site. The may further include a polymerizing agent.

In accordance with another aspect of the invention, methods are provided for selecting a group of subjects for determining the efficacy of a candidate drug known or suspected of being useful for the treatment of an inflammatory condition, the method including determining a genotype for one or more polymorphic sites in a F3 sequence for each subject, wherein said genotype is indicative of the subject's ability to recover from the inflammatory condition and sorting subjects based on their genotype. The method may further include, administering the candidate drug to the subjects or a subset of subjects and determining each subject's ability to recover from the inflammatory condition. The method may further include, comparing subject response to the candidate drug based on genotype of the subject.

In accordance with another aspect of the invention, oligonucleotides or peptide nucleic acids of about 10 to about 400 nucleotides that hybridize specifically to a sequence contained in a human target sequence including of any one or more of SEQ ID NO:1-17, a complementary sequence of the target sequence or RNA equivalent of the target sequence and wherein the oligonucleotides or peptide nucleic acid is operable in determining a polymorphism genotype are provided.

In accordance with another aspect of the invention, oligonucleotides or peptide nucleic acids of about 10 to about 400 nucleotides that hybridize specifically to a sequence contained in a human target sequence including of one or more of SEQ ID NO: 1-17, a complementary sequence of the target sequence or RNA equivalent of the target sequence and wherein said hybridization is operable in determining a polymorphism genotype are provided.

In accordance with another aspect of the invention, there are provided oligonucleotides or peptide nucleic acid probes selected from the group including of: (a) a probe that hybridizes under high stringency conditions to a nucleic acid molecule including SEQ ID NO:17 having a C at position 599 but not to a nucleic acid molecule including SEQ ID NO:17 having a T at position 599; (b) a probe that hybridizes under high stringency conditions to a nucleic acid molecule including SEQ ID NO:17 having a T at position 599 but not to a nucleic acid molecule including SEQ ID NO:17 having a C at position 599; (c) a probe that hybridizes under high stringency conditions to a nucleic acid molecule including SEQ ID NO:17 having a C at position 1089 but not to a nucleic acid molecule including SEQ ID NO:17 having a T at position 1089; (d) a probe that hybridizes under high stringency conditions to a nucleic acid molecule including SEQ ID NO:17 having a T at position 1089 but not to a nucleic acid molecule including SEQ ID NO:17 having a C at position 1089; (e) a probe that hybridizes under high stringency conditions to a nucleic acid molecule including SEQ ID NO:17 having a G at position 1826 but not to a nucleic acid molecule including SEQ ID NO:17 having a A at position 1826; (f) a probe that hybridizes under high stringency conditions to a nucleic acid molecule including SEQ ID NO:17 having a A at position 1826 but not to a nucleic acid molecule including SEQ ID NO:17 having a G at position 1826; (g) a probe that hybridizes under high stringency conditions to a nucleic acid molecule including SEQ ID NO:17 having a C at position 4524 but not to a nucleic acid molecule including SEQ ID NO:17 having a T at position 4524; (h) a probe that hybridizes under high stringency conditions to a nucleic acid molecule including SEQ ID NO:17 having a T at position 4524 but not to a nucleic acid molecule including SEQ ID NO:17 having a C at position 4524; (i) a probe that hybridizes under high stringency conditions to a nucleic acid molecule including SEQ ID NO:17 having a G at position 13925 but not to a nucleic acid molecule including SEQ ID NO:17 having an A at position 13925; and (j) a probe that hybridizes under high stringency conditions to a nucleic acid molecule including SEQ ID NO: 17 having an A at position 13925 but not to a nucleic acid molecule including SEQ ID NO: 17 having a G at position 13925.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 599 is C, under conditions in which the oligonucleotides or peptide nucleic acid will not substantially hybridize to a nucleic acid molecule including of SEQ ID NO:17 wherein the nucleotide at position 599 is T.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 599 is T, under conditions in which the oligonucleotides or peptide nucleic acid will not substantially hybridize to a nucleic acid molecule including of SEQ ID NO:17 wherein the nucleotide at position 599 is C.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 1089 is C, under conditions in which the oligonucleotides or peptide nucleic acid will not substantially hybridize to a nucleic acid molecule including of SEQ ID NO:17 wherein the nucleotide at position 1089 is T.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 1089 is T, under conditions in which the oligonucleotides or peptide nucleic acid will not substantially hybridize to a nucleic acid molecule including of SEQ ID NO:17 wherein the nucleotide at position 1089 is C.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 1826 is A, under conditions in which the oligonucleotides or peptide nucleic acid will not substantially hybridize to a nucleic acid molecule including of SEQ ID NO:17 wherein the nucleotide at position 1826 is G.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 1826 is G, under conditions in which the oligonucleotides or peptide nucleic acid will not substantially hybridize to a nucleic acid molecule including of SEQ ID NO:17 wherein the nucleotide at position 1826 is A.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 4524 is C, under conditions in which the oligonucleotides or peptide nucleic acid will not substantially hybridize to a nucleic acid molecule including of SEQ ID NO: 17 wherein the nucleotide at position 4524 is T.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 4524 is T, under conditions in which the oligonucleotides or peptide nucleic acid will not substantially hybridize to a nucleic acid molecule including of SEQ ID NO:17 wherein the nucleotide at position 4524 is C.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 13925 is G, under conditions in which the oligonucleotides or peptide nucleic acid will not substantially hybridize to a nucleic acid molecule including of SEQ ID NO:17 wherein the nucleotide at position 13925 is A.

In accordance with another aspect of the invention, there is provided an array of nucleic acid molecules attached to a solid support, the array including an oligonucleotide or peptide nucleic acid that will hybridize to a nucleic acid molecule including of SEQ ID NO:17, wherein the nucleotide at position 13925 is A, under conditions in which the oligonucleotides or peptide nucleic acid will not substantially hybridize to a nucleic acid molecule including of SEQ ID NO:17 wherein the nucleotide at position 13925 is G.

There may be two or more oligonucleotides or peptide nucleic acid molecules as described herein. There may also be three or more oligonucleotides or peptide nucleic acids or nucleic acid molecules. Alternatively, there may be four or more oligonucleotides or peptide nucleic acids or nucleic acid molecules. There may be five or more oligonucleotides or peptide nucleic acids or nucleic acid molecules. There may be six or more oligonucleotides or peptide nucleic acids or nucleic acid molecules. There may be seven or more oligonucleotides or peptide nucleic acids or nucleic acid molecules. There may be eight or more oligonucleotides or peptide nucleic acids or nucleic acid molecules. There may be nine or more oligonucleotides or peptide nucleic acids or nucleic acid molecules. There may be ten or more oligonucleotides or peptide nucleic acids or nucleic acid molecules. There may be eleven or more oligonucleotides or peptide nucleic acids or nucleic acid molecules.

The oligonucleotides or peptide nucleic acids may alternatively be of about 10 to about 400 nucleotides, about 15 to about 300 nucleotides. The oligonucleotides or peptide nucleic acids may alternatively be of about 20 to about 200 nucleotides, about 25 to about 100 nucleotides. The oligonucleotides or peptide nucleic acids may alternatively be of about 20 to about 80 nucleotides, about 25 to about 50 nucleotides.

The oligonucleotides or peptide nucleic acids may further include one or more of the following: a detectable label; a quencher; a mobility modifier; a contiguous non-target sequence situated 5′ or 3′ to the target sequence.

In accordance with another aspect of the invention, there is provided a method of treating an inflammatory condition in a subject in need thereof, the method including administering to the subject an anti-inflammatory agent or an anti-coagulant agent, wherein said subject has a F3 sequence risk genotype.

In accordance with another aspect of the invention, there is provided a method of treating an inflammatory condition in a subject in need thereof, the method including: selecting a subject having a risk genotype in their F3 sequence; and administering to said subject an anti-inflammatory agent or an anti-coagulant agent.

In accordance with another aspect of the invention, there is provided a method of treating a subject with an inflammatory condition by administering an anti-inflammatory agent or an anti-coagulant agent, the method including administering the anti-inflammatory agent or the anti-coagulant agent to subjects that have a risk genotype in their F3 sequence, wherein the risk genotype is predictive of increased responsiveness to the treatment of the inflammatory condition with the anti-inflammatory agent or the anti-coagulant agent.

In accordance with another aspect of the invention, there is provided a method of identifying a subject with increased responsiveness to treatment of an inflammatory condition with an anti-inflammatory agent or an anti-coagulant agent, including the step of screening a population of subjects to identify those subjects that have a risk genotype in their F3 sequence, wherein the identification of a subject with a risk genotype in their F3 sequence is predictive of increased responsiveness to the treatment of the inflammatory condition with the anti-inflammatory agent or the anti-coagulant agent.

In accordance with another aspect of the invention, there is provided a method of selecting a subject for the treatment of an inflammatory condition with an anti-inflammatory agent or an anti-coagulant agent, including the step of identifying a subject having a risk genotype in their F3 sequence, wherein the identification of a subject with the risk genotype is predictive of increased responsiveness to the treatment of the inflammatory condition with the anti-inflammatory agent or the anti-coagulant agent.

In accordance with another aspect of the invention, there is provided a method of treating an inflammatory condition in a subject, the method including administering an anti-inflammatory agent or an anti-coagulant agent to the subject, wherein said subject has a risk genotype in their F3 sequence.

In accordance with another aspect of the invention, there is provided a method of treating an inflammatory condition in a subject, the method including: identifying a subject having a risk genotype in their F3 sequence; and administering an anti-inflammatory agent or an anti-coagulant agent to the subject.

In accordance with another aspect of the invention, there is provided a use of an anti-inflammatory agent or an anti-coagulant in the manufacture of a medicament for the treatment of an inflammatory condition, wherein the subjects treated have a risk genotype in their F3 sequence.

In accordance with another aspect of the invention, there is provided a use of an anti-inflammatory agent or an anti-coagulant in the manufacture of a medicament for the treatment of an inflammatory condition in a subset of subjects, wherein the subset of subjects have a risk genotype in their F3 sequence.

The method or use may further include determining the subject's APACHE II score as an assessment of subject risk. The method or use may further include determining the number of organ system failures for the subject as an assessment of subject risk. The subject's APACHE II score may be indicative of an increased risk when ≧25. 2 or more organ system failures may be indicative of increased subject risk.

The risk allele may be selected from one or more of the following: rs3761955G; and rs1361600A; or a polymorphic site in linkage disequilibrium thereto as set out in TABLE 1B. The genotype of the subject may be indicative of an increased risk of poor outcome from an inflammatory condition. A subject having an increased risk of poor outcome from an inflammatory condition may be preferentially selected for administration the anti-inflammatory agent or the anti-coagulant agent. The anti-inflammatory agent or the anti-coagulant agent may be selected from any one or more of the following: activated protein C; tissue factor pathway inhibitors; platelet activating factor hydrolase; PAF-AH enzyme analogues; antibody to tumor necrosis factor alpha; soluble tumor necrosis factor receptor-immunoglobulin G1; procysteine; elastase inhibitor; human recombinant interleukin 1 receptor antagonists; and antibodies, inhibitors and antagonists to endotoxin, tumour necrosis factor receptor, interleukin-6, high mobility group box, tissue plasminogen activator, bradykinin, CD-14, F3, Factor VII, Factor X and interleukin-10. The anti-inflammatory agent or the anti-coagulant agent may be activated protein C. The anti-coagulant agent may be drotecogin alfa activated. The anti-inflammatory agent or the anti-coagulant agent may be a monoclonal antibody to F3.

In accordance with another aspect of the invention, there is provided a method for obtaining a prognosis for a subject having, or at risk of developing, hypertension, the method may include determining a genotype of said subject which includes one or more polymorphic sites in the subject's coagulation factor III (F3) sequence, wherein said genotype is indicative of the subject's likelihood of developing hypertension. The polymorphic site indicative of hypertension may be rs3354; or one or more polymorphic sites in linkage disequilibrium thereto. The one or more polymorphic sites in linkage disequilibrium thereto may be selected from one or more of the following polymorphic sites: rs841696; rs3917628; rs3917629; and rs841691. The polymorphic site in linkage disequilibrium with rs3354 may have a r² value ≧0.8. The one or more polymorphic sites in linkage disequilibrium thereto may be selected from the following: rs841696; rs3917628; rs3917629T; and rs841691. The method may further include determining the coagulation factor III sequence information for the subject. The determining of genotype may be performed on a nucleic acid sample from the subject. The method may include obtaining a nucleic acid sample from the subject.

The risk allele of the subject may be indicative of an increased likelihood of hypertension. The risk allele may be rs3354T; or one or more polymorphic sites in linkage disequilibrium selected from: rs841696A; rs3917628C; rs3917629TG; and rs841691A. The protective allele of the subject may be indicative of a decreased likelihood of hypertension. The protective allele may be rs3354C; or one or more polymorphic sites in linkage disequilibrium selected from: rs841696G; rs3917628-; rs3917629-; and rs841691C. Furthermore, there are numerous cardiovascular phenotypes involving hypertension such as systemic hypertension, pulmonary hypertension, atherosclerosis, diabetes mellitus, preeclampsia, and hypertension associated with smoking, the severity of which may be predicted based on F3 alleles.

The above identified sequence positions refer to one strand of the F3 sequence as indicated. It will be apparent to a person skilled in the art that analysis could be conducted on the complimentary strand to determine the allele at a given position.

### BRIEF DESCRIPTION OF THE DRAWINGS

FIG. 1 Shows Kaplan-Meier survival curves for patients with the coagulation factor III (F3) 1826 AA or AG genotype (AA/AG) and who were or who were not treated with XIGRIS™ (dashed line=XIGRIS™ treated, solid line=matched controls (not XIGRIS™ treated).

FIG. 2 Shows Kaplan-Meier survival curves for patients with the F3 1826 GG genotype and who were or who were not treated with XIGRIS™ (dashed line=XIGRIS™ treated, solid line=matched controls (not XIGRIS™ treated).

FIG. 3 Shows Kaplan-Meier survival curves for patients with the F3 1089 G allele and who were or who were not treated with XIGRIS™ (dashed line ═XIGRIS™ treated, solid line=matched controls (not XIGRIS™ treated).

FIG. 4 Shows Kaplan-Meier survival curves for patients with the F3 1089 A allele who were or who were not treated with XIGRIS™ (dashed line ═XIGRIS™ treated, solid line=matched controls (not XIGRIS™ treated).

### DETAILED DESCRIPTION OF THE INVENTION

### 1. Definitions

In the description that follows, a number of terms are used extensively, the following definitions are provided to facilitate understanding of the invention.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). “Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T, while a nucleotide represented by the symbol B may be either G or C or T, and a nucleotide represented by the symbol H may be either A or C or T. Furthermore, a deletion or an insertion may be represented by either a “−” or “del” and “+” or “ins” or “I” respectively. Alternatively, polymorphisms may be represented as follows −/C (SEQ ID NO:16), wherein the allele options at a polymorphic site are separated by a forward slash (“/”). For example, “−/C” may be either a deletion or C.

A “polymorphic site” or “polymorphism site” or “polymorphism” or “single nucleotide polymorphism site” (SNP site) as used herein is the locus or position within a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, enhancers and introns) of genes.

A “risk genotype” as used herein refers to an allelic variant (genotype) at one or more polymorphic sites within the F3 sequence described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Such “risk alleles” or “risk genotype” may be selected from positions 599C, 1089G, 1826A, 4524C or 13925T of SEQ ID NO: 1-5 (F3) or rs958587C; rs3761955G; rs1361600A; rs696619C; and rs3354T.

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High stringency hybridization is relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

As used herein “haplotype” is a set of alleles situated close together on the same chromosome that tend to be inherited together. Such allele sets occur in patterns which are called haplotypes. Haplotype is commonly used in reference to the linked genes of the major histocompatibility complex. A “clade” is a group of haplotypes that are closely related phylogenetically. For example, if haplotypes are displayed on a phylogenetic (evolutionary) tree a clade includes all haplotypes contained within the same branch.

Accordingly, a specific SNP allele at one SNP site is often associated with a specific SNP allele at a nearby second SNP site. When this occurs, the two SNPs are said to be in linkage disequilibrium (LD) because the two SNPs are not just randomly associated (i.e., in linkage equilibrium).

Furthermore, the preferential occurrence of a disease gene in association with specific alleles or haplotypes, such as SNPs, is also described as being in LD. This sort of disequilibrium generally implies that most of the disease chromosomes carry the same mutation and the markers being tested are relatively close to the disease gene(s).

In SNP-based association analysis and linkage disequilibrium mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs are tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For a LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs).

In population genetics, LD refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as 599 of SEQ ID NO: 1 (see Akey, J. et al. (2001). Haplotypes vs single marker linkage disequilibrium tests: what do we gain? European Journal of Human Genetics. 9:291-300; and Zhang, K. et al. (2002). Haplotype block structure and its applications to association studies: power and study designs. American Journal of Human Genetics. 71:1386-1394). This viewpoint is further substantiated by Khoury et al. ((1993). Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for r² of ≧0.5) are potentially useful in predicting the identity of an allele of interest (i.e. associated with the condition of interest). A high degree of linkage disequilibrium may be represented by an absolute value for r² of ≧0.6. Alternatively, a high degree of linkage disequilibrium may be represented by an absolute value for r² of ≧0.7 or by an absolute value for r² of ≧0.8. Additionally, a high degree of linkage disequilibrium may be represented by an absolute value of r² by ≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. For example, in the population from which the haplotype map was created the SNP at position 599 of SEQ. ID NO.: 3 was in “linkage disequilibrium” with position 1826 of SEQ. ID NO.: 4, whereby when the genotype of 599 is T the genotype of 1826 is G. Similarly, when the genotype of 1826 is A the genotype of 599 is C. Accordingly, the determination of the genotype at the 599 locus of SEQ. ID NO.: 3 will provide the identity of the genotype at 1826 or any other locus in “linkage disequilibrium” therewith. Particularly, where such a locus is has a high degree of linkage disequilibrium thereto.

Linkage disequilibrium is useful for genotype-phenotype association studies. If a specific allele at one SNP site (e.g. “A”) is the cause of a specific clinical outcome (e.g. call this clinical outcome “B”) in a genetic association study then, by mathematical inference, any SNP (e.g. “C”) which is in significant linkage disequilibrium with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on linkage disequilibrium between A and C.

Until the mechanism underlying the genetic contribution to a specific clinical outcome is fully understood, linkage disequilibrium helps identify potential candidate causal SNPs and also helps identify a range of SNPs that may be clinically useful for prognosis of clinical outcome or of treatment effect. If one SNP within a gene is found to be associated with a specific clinical outcome, then other SNPs in linkage disequilibrium will also have some degree of association and therefore some degree of prognostic usefulness. For to example, we tested multiple SNPs, having a range of linkage disequilibrium with F3 SNP 599, for individual association with 28 day survival in our SIRS/sepsis cohort of ICU patients. We ordered the SNPs by the degree of linkage disequilibrium with F3 599. We found, as expected from the above discussion, that SNPs with high degrees of linkage disequilibrium with F3 599 also had high degrees of association with this specific clinical outcome. As linkage disequilibrium decreased, the degree of association of the SNP with 28 day survival also decreased. These data support the logical conclusion that if A˜B and C˜A, then C˜B. That is, any SNP, whether already discovered or as yet undiscovered, that is in linkage disequilibrium with F3 599 will be a predictor of the same clinical outcomes that F3 599 is a predictor of. The similarity in prediction between this known or unknown SNP and F3 599 will depend on the degree of linkage disequilibrium between this SNP and F3 599.

It will be appreciated by a person of skill in the art that further linked SNP sites could be determined. The haplotype for F3 can be created by assessing the SNPs of the F3 sequence in normal subjects using a program that has an expectation maximization algorithm (for example PHASE; Stephens M and Donnelly P, 2003, American Journal of Human Genetics 73:1162-1169). A constructed haplotype of F3 may be used to find combinations of SNPs that are in linkage disequilibrium with position 599 or position 1826 of SEQ ID NO:3, 4. Therefore, the haplotype of an individual could be determined by genotyping other SNPs that are in LD with position 599 or position 1826 or 1089 or 4524 or 13925 of SEQ ID NO:1-5. Linked single polymorphism sites or combined polymorphism sites could also be genotyped for assessing subject prognosis.

Numerous sites have been identified as polymorphic sites in the tissue factor gene (see TABLE 1A). Furthermore, the polymorphisms in TABLE 1A are linked to (in linkage disequilibrium with) numerous polymorphism as set out in TABLE 1B below and may also therefore be indicative of subject prognosis.

TABLE 1A
Polymorphisms in the coagulation factor III (F3) gene genotyped in a cohort
of critically ill subjects who had sepsis or SIRS or septic shock. Minor Allele Frequencies
(MAFs) for Caucasians were taken from http://pga.gs.washington.edu/ (The Seattle SNPs
PGA website).

| | | | May 2004 | | | |
|---|---|---|---|---|---|---|
| Polymorphism Name | | | Chromosomal | Seattle | | Minor |
| Chromosomal | Official Gene | | position | SNPs | Minor | Allele |
| position. Alleles | Name | rs# | (Build 35) | position | allele | Frequency |
| F3.599.C/T | coagulation | rs958587 | 94721166 | 599 | C | 0.48 |
| | factor 3 (F3) | | | | | |
| F3.1089.A/G | coagulation | rs3761955 | 94720676 | 1089 | A | 0.48 |
| | factor 3 (F3) | | | | | |
| F3.1826.A/G | coagulation | rs1361600 | 94719939 | 1826 | A | 0.48 |
| | factor 3 (F3)) | | | | | |
| F3.4524.T/C | coagulation | rs696619 | 94717241 | 4524 | T | 0.38 |
| | factor 3 (F3)) | | | | | |
| F3.13925.T/C | coagulation | rs3354 | 94707840 | 13295 | T | 0.22 |
| | factor 3 (F3)) | | | | | |

It will be appreciated by a person of skill in the art, that the numerical designations of the positions of polymorphisms within a sequence are relative to a specific sequence and that the same positions may be assigned different numerical designations depending on the way in which the sequence is numbered and the sequence chosen, as illustrated by the alternative numbering of equivalent polymorphisms DONAHUE B S. et al. and ARNAUD E. et al. above. Furthermore, sequence variations within the population, such as insertions or deletions, may change the relative position and subsequently the numerical designations of particular nucleotides at and around a polymorphism site. Please note that where allele designations differ from the alleles identified in the priority applications, these SNPs were genotyped on the complementary strand and accordingly the designations given are the compliments of the allele designations given herein. For example, 1089 is identified in some places as being T/C in the priority applications, based on genotyping of the complementary strand, but is identified herein as A/G. Accordingly, it would also be appreciated by a person of skill in the art that genotyping the complimentary strand will also provide allele information which may be used to determine patient outcome or to predict patient response to activated protein C or protein C like compound administration (an anti-inflammatory agent or an anti-coagulant agent). The allele designations given below in TABLE 1B relate to the “rs” designated alleles.

TABLE 1B
Polymorphisms in linkage disequilibrium with those listed in TABLE 1A
above, as identified using the Haploview program (BARRETT JC. et al. Bioinformatics
(2005) 21(2): 263-5 (http://www.broad.mit.edu/mpg/haploview/)). Linkage Disequilibrium
between markers was defined using the r² coefficient (as an alternative a D prime (D′)
coefficient may be used), whereby all SNPs available on Hapmap.org (phase I) across a
100 kb area around our genes of interest were included. A minimum r² of 0.5 was used as
the cutoff to identify LD SNPs. The genes are identified, along with the alleles, rs
designations.

| | | | | | | | Chromosomal |
|---|---|---|---|---|---|---|---|
| | | Survival | | | | rsIDs of | Position |
| | Tag | Association | Polymorphism | Polymorphisms | LD | Polymorphisms | (NCBI Build |
| GENE | Polymorphisms | Alleles | rsID | in LD | Alleles | in LD | 35; May 2005) |
| F3 | 599 | T | rs958587 | 4524 | C | rs696619 | 94721166 |
| | | | | 5660 | G | rs762485 | 94716105 |
| | | | | 5730 | C | rs841697 | 94716035 |
| | | | | 7533 | T | rs1144300 | 94714232 |
| | | | | 7754 | A | rs3917615 | 94714011 |
| | | | | 10224 | C | rs2794470 | 94711541 |
| | | | | 10247 | A | rs841695 | 94711518 |
| F3 | 1089 | A | rs3761955 | 599 | T | rs958587 | 94721166 |
| | | | | 1826 | G | rs1361600 | 94719939 |
| | | | | 4524 | C | rs696619 | 94721166 |
| | | | | 5660 | G | rs762485 | 94716105 |
| | | | | 5730 | C | rs841697 | 94716035 |
| | | | | 7533 | T | rs1144300 | 94714232 |
| | | | | 7754 | A | rs3917615 | 94714011 |
| F3 | 1826 | G | rs1361600 | 599 | T | rs958587 | 94721166 |
| | | | | 1089 | A | rs3761955 | 94719939 |
| | | | | 4524 | C | rs696619 | 94721166 |
| | | | | 5660 | G | rs762485 | 94716105 |
| | | | | 5730 | C | rs841697 | 94716035 |
| | | | | 7533 | T | rs1144300 | 94714232 |
| | | | | 7754 | A | rs3917615 | 94714011 |
| | | | | 10224 | C | rs2794470 | 94711541 |
| | | | | 10247 | A | rs841695 | 94711518 |
| F3 | 4524 | T | rs696619 | 599 | C | rs958587 | 94721166 |
| | | | | 1089 | G | rs3761955 | 94720676 |
| | | | | 1826 | A | rs1361600 | 94719939 |
| | | | | 5334 | G | rs762484 | 94716431 |
| | | | | 5660 | T | rs762485 | 94716105 |
| | | | | 5730 | T | rs841697 | 94716035 |
| F3 | 13925 | C | rs3354 | 10085 | G | rs841696 | 94711680 |
| | | | | 11990 | — | rs3917628 | 94709776 |
| | | | | 11993 | — | rs3917629 | 94709773 |
| | | | | 14330 | C | rs841691 | 94707432 |

It will be appreciated by a person of skill in the art that further linked polymorphic sites and combined polymorphic sites may be determined. The haplotype of the F3 gene can be created by assessing polymorphisms in the F3 gene in normal subjects using a program that has an expectation maximization algorithm (i.e. PHASE). A constructed haplotype of the F3 gene may be used to find combinations of SNP's that are in linkage disequilibrium (LD) with the haplotype tagged SNPs (htSNPs) identified herein. Accordingly, the haplotype of an individual could be determined by genotyping other SNPs or other polymorphisms that are in LD with the htSNPs identified herein.

It will be appreciated by a person of skill in the art, that the numerical designations of the positions of polymorphisms within a sequence are relative to the specific sequence. Also the same positions may be assigned different numerical designations depending on the way in which the sequence is numbered and the sequence chosen, as illustrated by the alternative numbering of the equivalent polymorphism (rs958587), whereby the same polymorphism identified C/T at position 599 of the GenBank sequence AF540377 (SEQ ID NO:17), which corresponds to position 301 of SEQ ID NO:3 and to position −1812 in ARNAUD E. et al. (Arterioscler Thromb Vasc Biol (2000) 20:892-898). Furthermore, sequence variations within the population, such as insertions or deletions, may change the relative position and subsequently the numerical designations of particular nucleotides at and around a polymorphic site.

Polymorphic sites in SEQ ID NO:1-5 and SEQ ID NO:6-16 are identified by their variant designation (i.e. M, W, Y, S, R, K, V, B, D, H or by “−” for a deletion, a “+” or “G” etc. for an insertion).

An “rs” prefix designates a SNP in the database is found at the NCBI SNP database (http://www.ncbi.nlm.nih.gov/entrez/query.fcgi?db=Snp). The “rs” numbers are the NCBI|rsSNP ID form.

TABLE 1C

below shows the flanking sequences for a
selection of coagulation factor III (F3)
gene SNPs providing their rs designations,
alleles and corresponding SEQ ID NO
designations. Each polymorphism is at
position 201 within the flanking sequence,
(unless otherwise indicated), and identified
in bold and underlined.

| | | SEQ | |
|---|---|---|---|
| | | ID | |
| GENE | SNP | NO: | FLANKING SEQUENCE |
| F3 | rs3354 | 1 | TATATTATAGACATATGTTAGAAAAGTCCTA |
| 13925 | at | | GAAATGCACCCAATTTCCTTCCATTTTACTT |
| C/T | position | | TCCTACATGGATTGAAGTCAGCCCCTCAAAA |
| | 569 | | GCTTTTCGGCTGGGCATGGTGGTTCACGCCC |
| | | | ATAATACTAGCACTTTGGGAGGCCAAGGTGG |
| | | | GTGGATCAACTGAGGTCAGGAATTCAAGACC |
| | | | AGCCTGGCCAAGATGGTGAAACCCCATCTCT |
| | | | ACTAAAAAATACAAAAATTAGCTTGGTGTGG |
| | | | TAGTGCGCACCTGTAATCCCAGCTACTCGGG |
| | | | AGGCTGAGGCAGACAATTGCTTGAACCCGAG |
| | | | AGACGGAGGGTGCAGTGAGCCGAGATCGTGC |
| | | | TACTGCACTCCAGCCTGGGCAACAGAGCAAG |
| | | | ACTCCGTCTCAAAAAAAAAAAAAAAAAAAAA |
| | | | GCTTTTCAAAAGTCCACCCAGGATTTTTTAA |
| | | | GACATTTTCCCATTTGTTTTTGCTTGGACGA |
| | | | CCTGGTTACTCCTTGAGTGCGGAATATATAA |
| | | | TCTAAAGCATGTTATGTGCAAAGGTGCCATG |
| | | | GTGTTAAAAAATTAAAACTTGGAATTGGTTG |
| | | | TAGTACCATTYGTTACATTTCAAAGTGACTA |
| | | | ATGCTGATGTCAAAACCAGAATGCTAATGGT |
| | | | AATAACAGGTCATATCAAGAGTTTTTTGAAC |
| | | | TCCAGGGTCTTCATGCTCCGAAATACTCATT |
| | | | TGCGTTTCCATGTATTCTATCCTCTTAAAAG |
| | | | TTCTCGGTCACAGTGCAATATAGCATTTGCA |
| | | | GTAGCTCCAACAGTGCTTCCTTTAT |
| F3 | rs696619 | 2 | GTGTGCTTTGGGTCATGATAGATTAATTAAT |
| 4524 | at | | CTCATCTAAACATTGATGTCTTTTTCTGTTG |
| C/T | position | | CTGTCTAGACTGTGAACAATGTCTAACACCT |
| | 301 | | TAGGGAAGAGGTGGGGAGGAATCCCAATGTA |
| | | | TACATTGCCCTTAAGCAGTGTTTGATTCATT |
| | | | CATCTTTGGACTCCATGAATCGAAATCTGGT |
| | | | AGAATACATGATCTTAGTGGAGGAGGCCAAA |
| | | | TGCGTGACTCACTGAGCCTGGCAGAGCAGAA |
| | | | ATACTCTGCTGTCTGCACCCTCTGGGTCTGG |
| | | | TGTGGCTCTGCTTCTTGGTGCYTCAACTCTG |
| | | | ACTGGCAGCTGTCCCCAGGAGGCGATAATTC |
| | | | AGCATGTTCAATCTAAAGGTTATGACTTCCT |
| | | | TGATGGTTTTCACCATATTCTTGGCAAGTTT |
| | | | TTGGTTTTTGAAATGTTCTAGGAGGCTTGGT |
| | | | AGAGATCTTATGAAATAGAGAATAGCTGCTG |
| | | | TGGAAATTATTTTAATGCTAATTACATAAAA |
| | | | GTACAAAAGTAGCACTAGCTAAAACAAAAGG |
| | | | TATTTTGCTGTTCTGTTTTGTTTTAGCTTGT |
| | | | GCCAGGCCTTTTACAGCATTAGGAATGCAAC |
| | | | TTCTAGATAACG |
| F3 | rs958587 | 3 | ACTGCTAAGCTTGACTTTACTGACAGGAGTA |
| 599 | at | | AAAAAAATTGTGTTAAGGTTAGGGAATAATT |
| C/T | position | | TTAACAGTCAATTTGTTCTTGTGAACAAATT |
| | 301 | | TCAACAGTGAAATTTTAGATATGTACTTTTT |
| | | | AATGGTGCCAAGCAGCAGTTATTATAGATCA |
| | | | ACTGCTGTTTGGCACCATTAAAAAGTACACT |
| | | | TCGCACCGTCAAAAAGTAGATCTGGCCACAA |
| | | | TTAGATCAGTCAGGGAAAAACACTTCGCAAT |
| | | | GAAATATTATTTACCACGTTTTCTTCCTCCC |
| | | | TCTTCTTGAAAATAGTAATGAYTTTAGCATT |
| | | | TTTAAATCTTGAAGAATGTCATTCCGTACTG |
| | | | ACTAAAAAGCCTGTGCAAACACCCAACATCT |
| | | | TCTCTTTCCTGTCTATTTTAATGGATATACA |
| | | | AAATAAATATTCATCTAATTTATCAATATTT |
| | | | AAGGCACATTGTGTATCACAATCCAATGGTA |
| | | | AACATTTTTCTTATCAAATGGCTGATCTTGA |
| | | | CATTGTTTAAAGCCCTATATGATTTCAGAAA |
| | | | AGTCAGTGACTCCAAGTGGAATTGCAACAAT |
| | | | TTCTTGGGTCTTGTAACAAACCTGAAGTGTA |
| | | | ACTATTTCTCTT |
| F3 | rs1361600 | 4 | TGTCTTCTTCAATGTGGGAACCATCCTTTCT |
| 1826 | at | | GGAACCACAGAGCTGCAGATGTCACGCTGGA |
| A/G | position | | ATTCTCCCAGAGGCAAACTGCCAGATGTGAG |
| | 256 | | GCTGCTCTTCCTCAGTCACTATCTCTGGTCG |
| | | | TACCGGGCGATGCCTGAGCCAACTGACCCTC |
| | | | AGACCTGTGAGCCGAGCCGGTCACACCGTGG |
| | | | CTGACACCGGCATTCCCACCGCCTTTCTCCT |
| | | | GTGCGACCCGCTAAGGGCCCCGCGAGGTGGG |
| | | | CAGGCCARGTATTCTTGACCTTCGTGGGGTA |
| | | | GAAGAAGCCACCGTGGCTGGGAGAGGGCCCT |
| | | | GCTCACAGCCACACGTTTACTTCGCTGCAGG |
| | | | TCCCGAGCTTCTGCCCCAGGTGGGCAAAGCA |
| | | | TCCGGGAAATGCCCTCCGCTGCCCGAGGGGA |
| | | | GCCCAGAGCCCGTGCTTTCTATTAAATGTTG |
| | | | TAAATGCCGCCTCTCCCACTTTATCACCAAA |
| | | | TGGAAGGGAAGAATTCTTCCAAGGCGCCCTC |
| | | | CCTTTCCTGCCATAG |
| F3 | rs3761955 | 5 | TTACTCTATATTGCCTGCAAGACACGACTGG |
| 1089 | at | | AGAATTCTTAGTGTAAGAATTGTTTGTTTCC |
| A/G | position | | TCTCTCCTTCTTTCCCACGTTTTCCCAGGGA |
| | 256 | | AGTCAGTCTTGCATTTTAATGCATACTATAT |
| | | | ACATATCTCGTTTAGCTTACTGAACCACTTG |
| | | | TTTTAACAGAATAAAACTGTGCAAAATTTTA |
| | | | ATTTTCCTCCTTTGCCTGAACTGAAATAGCA |
| | | | CATCCAGGTTTAGCCCTTGTAGACTTTCCTT |
| | | | CCTCGAARCAGAAAGTTGCCCTTGATGATTT |
| | | | CCTCTTTGAGCTCTCTGCCAGCTCTGAAACC |
| | | | CACAAAATTTATGTTTGCAAAACTAAGCCAT |
| | | | GCAATCCTCTTTTTATGCAGGCTCTAGCCTG |
| | | | AGTCATTTTCCCTAAGAGATCTTCAGCTCCA |
| | | | CCTGGGATGTGATTCTTTGCTCTCTGGGATT |
| | | | GAAGGTAGCTGAAGAGAAATAGTTACACTTC |
| | | | AGGTTTGTTACAAGACCCAAGAAATTGTTGC |
| | | | AATTCCACTTGGAGT |

The Sequences given in TABLE 1C (SEQ ID NO:1-5) above and in TABLE 1D (SEQ ID NO:6-16) would be useful to a person of skill in the art in the design of primers and probes or other oligonucleotides or peptide nucleic acids for the identification of factor III gene SNP alleles and or genotypes as described herein.

TABLE 1D

below shows the flanking sequences for a
selection of coagulation factor III (F3)
gene SNPs in LD with the tagged SNPs in
TABLE 1C, providing their rs designations,
alleles and corresponding SEQ ID NO
designations. However, where a SNP in LD is
also an htSNP it only occurs in TABLE 1C
above. Each SNP is at position 201 of the
flanking sequence (unless otherwise
indicated) and identified in bold and
underlined.

| | | | |
|---|---|---|---|
| | | SEQ | |
| | | ID | |
| GENE | SNP | NO: | FLANKING SEQUENCE |

| | | | |
|---|---|---|---|
| F3 | rs762484 | 6 | TTCAGATTTCACCAATTGAGAATTAGTAA |
| 5334 | at | | GTAATTTCTCTGATACAGGCCTGAAGTTT |
| A/G | position | | ACCTTAGTAAACACTTTACTTCCATATGG |
| | 322 | | TAAAAATTAGATTTTGGGAGGAATGCTTA |
| | | | CCTCCTAAATATATTCAATCTAATATTTG |
| | | | AGGACACATGGGAATATATTTATGATTCA |
| | | | TCTGCTTTTTAAACATAAGCCTTTGTTAA |
| | | | CTGTAAGTTCTTGAACTTTATAAGGCTGC |
| | | | TGTTATTTAAATGAGCACAGCTCCTGATC |
| | | | TGCAAACAGCAGAGCGCAGGGCTACAGCT |
| | | | TGGGGGATGCCAGCCGACTCAGGGTGGTC |
| | | | CTRTGGACTGAACAATCTCTTGCTGCTGT |
| | | | ACTGGAGGGCCTGGGAGCTTTTCCATCAG |
| | | | CCTCGGCCTGAGGTGTGCACTCTTCTCCT |
| | | | GCCCACCCCAGGAATAAATGAGATTCCTG |
| | | | GTTAAAAAGGACCAGAGCAGTCATTTTAC |
| | | | AGTTGAGGAAACTGTTGCTCTGAGAAGTG |
| | | | AGGGATTTATTCATGACTACACTGATGGT |
| | | | GAGTGCCCATGTCAGGTCTGGAACCAAAG |
| | | | TCTACCCAGTATCCACACACCACCATCCC |
| | | | TCAGGTGGCTCTGCCACAGTCTGATGGGA |
| | | | GGCTCCAAAGCGGGAGGAAGAAGGAAAGT |
| | | | CTTGCCCACTGCATCTCCTCAGTTGGCCT |
| | | | TCCTCTCTGCCTGTTTTCCCTCCCTACAG |
| | | | TTAGCATCTTAAGCA |
| F3 | rs762485 | 7 | TTCAGATTTCACCAATTGAGAATTAGTAA |
| 5660 | at | | GTAATTTCTCTGATACAGGCCTGAAGTTT |
| G/T | position | | ACCTTAGTAAACACTTTACTTCCATATGG |
| | 648 | | TAAAAATTAGATTTTGGGAGGAATGCTTA |
| | | | CCTCCTAAATATATTCAATCTAATATTTG |
| | | | AGGACACATGGGAATATATTTATGATTCA |
| | | | TCTGCTTTTTAAACATAAGCCTTTGTTAA |
| | | | CTGTAAGTTCTTGAACTTTATAAGGCTGC |
| | | | TGTTATTTAAATGAGCACAGCTCCTGATC |
| | | | TGCAAACAGCAGAGCGCAGGGCTACAGCT |
| | | | TGGGGGATGCCAGCCGACTCAGGGTGGTC |
| | | | CTGTGGACTGAACAATCTCTTGCTGCTGT |
| | | | ACTGGAGGGCCTGGGAGCTTTTCCATCAG |
| | | | CCTCGGCCTGAGGTGTGCACTCTTCTCCT |
| | | | GCCCACCCCAGGAATAAATGAGATTCCTG |
| | | | GTTAAAAAGGACCAGAGCAGTCATTTTAC |
| | | | AGTTGAGGAAACTGTTGCTCTGAGAAGTG |
| | | | AGGGATTTATTCATGACTACACTGATGGT |
| | | | GAGTGCCCATGTCAGGTCTGGAACCAAAG |
| | | | TCTACCCAGTATCCACACACCACCATCCC |
| | | | TCAGGTGGCTCTGCCACAGTCTGATGGGA |
| | | | GGCTCCAAAGCGGGAGGAAGAAGGAAAGT |
| | | | CTTGCCCACKGCATCTCCTCAGTTGGCCT |
| | | | TCCTCTCTGCCTGTTTTCCCTCCCTACAG |
| | | | TTAGCATCTTAAGCA |
| F3 | rs841691 | 8 | CAGAATACCAATGTCTCCTGCACTTAACA |
| 14330 | at | | CATTAATACAAAGTTTGCCAATTGTTTTG |
| A/C | position | | AATTTCCAAATGTATTCCTGAAAAAAAAA |
| | 264 | | AGAACCTAAACACTATATTATAGACATAT |
| | | | GTTAGAAAAGTCCTAGAAATGCACCCAAT |
| | | | TTCCTTCCATTTTACTTTCCTACATGGAT |
| | | | TGAAGTCAGCCCCTCAAAAGCTTTTCGGC |
| | | | TGGGCATGGTGGTTCACGCCCATAATACT |
| | | | AGCACTTTGGGAGGCCAAGGTGGGTGGAT |
| | | | CAMCTGAGGTCAGGAATTCAAGACCAGCC |
| | | | TGGCCAAGATGGTGAAACCCCATCTCTAC |
| | | | TAAAAAATACAAAAATTAGCTTGGCGTGG |
| | | | TAGTGCGCACCTGTAATCCCAGCTACTCG |
| | | | GGAGGCTGAGGCAGACAATTGCTTGAACC |
| | | | CGAGAGACGGAGGGTGCAGTGAGCCGAGA |
| | | | TCGTGCTACTGCACTCCAGCCTGGGCAAC |
| | | | AGAGCAAGACTCCGTCTCAAAAAAAAAAA |
| | | | AAAAAAAAAAAAAGCTTTTCAAAAGTCCA |
| | | | CCCAGGATTTTTTAAGACATTTTCCCATT |
| | | | TGTTTTTGCTTGGACGACCTGGTTACTCC |
| | | | TTGAGTGCGGAATATATAATCTAAAGCAT |
| | | | GTTATGTGCAAAAGGTGCCATGGTGTTAA |
| | | | AAATTAAAACTTGGAATTGGTTGTAGTAC |
| | | | CATTCGTTACATTTCAAAGTGACTAATGC |
| | | | TGATGTCAAA |
| F3 | rs841695 | 9 | TTGTTGGCTGTCCGAGGTTTGCTGAAACA |
| 10247 | at | | AAGGAAATGAGCTTGGTTGGAACCAAAGA |
| A/G | position | | ATTCTGTACAAAGTCAAATCCTGTTTTGT |
| | 694 | | TATCACAATTGACAACTTAATTATCTCTC |
| | | | ATATAAAACATGTGCATAGAACCAGCTCC |
| | | | CTGAAAGAAGCAGGCGTGGCGGCCTGGAG |
| | | | CTGAATCCTAAGACATTCTGTGGTAGTGC |
| | | | TGGCCCAAGGGGAAGACAATGGAGCCTCA |
| | | | GATGTCATTTTAAGTTTTCTAGTAGTCAC |
| | | | ATTAGAAAAAGTAAAAAGGAACAGGTGAA |
| | | | ATTAATTTTAATAATATATTTTATTTAAC |
| | | | CAAAGACAGTTGACCCCTGAACAACATGG |
| | | | GTTTGAACTCTGTGGATCCACTTGTATGC |
| | | | AAATTTTTTTAAATAAAAGTTACACCAAG |
| | | | TGTGCCGCCTCCTACTTCCTCCATCTTTT |
| | | | CTCCTGCCGTGGCCCCTGCTCTTCCTCCT |
| | | | CCTCCTCCTCCTCAGCCTACTCAACATGA |
| | | | AGATGACGAGGATGAACACCTTTATGATG |
| | | | ATCCACTTCCACTTTATCAATAGTAAATA |
| | | | TATTTTCTCTTCCTTATAATTCTTTCTCT |
| | | | TCCTTCCTTCTTTCTTTTCTTTTCTTTTC |
| | | | TTTTTTTTCTTTCTTTCCCTTTCTTTTTT |
| | | | AGACAGAGTCTCGCTCTGTCACCCAGGCC |
| | | | GGAGTGCAGTGGCGCAATCTCAGTTCRCT |
| | | | GCAACCTCCTCCACCTGGGTTGAAGTGAT |
| | | | TCTCCTGCCTCAGCCTCCCAAGTAGCTGG |
| | | | GATTACAGGCACCCACAACCACGCCTGGC |
| | | | TAATTTTTGTATTTTTAGTAGAGATGGGG |
| | | | TTTCACCATGTTGGCCAGGCTGGTCTTGA |
| | | | ACTCCTGACCTCAAGTGATCCACCCGCCT |
| | | | TAGCATCCCAAAGTTCTGAGATTACAGGC |
| | | | ACGAGCCACCATGCCCAGCCTCTTTTCCT |
| | | | TATAATTTTCTTAATAACATTTTCTTTCC |
| | | | TCTAGCTTACTTTATTGTAAGAATACAGT |
| | | | ATATAATACACGCAACATGAAAAATGTGT |
| | | | GTTAATTGACTGTTTATCTTATTGGTAAG |
| | | | GCTGCAGTCAACAGTCAACCGATAGGTAG |
| | | | GCTATTAGTAGTTAAGTTTTTGGGGAGTC |
| | | | AAGTTATACTCGGATTTTCAACTGCACAG |
| | | | GGAGTCGGTGCCTCTCAGCCATGCATTGC |
| | | | TTGAGTCAACTGCATATATCTGGAATATA |
| | | | ATCATTTTAATGTGTAATCAATATAAAAA |
| | | | AGTTTTGAGATATTCTACACTTCTTAAAT |
| | | | TCCAGTGTGTACTTTATACTTAGAGCATT |
| | | | AGCCATATTTCAAGTGGTCAACAGCCACA |
| | | | TGTGGCTAGTGGCTACTATACTGAACAGG |
| | | | GTGGTAGCATAAACTACGATATTTTAGGT |
| | | | TCAAAAATATGTAGGGCTGTGAGAAAGCC |
| | | | CAGGAAATGTCCTTGGTACCTCGAGAAGC |
| | | | CGGATTTAACCACGGGGTCTCTGCAATGG |
| | | | CTCAATTATCTCATGTGCTTTGGTTAAAT |
| | | | GACAGGAAAACAAACATCAATTCACAGGC |
| | | | CAAATATGTGTGTGCACATGCAAA |
| F3 | rs841696 | 10 | TTGTTGGCTGTCCGAGGTTTGCTGAAACA |
| 10085 | at | | AAGGAAATGAGCTTGGTTGGAACCAAAGA |
| A/G | position | | ATTCTGTACAAAGTCAAATCCTGTTTTGT |
| | 856 | | TATCACAATTGACAACTTAATTATCTCTC |
| | | | ATATAAAACATGTGCATAGAACCAGCTCC |
| | | | CTGAAAGAAGCAGGCGTGGCGGCCTGGAG |
| | | | CTGAATCCTAAGACATTCTGTGGTAGTGC |
| | | | TGGCCCAAGGGGAAGACAATGGAGCCTCA |
| | | | GATGTCATTTTAAGTTTTCTAGTAGTCAC |
| | | | ATTAGAAAAAGTAAAAAGGAACAGGTGAA |
| | | | ATTAATTTTAATAATATATTTTATTTAAC |
| | | | CAAAGACAGTTGACCCCTGAACAACATGG |
| | | | GTTTGAACTCTGTGGATCCACTTGTATGC |
| | | | AAATTTTTTTAAATAAAAGTTACACCAAG |
| | | | TGTGCCGCCTCCTACTTCCTCCATCTTTT |
| | | | CTCCTGCCGTGGCCCCTGCTCTTCCTCCT |
| | | | CCTCCTCCTCCTCAGCCTACTCAACATGA |
| | | | AGATGACGAGGATGAACACCTTTATGATG |
| | | | ATCCACTTCCACTTTATCAATAGTAAATA |
| | | | TATTTTCTCTTCCTTATAATTCTTTCTCT |
| | | | TCCTTCCTTCTTTCTTTTCTTTTCTTTTC |
| | | | TTTTTTTTCTTTCTTTCCCTTTCTTTTTT |
| | | | AGACAGAGTCTCGCTCTGTCACCCAGGCC |
| | | | GGAGTGCAGTGGCGCAATCTCAGTTCGCT |
| | | | GCAACCTCCTCCACCTGGGTTGAAGTGAT |
| | | | TCTCCTGCCTCAGCCTCCCAAGTAGCTGG |
| | | | GATTACAGGCACCCACAACCACGCCTGGC |
| | | | TAATTTTTGTATTTTTAGTAGAGATGGGG |
| | | | TTTCACCATGTTGGCCAGGCTGGTCTTGA |
| | | | ACTCCTGACCTCAARTGATCCACCCGCCT |
| | | | TAGCATCCCAAAGTTCTGAGATTACAGGC |
| | | | ACGAGCCACCATGCCCAGCCTCTTTTCCT |
| | | | TATAATTTTCTTAATAACATTTTCTTTCC |
| | | | TCTAGCTTACTTTATTGTAAGAATACAGT |
| | | | ATATAATACACGCAACATGAAAAATGTGT |
| | | | GTTAATTGACTGTTTATCTTATTGGTAAG |
| | | | GCTGCAGTCAACAGTCAACCGATAGGTAG |
| | | | GCTATTAGTAGTTAAGTTTTTGGGGAGTC |
| | | | AAGTTATACTCGGATTTTCAACTGCACAG |
| | | | GGAGTCGGTGCCTCTCAGCCATGCATTGC |
| | | | TTGAGTCAACTGCATATATCTGGAATATA |
| | | | ATCATTTTAATGTGTAATCAATATAAAAA |
| | | | AGTTTTGAGATATTCTACACTTCTTAAAT |
| | | | TCCAGTGTGTACTTTATACTTAGAGCATT |
| | | | AGCCATATTTCAAGTGGTCAACAGCCACA |
| | | | TGTGGCTAGTGGCTACTATACTGAACAGG |
| | | | GTGGTAGCATAAACTACGATATTTTAGGT |
| | | | TCAAAAATATGTAGGGCTGTGAGAAAGCC |
| | | | CAGGAAATGTCCTTGGTACCTGGAGAAGC |
| | | | CGGATTTAACCACGGGGTCTCTGCAATGG |
| | | | CTCAATTATCTCATGTGCTTTGGTTAAAT |
| | | | GACAGGAAAACAAACATCAATTCACAGGC |
| | | | CAAATATGTGTGTGCACATGCAAA |
| F3 | rs841697 | 11 | CAAAACCCTCTGATTTGGAATTTTGAGTT |
| 5730 | at | | AACTAAAAAATTCAGTCACTAATTTGGTT |
| A/G | position | | GCAGGTTGTTTTCCAGAAGCTTTGTAAAT |
| | 301 | | TCAGCTTTAGAATTCAGAACATTTCCATG |
| | | | GAATGAATATCACCGGTGACGGTTTGTGC |
| | | | TAAGGCTTAAGCCAATAACATTTCCCAAC |
| | | | CACCACTGAAAACTGTTAGCAAAGGTGAA |
| | | | AAATGCAGTTGGAGTTCCAAGTAGGGGCT |
| | | | TCTGCACAGCAGTAGTGTCCTGCGGCTGG |
| | | | AGCCAGGCTGCAGTAGTGAGAGCAGTCGG |
| | | | GAGGGAAGAGRGGCAGCTGCTTAAGATGC |
| | | | TAACTGTAGGGAGGGAAAACAGGCAGAGA |
| | | | GGAAGGCCAACTGAGGAGATGCAGTGGGC |
| | | | AAGACTTTCCTTCTTCCTCCCGCTTTGGA |
| | | | GCCTCCCATCAGACTGTGGCAGAGCCACC |
| | | | TGAGGGATGGTGGTGTGTGGATACTGGGT |
| | | | AGACTTTGGTTCCAGACCTGACATGGGCA |
| | | | CTCACCATCAGTGTAGTCATGAATAAATC |
| | | | CCTCACTTCTCAGAGCAACAGTTTCCTCA |
| | | | ACTGTAAAATGACTGCTCTGGTCCTTTTT |
| | | | AACCAGGAATCTCATTTATTC |
| F3 | rs1144300 | 12 | TTAGACAGATACTACCTGTACTCTTATTC |
| 7533 | at | | TGTAATCTTTGTTGGGATGGATTCACATC |
| C/T | position | | TTGCAAAGGAAGGGAGGCATGTAGTATAA |
| | 478 | | TGGGGCAAACAGACCCAGCTCTGCCACTC |
| | | | GTTAGATATGTGACCTTCTGCAAGTTGCT |
| | | | TAGTGCCTGTGAGCTTCAGTGTCCTCATG |
| | | | GATAAGAAAGATCCAACACCTTCTTGGAA |
| | | | GGATTATATCAAATGAAGTAACATGAGTA |
| | | | AAGGGTCCAGCAGAATACCTGGCATATAG |
| | | | TGGAGTCAATGAATGATTAATAATATTAT |
| | | | TAATAGTGGTCATGAGAGAATATATGTAT |
| | | | AACATGTTATTATGTAGACTCACTATATA |
| | | | GACTCTATTCTACATAGAATATAGAACAT |
| | | | TATATAACAAACAACTATAATAAGTAGAC |
| | | | TATAGTAAACAACCTCACTTTGTCTCAGT |
| | | | TGCCTCATCTTGATGGAAAACTGCTCTTT |
| | | | CTCTCCTGTTACCYTGACAGAGAGCGTCT |
| | | | ACATTCTAAAAGAAAGATATTTAACAAAA |
| | | | TGGTTGAGTACAGATCCAAGAGTCAAATA |
| | | | GCTGTCTGGTTCAAAGTCCAGCTGTGTGA |
| | | | TTTTGAGCTAGTCACCCAATCTCACTTTG |
| | | | TCTCAGTAGCCTTATTTGTAAAAACAAGG |
| | | | CAAATTACAGAGCCATCCCCTGGGTTGCT |
| | | | ATGAGGACTCAAACATGCATCCCAAGTGC |
| | | | TCGGTGTTGCTAGGTATGATGGCTCACAC |
| | | | CTGTACATTCAGCACTTTGGGAGGCCGAA |
| | | | GCAGAAGGATCAGCCTGGGCAACATAGCA |
| | | | GGACCCCATCTCTACAAAACAATGTTTAA |
| | | | AAAAAAGCAAAGTGCTCAGCACAGTGACT |
| | | | GCATCATTAGGATTGATTGTAGGGCTCCT |
| | | | GATGTTAGCACAGAACACCACAGCCAGGA |
| | | | AGCAGTCTATCTTGTTGGGTGCAAATTGT |
| | | | AACATTCCATTTATGTTTCTTCCTTCTTT |
| F3 | rs3917629 | 13 | TTGATTGATGTGGATAAAGGAGAAAACTA |
| 11993-/ | at | | CTGTTTCAGTGTTCAAGCAGTGATTCCCT |
| G/TG | position | | CCCGAACAGTTAACCGGAAGAGTACAGAC |
| | 256 | | AGCCCGGTAGAGTGTATGGGCCAGGAGAA |
| | | | AGGGGAATTCAGAGGTGAGTGGCTCTGCC |
| | | | AGCCATTTGCCTGGGGGTATGGGTGCTGT |
| | | | GGGTGACTTCTGGAGGAGTAGCTCCACCC |
| | | | TCAGGGCTGGGATATACTTCCTTGGTTAA |
| | | | ATATTCAGGAAAACAAACTGCCC-/G/TG |
| | | | GAGGTTTTTTGTTGTTATTTGTTTGTTTT |
| | | | GGTTTTGATTTTGCTTTGGTACAAAAAAG |
| | | | ATTTTGGACATTTAGAAATGTTTCTGTGT |
| | | | TGATTGTGCCCTTGTATTAGCAGGTGTTT |
| | | | TCTTGAGCACCTGTCATGTGCTAAGCCCT |
| | | | CTGCTGAGCACTGGATACACAAACTGTGT |
| | | | TTAGGATTTAGCAACAAGTCACAGATTTC |
| | | | CCTGGGCATTTTTTCATGCTTAAATTCTA |
| | | | ATTCTGGGGGTGGCTTCTGGACC |
| F3 | rs2794470 | 14 | AGACAATGGAGCCTCAGATGTCATTTTAA |
| 10224 | at | | GTTTTCTAGTAGTCACATTAGAAAAAGTA |
| C/T | position | | AAAAGGAACAGGTGAAATTAATTTTAATA |
| | 501 | | ATATATTTTATTTAACCAAAGACAGTTGA |
| | | | CCCCTGAACAACATGGGTTTGAACTCTGT |
| | | | GGATCCACTTGTATGCAAATTTTTTTAAA |
| | | | TAAAAGTTACACCAAGTGTGCCGCCTCCT |
| | | | ACTTCCTCCATCTTTTCTCCTGCCGTGGC |
| | | | CCCTGCTCTTCCTCCTCCTCCTCCTCCTC |
| | | | AGCCTACTCAACATGAAGATGACGAGGAT |
| | | | GAACACCTTTATGATGATCCACTTCCACT |
| | | | TTATCAATAGTAAATATATTTTCTCTTCC |
| | | | TTATAATTCTTTCTCTTCCTTCCTTCTTT |
| | | | CTTTTCTTTTCTTTTCTTTTTTTTCTTTC |
| | | | TTTCCCTTTCTTTTTTAGACAGAGTCTCG |
| | | | CTCTGTCACCCAGGCCGGAGTGCAGTGGC |
| | | | GCAATCTCAGTTCACTGCAACCTCCTCCA |
| | | | CCTGGGTYGAAGTGATTCTCCTGCCTCAG |
| | | | CCTCCCAAGTAGCTGGGATTACAGGCACC |
| | | | CACAACCACGCCTGGCTAATTTTTGTATT |
| | | | TTTAGTAGAGATGGGGTTTCACCATGTTG |
| | | | GCCAGGCTGGTCTTGAACTCCTGACCTCA |
| | | | AGTGATCCACCCGCCTTAGCATCCCAAAG |
| | | | TTCTGAGATTACAGGCACGAGCCACCATG |
| | | | CCCAGCCTCTTTTCCTTATAATTTTCTTA |
| | | | ATAACATTTTCTTTCCTCTAGCTTACTTT |
| | | | ATTGTAAGAATACAGTATATAATACACGC |
| | | | AACATGAAAAATGTGTGTTAATTGACTGT |
| | | | TTATCTTATTGGTAAGGCTGCAGTCAACA |
| | | | GTCAACCGATAGGTAGGCTATTAGTAGTT |
| | | | AAGTTTTTGGGGAGTCAAGTTATACTCGG |
| | | | ATTTTCAACTGCACAGGGAGTCGGTGCCT |
| | | | CTCAGCCATGCATTGCTTGAGTCAACTGC |
| | | | ATATATCTGGAATATAATCATTTTAATGT |
| | | | GTAATCAATATAAAA |
| F3 | rs3917615 | 15 | CTTGATGGAAAACTGCTCTTTCTCTCCTG |
| 7754 | at | | TTACCTTGACAGAGAGCGTCTACATTCTA |
| A/G | position | | AAGAAAAGATATTTAACAAAATGGTTGAG |
| | 256 | | TACAGATCCAAGAGTCAAATAGCTGTCTG |
| | | | GTTCAAAGTCCAGCTGTGTGATTTTGAGC |
| | | | TAGTCACCCAATCTCACTTTGTCTCAGTA |
| | | | GCCTTATTTGTAAAAACAAGGCAAATTAC |
| | | | AGAGCCATCCCCTGGGTTGCTATGAGGAC |
| | | | TCAAACATGCATCCCAAGTGCTCRGTGTT |
| | | | GCTAGGTATGATGGCTCACACCTGTACAT |
| | | | TCAGCACTTTGGGAGGCCGAAGCAGAAGG |
| | | | ATCAGCCTGGGCAACATAGCAGGACCCCA |
| | | | TCTCTACAAAACAATGTTTAAAAAAAAGC |
| | | | AAAGTGCTCAGCACAGTGACTGCATCATT |
| | | | AGGATTGATTGTAGGGCTCCTGATGTTAG |
| | | | CACAGAACACCACAGCCAGGAAGCAGTCT |
| | | | ATCTTGTTGGGTGCAAATTGTAACATTCC |
| | | | ATTTATGTTTCTTCCTTC |
| F3 | rs3917628 | 16 | GATTGATGTGGATAAAGGAGAAAACTACT |
| 11990-/C | at | | GTTTCAGTGTTCAAGCAGTGATTCCCTCC |
| | position | | CGAACAGTTAACCGGAAGAGTACAGACAG |
| | 251 | | CCCGGTAGAGTGTATGGGCCAGGAGAAAG |
| | | | GGGAATTCAGAGGTGAGTGGCTCTGCCAG |
| | | | CCATTTGCCTGGGGGTATGGGTGCTGTGG |
| | | | GTGACTTCTGGAGGAGTAGCTCCACCCTC |
| | | | AGGGCTGGGATATACTTCCTTGGTTAAAT |
| | | | ATTCAGGAAAACAAACTG-/CCCTGGAGG |
| | | | TTTTTTGTTGTTATTTGTTTGTTTTGGTT |
| | | | TTGATTTTGCTTTGGTACAAAAAAGATTT |
| | | | TGGACATTTAGAAATGTTTCTGTGTTGAT |
| | | | TGTGCCCTTGTATTAGCAGGTGTTTTCTT |
| | | | GAGCACCTGTCATGTGCTAAGCCCTCTGC |
| | | | TGAGCACTGGATACACAAACTGTGTTTAG |
| | | | GATTTAGCAACAAGTCACAGATTTCCCTG |
| | | | GGCATTTTTTCATGCTTAAATTCTAATTC |
| | | | TGGGGGTGGCTTCTG |

The Sequences given in TABLE 1C, 1D and 1E would be useful to a person of skill in the art in the design of primers and probes or other oligonucleotides or peptide nucleic acids for the identification of F3 SNP alleles and or genotypes as described herein.

A representative of a Homo sapiens coagulation factor III (F3) sequence which comprises a sequence as listed in GenBank under accession number AF540377 is found in SEQ ID NO: 17 is found in TABLE 1E below. Polymorphism sites at positions 599, 1089, 1826, 4524 and 13925 of SEQ ID NO:17 are identified in bold. It should be noted that SEQ ID NO:17 as set out below shows 1089 as being Y (C/T) unlike SEQ ID NO:5 in which the same SNP (rs3761955) is identified as R (A/G). Similarly, SEQ ID NO:17 as set out below also shows 13925 as being R (A/G) unlike SEQ ID NO:1 in which the same SNP (rs3354) is identified as Y (C/T). The discrepancy is due to the strand for which sequence is provided. SEQ ID NO:1 and SEQ ID NO:5 show the same SNPs on the complimentary strand to SEQ ID NO:17. Whereas, 599 (rs958587), 1826 (rs1361600), and 4524 (rs696619) of SEQ ID NO:17 are shown on the same strand as their counterpart sequences in SEQ ID NOs:3, 4 and 2 respectively and accordingly have the same SNP allele designations Y (C/T), R (A/G) and Y (C/T) respectively.

TABLE 1E

(SEQ ID NO:17)

| | |
|---|---|
| 1 | taccacccag tcttactttc cttcattggg agtaggagaa agcctggtga tggaaggaaa |
| 61 | gcccccgggg tcagaagacc agatctgcac tgtgtggtta ggcaagccag tttactcaga |
| 121 | tcagctggcc acagggttct catccataaa agaatgtctg tgaggttctc ccatcctctg |
| 181 | acatcctaaa atccaatgag aaagggactg gtcaagccag agagattatt gttatagttt |
| 241 | agtaactttt tgaacttctc agagcctcca agatagatca tggaggaggg aactgttaac |
| 301 | tgctaagctt gactttactg acaggagtaa aaaaaattgt gttaaggtta gggaataatt |
| 361 | ttaacagtca atttgttctt gtgaacaaat ttcaacagtg aaattttaga tatgtacttt |
| 421 | ttaatggtgc caagcagcag ttattataga tcaactgctg tttggcacca ttaaaaagta |
| 481 | cacttcgcac cgtcaaaaag tagatctggc cacaattaga tcagtcaggg aaaaacactt |
| 541 | cgcaatgaaa tattatttac cacgttttct tcctccctct tcttgaaaat agtaatgaYt |
| 601 | ttagcatttt taaatcttga agaatgtcat tccgtactga ctaaaaagcc tgtgcaaaca |
| 661 | cccaacatct tctctttcct gtctatttta atggatatac aaaataaata ttcatctaat |
| 721 | ttatcaatat ttaaggcaca ttgtgtatca caatccaatg gtaaacattt ttcttatcaa |
| 781 | atggctgatc ttgacattgt ttaaagccct atatgatttc agaaaagtca gtgactccaa |
| 841 | gtggaattgc aacaatttct tgggtcttgt aacaaacctg aagtgtaact atttctcttc |
| 901 | agctaccttc aatcccagag agcaaagaat cacatcccag gtggagctga agatctctta |
| 961 | gggaaaatga ctcaggctag agcctgcata aaaagaggat tgcatggctt agttttgcaa |
| 1021 | acataaattt tgtgggtttc agagctggca gagagctcaa agaggaaatc atcaagggca |
| 1081 | actttctgYt tcgaggaagg aaagtctaca agggctaaac ctggatgtgc tatttcagtt |
| 1141 | caggcaaagg aggaaaatta aaattttgca cagttttatt ctgttaaaac aagtggttca |
| 1201 | gtaagctaaa cgagatatgt atatagtatg cattaaaatg caagactgac ttccctggga |
| 1261 | aaacgtggga aagaaggaga gaggaaacaa acaattctta cactaagaat tctccagtcg |
| 1321 | tgtcttgcag gcaatataga gtaataataa taaaaatgac aggagatact ttgacaggat |
| 1381 | ggcttaaaat gccactcaat agatgaagag ttgttctcat gcttggcaag tttacagcaa |
| 1441 | agcccagaag gagaagccag aaaataattt agaaaaacca aagcttaaaa ctagtggcac |
| 1501 | acaactctgg ttacattttt cattttcatt tttggttcct ctgcatttcg gtggaactca |
| 1561 | gtcccacaga tgtcttcttc aatgtgggaa ccatcctttc tggaaccaca gagctgcaga |
| 1621 | tgtcacgctg gaattctccc agaggcaaac tgccagatgt gaggctgctc ttcctcagtc |
| 1681 | actatctctg gtcgtaccgg gcgatgcctg agccaactga ccctcagacc tgtgagccga |
| 1741 | gccggtcaca ccgtggctga caccggcatt cccaccgcct ttctcctgtg cgacccgcta |
| 1801 | agggccccgc gaggtgggca ggccaRgtat tcttgacctt cgtggggtag aagaagccac |
| 1861 | cgtggctggg agagggccct gctcacagcc acacgtttac ttcgctgcag gtcccgagct |
| 1921 | tctgccccag gtgggcaaag catccgggaa atgccctccg ctgcccgagg ggagcccaga |
| 1981 | gcccgtgctt tctattaaat gttgtaaatg ccgcctctcc cactttatca ccaaatggaa |
| 2041 | gggaagaatt cttccaaggc gccctccctt tcctgccata gacctgcaac ccacctaagc |
| 2101 | tgcacgtcgg agtcgcgggc ctgggtgaat ccgggggcct tgggggaccc gggcaactag |
| 2161 | acccgcctgc gtcctccagg gcagctccgc gctcggtggc gcggttgaat cactggggtg |
| 2221 | agtcatccct tgcagggtcc cggagtttcc taccgggagg aggcggggca ggggtgtgga |
| 2281 | ctcgccgggg gccgcccacc gcgacggcaa gtgacccggg ccgggggcgg ggagtcggga |
| 2341 | ggagcggcgg gggcgggcgc cgggggcggg cagaggcgcg ggagagcgcg ccgccggccc |
| 2401 | tttatagcgc gcggggcacc ggctccccaa gactgcgagc tccccgcacc ccctcgcact |
| 2461 | ccctctggcc ggcccagggc gccttcagcc caacctcccc agccccacgg gcgccacgga |
| 2521 | acccgctcga tctcgccgcc aactggtaga catggagacc cctgcctggc cccgggtccc |
| 2581 | gcgccccgag accgccgtcg ctcggacgct cctgctcggc tgggtcttcg cccaggtggc |
| 2641 | cggcgcttca ggtgagtggc accagcccct ggaagcccgg ggcgcgccac acgcaggagg |
| 2701 | gaggcgacag tcctggctgg cagcgggctc gccctggttc cccggggcgc ccatgttgtc |
| 2761 | ccccgcgcct acgggactcg gctgcgctca cccagcccgg cttgaatgaa ccgagtccgt |
| 2821 | cgggcgccgg cgggagttgc agggagggag ttggcgcccc agaccccgct gccccttccg |
| 2881 | ctggagagtt ttgctcgggg tgtccgagta attggactgt tgttgcataa gcggactttt |
| 2941 | agctcccgct ttaactctgg ggaaagggct tcccagtgag ttgcgacctt caatatgata |
| 3001 | ggacttgtgc ctgcgtctgc acgtgttggc gtgcagaggt ttggatatta tctttcatta |
| 3061 | tatgtgcatc ttcccttaat aaagagcgtc cctggtcttt tcctggccat ctttgttcta |
| 3121 | ggtttgggta gaggcaatcc aaaagggctg gattgctgct tagattggag caggtacaac |
| 3181 | gttgtgcatg ccccgtattt ctacgaggtg ttcgggacgg cgtagagact gggacctgct |
| 3241 | gcgtactggc aaagcagacc ttcataagaa ataatcctga tccaatacag ccgacggtgt |
| 3301 | gacaggccac acgtccccgt gggtctctgt ggaagtttca gtgtagcgac atttcagata |
| 3361 | aaagtggaaa aagtgaagtt tggctttttt catttgtatg cagtcctaac tcttgtcaca |
| 3421 | cgtgtgggat ttatcttttt ccataactta ctgaaaaccc ttcctggcgg gctgaacctg |
| 3481 | actcttcctg agctgagtcc tggactggca cactgatggc tctgggctct tcccggtcaa |
| 3541 | gttataacaa ggctttgccc atgaataatt tcaaacgaaa atgtcaagat ccttgccggt |
| 3601 | gtcctgggat tacaaggtga atcttgtcat gaagaaattc taggtctaga aaaaatttga |
| 3661 | agattctttt tctcttgata attcactaat gaagcttttg tggttgaaaa ataaaaagtg |
| 3721 | aggtttatgg tgatgtcagg tgggaaggtg ttttatacat caatacattc gagtgctctg |
| 3781 | aagtgcatgt aataatagct gtttctctgt tgtttaaagg cactacaaat actgtggcag |
| 3841 | catataattt aacttggaaa tcaactaatt tcaagacaat tttggagtgg gaacccaaac |
| 3901 | ccgtcaatca agtctacact gttcaaataa ggtaagctgg gtacagaaaa agaaaattaa |
| 3961 | ggtctttgat gtttctactg tcctatgctg aacaagaatg tctttaaagc tgattactgg |
| 4021 | atgaaattat ttaacagatg acgaagaaga agggattctt ggcaattcgc tggccggtgt |
| 4081 | catactctat taggcctgca acatttccag accttaaact gatagaacat tttaattgtt |
| 4141 | ttaattgttt ttggaaatga tgggagagtt cctaagtgga gtataaactg tggagagatg |
| 4201 | aaccatcttg agtaggcact gaagtgtgct ttgggtcatg atagattaat taatctcatc |
| 4261 | taaacattga tgtctttttc tgttgctgtc tagactgtga acaatgtcta acaccttagg |
| 4321 | gaagaggtgg ggaggaatcc caatgtatac attgccctta agcagtgttt gattcattca |
| 4381 | tctttggact ccatgaatcg aaatctggta gaatacatga tcttagtgga ggaggccaaa |
| 4441 | tgcgtgactc actgagcctg gcagagcaga aatactctgc tgtctgcacc ctctgggtct |
| 4501 | ggtgtggctc tgcttcttgg tgcYtcaact ctgactggca gctgtcccca ggaggcgata |
| 4561 | attcagcatg ttcaatctaa aggttatgac ttccttgatg gttttcacca tattcttggc |
| 4621 | aagtttttgg tttttgaaat gttctaggag gcttggtaga gatcttatga aatagagaat |
| 4681 | agctgctgtg gaaattattt taatgctaat tacataaaag tacaaaagta gcactagcta |
| 4741 | aaacaaaagg tattttgctg ttctgttttg ttttagcttg tgccaggcct tttacagcat |
| 4801 | taggaatgca acttctagat aacgatgcat cttttaagtg aatgttcttg tttttcaaaa |
| 4861 | tgaacttcat gacagtagtt gccaaaccag caaggagaac ttgcatgcat acgtgcatgc |
| 4921 | atgtgtggat atgtatgggg gtggggggag agaaagatga aggaatttca taacatgaaa |
| 4981 | taatgattac agttctggtc aaacttgtca attcagattt caccaattga gaattagtaa |
| 5041 | gtaatttctc tgatacaggc ctgaagttta ccttagtaaa cactttactt ccatatggta |
| 5101 | aaaattagat tttgggagga atgcttacct cctaaatata ttcaatctaa tatttgaggg |
| 5161 | acacatggga atatatttat gattcatctg ctttttaaac ataagccttt gttaactgta |
| 5221 | agttcttgaa ctttataagg ctgctgttat ttaaatgagc acagctcctg atctgcaaac |
| 5281 | agcagagcgc agggctacag cttgggggat gccagccgac tcagggtggt cctatggact |
| 5341 | gaacaatctc ttgctgctgt actggagggc ctgggagctt ttccatcagc ctcggcctga |
| 5401 | ggtgtgcact cttctcctgc ccaccccagg aataaatgag attcctggtt aaaaaggacc |
| 5461 | agagcagtca ttttacagtt gaggaaactg ttgctctgag aagtgaggga tttattcatg |
| 5521 | actacactga tggtgagtgc ccatgtcagg tctggaacca aagtctaccc agtatccaca |
| 5581 | caccaccatc cctcaggtgg ctctgccaca gtctgatggg aggctccaaa gcgggaggaa |
| 5641 | gaaggaaagt cttgcccact gcatctcctc agttggcctt cctctctgcc tgttttccct |
| 5701 | ccctacagtt agcatcttaa gcagctgccc ctcttccctc ccgactgctc tcactactgc |
| 5761 | agcctggctc cagccgcagg acactactgc tgtgcagaag cccctacttg gaactccaac |
| 5821 | tgcatttttc acctttgcta acagttttca gtggtggttg ggaaatgtta ttggcttaag |
| 5881 | ccttagcaca aaccgtcacc ggtgatattc attccatgga aatgttctga attctaaagc |
| 5941 | tgaatttaca aagcttctgg aaaacaacct gcaaccaaat tagtgactga attttttagt |
| 6001 | taactcaaaa ttccaaatca gagggttttg caatgcctgg aggaaccttg gaggctttta |
| 6061 | aagtgttaat gctattaatg gcattcagag ggattttcta cagaattgtc ccttcattac |
| 6121 | ctgtttatac agttttacta cttaccaggg tactgtataa atccttgtgc taaattttgc |
| 6181 | tatagagtat gtggtccctg ctgtgagctg ggaggaacca aatactgtat ctctatgtta |
| 6241 | catagaaagc cctaggagac tttctcctgt tatctgaaca actatttgct gtactgataa |
| 6301 | aaaggaaaca gcatagtctc attcactttt tgaaatggaa atgataaaat aaaacacatt |
| 6361 | ttggtcattc gggaacaaaa taccctctct acttttatca cataaaatta aataaataga |
| 6421 | aaccaaaata tttcagtatc aatcttagtt tgtgcacttt aggataaaga atgtgtttac |
| 6481 | ccaaatcctt ttggcctggt tacttagttc agattttgaa agaaaatata tttgtggctt |
| 6541 | ttatgtgtga atttagacaa tggaatccat gtggtgcctc gttttccctg agattatgta |
| 6601 | ttaattcaac ctgtaaatgc aaaccatcta atagtcagcg agaccctata gccctgctgc |
| 6661 | ttaatggggg cacacaaggg catgcagccc tcgtaccagg cagactgtgt tcatattaac |
| 6721 | agcatcgtgg agaaactcat gctgggggac aggggaggga gatgtaaatg ctcagcaggg |
| 6781 | agatctggag attcctggag caggtggagt tgggacctgg ccttgaacga tgggtctggc |
| 6841 | tctggcagtc agtaatgcca aagggaagag cagcataact gtcactttcc atgggacaga |
| 6901 | agtgtgtgaa tcaagttgca gtgacgcttc acctatttat tattttggtc atttagaaga |
| 6961 | atttcattgt cagtagaagt cctttaaatc atttcccctt cagtgacgtc tcacaaaaga |
| 7021 | aagatctgtc tttagctttt tagtctcaga ctttattaga cagatactac ctgtactctt |
| 7081 | attctgtaat ctttgttggg atggattcac atcttgcaaa ggaagggagg catgtagtat |
| 7141 | aatggggcaa acagacccag ctctgccact cgttagatat gtgaccttct gcaagttgct |
| 7201 | tagtgcctgt gagcttcagt gtcctcatgg ataagaaaga tccaacacct tcttggaagg |
| 7261 | attatatcaa atgaagtaac atgagtaaag ggtccagcag aatacctggc atatagtgga |
| 7321 | gtcaatgaat gattaataat attattaata gtggtcatga gagaatatat gtataacatg |
| 7381 | ttattatgta gactcactat atagactcta ttctacatag aatatagaac attatataac |
| 7441 | aaacaactat aataagtaga ctatagtaaa caacctcact ttgtctcagt tgcctcatct |
| 7501 | tgatggaaaa ctgctctttc tctcctgtta ccttgacaga gagcgtctac attctaaaag |
| 7561 | aaagatattt aacaaaatgg ttgagtacag atccaagagt caaatagctg tctggttcaa |
| 7621 | agtccagctg tgtgattttg agctagtcac ccaatctcac tttgtctcag tagccttatt |
| 7681 | tgtaaaaaca aggcaaatta cagagccatc ccctgggttg ctatgaggac tcaaacatgc |
| 7741 | atcccaagtg ctcggtgttg ctaggtatga tggctcacac ctgtacattc agcactttgg |
| 7801 | gaggccgaag cagaaggatc agcctgggca acatagcagg accccatctc tacaaaacaa |
| 7861 | tgtttaaaaa aaagcaaagt gctcagcaca gtgactgcat cattaggatt gattgtaggg |
| 7921 | ctcctgatgt tagcacagaa caccacagcc aggaagcagt ctatcttgtt gggtgcaaat |
| 7981 | tgtaacattc catttatgtt tcttccttct tttctttctt tagcactaag tcaggagatt |
| 8041 | ggaaaagcaa atgcttttac acaacagaca cagagtgtga cctcaccgac gagattgtga |
| 8101 | aggatgtgaa gcagacgtac ttggcacggg tcttctccta cccggcaggg aatgtggaga |
| 8161 | gcaccggttc tgctggggag cctctgtatg agaactcccc agagttcaca ccttacctgg |
| 8221 | agagtaagtg gcttgggctg taataccgtt cattcttgtt agaaacgtct gaacattctc |
| 8281 | gtgatcttgt gcctttaggg gctacaaaat taaaaatatt tattcttttt ttctcagaaa |
| 8341 | ctggtatgta tcacagccct cttcacacat tccagatgtg gtaggaggtt cacagaatgt |
| 8401 | gaactttgga gctgatgaca gtgtcatcaa gtaactttct cccccagtct gtccccagac |
| 8461 | cctgttactg tcctcagtaa ccggctgaat gtgtgttggg agagggcggg ccagggaagc |
| 8521 | gggtagggat aggaaatcca ccaaggccgg ggttttagct tttccctata tatatatcat |
| 8581 | gtatcctgat ttttctgtcc cgttatcaca ctaaaaatcc cagttgagga tttttcccaa |
| 8641 | acggtcataa atcaatgagg aaagtccatg gtttccctct gagcccataa ttagcctaat |
| 8701 | tatgctgacc ttttctaatc agttggccat gatttgagtt ccgtgatgtg ccagcacctg |
| 8761 | cccagccatc tgcctgtcac cctcgttctg gttttggaaa ggtggaatac tttcctcctc |
| 8821 | agcctttgcc cctgtaagct ggccctagga gccagtaaaa gaatgaagag aattcctgtc |
| 8881 | aagtaggaga tttattcttt tgccgcaact gtggctctga gctaggcaat ttagataaat |
| 8941 | gcatgtagca cattgagtag agtgaaatta gcttctcttg taaggccagc tggttagaat |
| 9001 | gaaggtgttg tgtgagtgtt aggcccagcg agagagaaca gtttctcaag gtaggaatgg |
| 9061 | tgaaaagaag gggtggacgg acaaccaacc aaccatcctc ctctggtatc tactttgagg |
| 9121 | gttgaaatag ggggcctgac cccaggtgaa tgtggctgcc ttcccagagc ccccatttgc |
| 9181 | aagaccctcc agacccccag gtgcttctgc ttgtgtcttt tgtggcacca ggcaagaatg |
| 9241 | tagcagcgtc agcagcccct ctggtgactg tggcatggtt gacattcatt tcccccctaa |
| 9301 | ttaatggcat cctcatgatt ctcttttata ttaatagttc ttgagttttt ttgtaagcta |
| 9361 | cttcaaatcc tttgttggtg caagatagaa gatattttat gtgtttgttt tgcatgtgca |
| 9421 | cacacatatt tggcctgtga attgatgttt gttttcctgt catttaacca aagcacatga |
| 9481 | gataattgag ccattgcaga gaccccgtgg ttaaatccgg cttctcgagg taccaaggac |
| 9541 | atttcctggg ctttctcaca gccctacata tttttgaacc taaaatatcg tagtttatgc |
| 9601 | taccaccctg ttcagtatag tagccactag ccacatgtgg ctgttgacca cttgaaatat |
| 9661 | ggctaatgct ctaagtataa agtacacact ggaatttaag aagtgtagaa tatctcaaaa |
| 9721 | cttttttata ttgattacac attaaaatga ttatattcca gatatatgca gttgactcaa |
| 9781 | gcaatgcatg gctgagaggc accgactccc tgtgcagttg aaaatccgag tataacttga |
| 9841 | ctccccaaaa acttaactac taatagccta cctatcggtt gactgttgac tgcagcctta |
| 9901 | ccaataagat aaacagtcaa ttaacacaca tttttcatgt tgcgtgtatt atatactgta |
| 9961 | ttcttacaat aaagtaagct agaggaaaga aaatgttatt aagaaaatta taaggaaaag |
| 10021 | aggctgggca tggtggctcg tgcctgtaat ctcagaactt tgggatgcta aggcgggtgg |
| 10081 | atcacttgag gtcaggagtt caagaccagc ctggccaaca tggtgaaacc ccatctctac |
| 10141 | taaaaataca aaaattagcc aggcgtggtt gtgggtgcct gtaatcccag ctacttggga |
| 10201 | ggctgaggca ggagaatcac ttcaacccag gtggaggagg ttgcagtgaa ctgagattgc |
| 10261 | gccactgcac tccggcctgg gtgacagagc gagactctgt ctaaaaaaga aagggaaaga |
| 10321 | aagaaaaaaa agaaaagaaa agaaaagaaa gaaggaagga agagaaagaa ttataaggaa |
| 10381 | gagaaaatat atttactatt gataaagtgg aagtggatca tcataaaggt gttcatcctc |
| 10441 | gtcatcttca tgttgagtag gctgaggagg aggaggagga ggaagagcag gggccacggc |
| 10501 | aggagaaaag atggaggaag taggaggcgg cacacttggt gtaactttta tttaaaaaaa |
| 10561 | tttgcataca agtggatcca cagagttcaa acccatgttg ttcaggggtc aactgtcttt |
| 10621 | ggttaaataa aatatattat taaaattaat ttcacctgtt cctttttact ttttctaatg |
| 10681 | tgactactag aaaacttaaa atgacatctg aggctccatt gtcttcccct tgggccagca |
| 10741 | ctaccacaga atgtcttagg attcagctcc aggccgccac gcctgcttct ttcagggagc |
| 10801 | tggttctatg cacatgtttt atatgagaga taattaagtt gtcaattgtg ataacaaaac |
| 10861 | aggatttgac tttgtacaga attctttggt tccaaccaag ctcatttcct ttgtttcagc |
| 10921 | aaacctcgga cagccaacaa ttcagagttt tgaacaggtg ggaacaaaag tgaatgtgac |
| 10981 | cgtagaagat gaacggactt tagtcagaag gaacaacact ttcctaagcc tccgggatgt |
| 11041 | ttttggcaag gacttaattt atacacttta ttattggaaa tcttcaagtt caggaaaggt |
| 11101 | gagcattttt taatttgttt ttatgacctg ttttaaattg tgaatacttg ggttttacaa |
| 11161 | cccatttctt ccccaattca aaaatagcag aacagagttg ttgagaaggt gatggagtag |
| 11221 | aagggggagc gcgcactgtg gggaggggtg gacaacaggc ctggtcctac ctgtgactct |
| 11281 | gcactaccct gtgactctgg gcagggcccc ctcggagacc caggttcctc agccaaccgg |
| 11341 | ctggatcagg tcatctctaa aggtcccgcc acgctcacat ttctccctct attgaggatc |
| 11401 | ccaggcacaa aatttgtttt tggttcaatg cataatactc ccttcctttt tcttttactg |
| 11461 | cagatatctt ctaaaggggc tcaatagggt tcaatatgcc taaattggat cttctcagtc |
| 11521 | ttggaaaagg catttttagc agtgatcaag ggaaactgat tagcgaagtc acttctaatc |
| 11581 | cttcacgtgt cagctgtgtt cttgtaggct ttgcttagaa cctaggtttt tacttccaca |
| 11641 | gtgacttaat aaaggggaaa gaattgactc agagcccaga tgaattaaga actctatctt |
| 11701 | tttacagaaa acagccaaaa caaacactaa tgagtttttg attgatgtgg ataaaggaga |
| 11761 | aaactactgt ttcagtgttc aagcagtgat tccctcccga acagttaacc ggaagagtac |
| 11821 | agacagcccg gtagagtgta tgggccagga gaaaggggaa ttcagaggtg agtggctctg |
| 11881 | ccagccattt gcctgggggt atgggtgctg tgggtgactt ctggaggagt agctccaccc |
| 11941 | tcagggctgg gatatacttc cttggttaaa tattcaggaa aacaaactgc cctggaggtt |
| 12001 | ttttgttgtt atttgtttgt tttggttttg attttgcttt ggtacaaaaa agattttgga |
| 12061 | catttagaaa tgtttctgtg ttgattgtgc ccttgtatta gcaggtgttt tcttgagcac |
| 12121 | ctgtcatgtg ctaagccctc tgctgagcac tggatacaca aactgtgttt aggatttagc |
| 12181 | aacaagtcac agatttccct gggcattttt tcatgcttaa attctaattc tgggggtggc |
| 12241 | ttctggacca gctgcagcag gacacagtag acattcgtga gtacccactg tgggctgttg |
| 12301 | ccacagaggc tgtagagtct aacccatcaa gggaagggat tgagtatatc aaatataccc |
| 12361 | acatgcatgc atgtgtgtat. atggcggaca cgtgtgtgta catgcatgtg catatgttgg |
| 12421 | gagctcaggc ccattgtgcg aggaacagtc cctaaccgga agtgctgtgg gccttcagac |
| 12481 | tcttgcagga agctgcaagc ctgtgtgtct cgatccatgc cttacaggga aagtattctg |
| 12541 | agtactttca gtgaagaaaa gagtcagggg atataaacga tggcttacgc tgggtgtggt |
| 12601 | ggctcacgcc tgtagtccct gcactttggg aggcccagac aggcaaatca cttgaggtca |
| 12661 | ggagtttggg accagcctgg ccaacatggt aaaagcccat ctctactcaa aatacaaaaa |
| 12721 | gtagctgggt gtggttgcac gtgtctgtag tcccagctac tcaggaggtt gaggcaggag |
| 12781 | aattgcttga acctgggagg cggaggctga agtgagctga gattggacca ctgtactcca |
| 12841 | gcctgggtga cagagcgaga ttccatctca aaaaaaaaaa aaaagaaaca acgaaaaaag |
| 12901 | aaatgatggc ttagctccat gtgaagatga tatttgaaca ttttaaaaca ctttaaataa |
| 12961 | actgttctct cctgtttatt gccactgaca ggagaggttt ctctttacct ctggtcctgc |
| 13021 | acccctctga gccatcctac ccacagcctt cagtcattgt cctaaagcct agctctaatt |
| 13081 | ccactgcctc tccttttgtg cacacacact tctctgcttc cctggccgtt ctctatcttg |
| 13141 | gagaggcatt tcaaacgcca cttccaccag aaggccttgc tactgcacca actagttact |
| 13201 | atctcttctt cacccaaatc ctggtagcac tttggatctc ccactttgca cttagggttc |
| 13261 | accttccgtt ataatcattg ccatcaatct cagcatcgtt tttaggcact tctttccagc |
| 13321 | cattgttctt acctccaact acatatcttt tctggactgt gcattattca gtttattaaa |
| 13381 | tgcccattaa atgtgtttag ccattgtcaa ttactctgaa acgttcaggt tttgacaaat |
| 13441 | tctttcctaa tgtaagtgtg gtggaaagag tgaaagaaag tcaaattgca caaaaatagg |
| 13501 | atggtgtaat ttggggttat gccgtcaatt ttgtccactg ataaatggga tttgagctct |
| 13561 | ccaagttgac tagatgccct ttatttttca gaaatattct acatcattgg agctgtggta |
| 13621 | tttgtggtca tcatccttgt catcatcctg gctatatctc tacacaagtg tagaaaggca |
| 13681 | ggagtggggc agagctggaa ggagaactcc ccactgaatg tttcataaag gaagcactgt |
| 13741 | tggagctact gcaaatgcta tattgcactg tgaccgagaa cttttaagag gatagaatac |
| 13801 | atggaaacgc aaatgagtat ttcggagcat gaagaccctg gagttcaaaa aactcttgat |
| 13861 | atgacctgtt attaccatta gcattctggt tttgacatca gcattagtca ctttgaaatg |
| 13921 | taacRaatgg tactacaacc aattccaagt tttaattttt aacaccatgg caccttttgc |
| 13981 | acataacatg ctttagatta tatattccgc actcaaggag taaccaggtc gtccaagcaa |
| 14041 | aaacaaatgg gaaaatgtct taaaaaatcc tgggtggact tttgaaaagc tttttttttt |
| 14101 | tttttttttt tgagacggag tcttgctctg ttgcccaggc tggagtgcag tagcacgatc |
| 14161 | tcggctcact gcaccctccg tctctcgggt tcaagcaatt gtctgcctca gcctcccgag |
| 14221 | tagctgggat tacaggtgcg cactaccaca ccaagctaat ttttgtattt tttagtagag |
| 14281 | atggggtttc accatcttgg ccaggctggt cttgaattcc tgacctcagg tgatccaccc |
| 14341 | accttggcct cccaaagtgc tagtattatg ggcgtgaacc accatgccca gccgaaaagc |
| 14401 | ttttgagggg ctgacttcaa tccatgtagg aaagtaaaat ggaaggaaat tgggtgcatt |
| 14461 | tctaggactt ttctaacata tgtctataat atagtgttta ggttcttttt tttttcagga |
| 14521 | atacatttgg aaattcaaaa caattggcaa actttgtatt aatgtgttaa gtgcaggaga |
| 14581 | cattggtatt ctgggcacct tcctaatatg ctttacaatc tgcactttaa ctgacttaag |
| 14641 | tggcattaaa catttgagag ctaactatat ttttataaga ctactataca aactacagag |
| 14701 | tttatgattt aaggtactta aagcttctat ggttgacatt gtatatataa ttttttaaaa |
| 14761 | aggttttcta tatggggatt ttctatttat gtaggtaata ttgttctatt tgtatatatt |
| 14821 | gagataattt atttaatata ctttaaataa aggtgactgg gaattgttac tgttgtactt |
| 14881 | attctatctt ccatttatta tttatgtaca atttggtgtt tgtattagct ctactacagt |
| 14941 | aaatgactgt aaaattgtca gtggcttaca acaacgtatc tttttcgctt ataatacatt |
| 15001 | ttggtgactg taggctgact gcacttcttc tcaatgtttt ctcattctag gatgcaaacc |
| 15061 | aatggagaag cccctaatta gatcagggca gagggaaaaa caaaaaactg gtagaaaccg |
| 15121 | gcaaccacag cttcaagctt taagcccatc tcctacactt ctgctctgta cgtgcccatt |
| 15181 | gtcacttctg ttcacatgct actgtcccaa gcaagtgacc aagcctgaca atactttgtc |
| 15241 | tactggagtc actgcaaggc acatgacggg gcagggatgt cgtcttacag ggaagagaaa |
| 15301 | agataatgct ctctactgca gacttggaga gatttcttcc cattggcagt agtttgacta |
| 15361 | attggagatg agaaaaaaag aaacattctt gggatgattg tattgaaaca aaattaggta |
| 15421 | aaaggacaat ataggatagg gagagatata agtggaatga gatctctaga gtccattaaa |
| 15481 | agcaagctag attgagagct cttggagggc agggactggg cctagctcat ggtttacagc |
| 15541 | tcttgagggt gactgcacag tggactcagt cagtcatggc tgagttgagt tggctttatt |
| 15601 | atctctagaa tgtagttctc cattcaaatg caaaacagcc ttattctctc aactgtagta |
| 15661 | aaagaggatt ctaactagag atgaaaaagg ttcttggcac ctactgggcc acaagactgc |
| 15721 | ttttgcttcc cactgtggaa gggggcactg ttatctagga aaaacatcca gatggcacac |
| 15781 | ctactcagaa gtggttccac aaagaaattg agcaagcatt cagatgagca gcctcagctc |
| 15841 | actatctagc tgttaataaa ttttaagtcc gttttgttcc tttgcaccaa agaaattgaa |
| 15901 | caatgtcaat aaaattccaa acgtgaactg ttttatgtaa caccacatcc taaggaagaa |
| 15961 | tgcctgaaac aaacctttga tgtcctcata aaaatgatag ccattttgga gaaattatgg |
| 16021 | aaaacagacc agtggttggt ttatttggtt ggttttgttt attattactt ttttttggcc |
| 16081 | atcaacacag aggctagaga ggttgaatct tgcactgttg tagtgttttg ttatacagag |
| 16141 | aaaagggatg tatttgcagc agagtcaaca aaaaagaaac tggcatgggg gcacatcctc |
| 16201 | aaggtgcttc aacctttatc tcaaattcct gctcacatgg ggctacctgg gacaaagagc |
| 16261 | ttagtgcatt acatgaggag gcagaacaag ctacagcaga aggtgcctaa ccagatattt |
| 16321 | ccctgccagg attaattata gcattatgta atttcttggc ctcattatct aaggaatttg |
| 16381 | aattattcca ctaaggaaaa tactggcaga tagaaaacag gtctgggtac taatgaggaa |
| 16441 | tctggctgat cacaaggaag ggattcatta tggactggaa tgaggctgaa ctgcaataat |
| 16501 | cagaaagctt ccacacagca aatgttaacc actccctact ttgccacatc cctctgaatt |
| 16561 | gctgagacct aacagccgat actaaaaatc agcactgtga ttgacactgg ggcagaaacc |
| 16621 | ataccaaaga ctgttggctg caatccagaa actgttgccc tccagtggac caaattatac |
| 16681 | ttcattactt gaccagtaat gtaccccata gagctaattg ttagttaacc aaactatcat |
| 16741 | gcattctttg ttttgttttg tgagacagag tctggctttg tctccaggct ggagtgcagt |
| 16801 | ggtgagatct cggctcactg caacctctgc ctcccaggtt caagcgattc tcgtgcctca |
| 16861 | gccttctgag tagctgggat tacaggtgtg agccaccatg cctggctaat ttttgtattt |
| 16921 | ttagtagaga tagggttttg ccatgttggc aaactcctga cttcaagtga tccacccacc |
| 16981 | tcggcctccc aaagtgctga gattataggc gtgagccacc atgcccagcc ctttcatgcg |
| 17041 | ttctaaagat atttttccaa tcttaaatta ttaactgaat ttggcttaca tataagaaac |
| 17101 | taatactcat gaaatccaaa gacattttag cttaatttca gctgatggct tataatctaa |
| 17161 | ggaactgccc cttaaacaat tatctctatt catcaaatgg tgaataaact cgttcccaaa |
| 17221 | tg |

An “allele” is defined as any one or more alternative forms of a given gene at a particular locus on a chromosome. Different alleles produce variation in inherited characteristics such as hair color or blood type. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene. In an individual, one form of the allele (major) may be expressed more than another form (minor). When “genes” are considered simply as segments of a nucleotide sequence, allele refers to each of the possible alternative nucleotides at a specific position in the sequence. For example, a CT polymorphism such as ^{5′}CCT[C/T]CCAT^{3′} would have two alleles: C and T (also represented by Y). Furthermore, depending on the strand that is represented (i.e. ^{5′}ATGG[G/A]AGG^{3′}) the SNP may be a Y or an R in this example (see also SEQ ID NOs:17, 5 and 1).

A “gene” is an ordered sequence of nucleotides located in a particular position on a particular chromosome that encodes a specific functional product and may include untranslated and untranscribed sequences in proximity to the coding regions. Such non-coding sequences may contain regulatory sequences needed for transcription and translation of the sequence or introns etc.

A “genotype” is defined as the genetic constitution of an organism, usually in respect to one gene or a few genes or a region of a gene relevant to a particular context (for example the genetic loci responsible for a particular phenotype). A region of a gene can be as small as a single nucleotide in the case of a single nucleotide polymorphism.

A “phenotype” is defined as the observable characters of an organism.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins”) of a nucleotide relative to a reference allele. Furthermore, it would be appreciated by a person of skill in the art, that an insertion or deletion within a given sequence could alter the relative position and therefore the position number of another polymorphism within the sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute, and D) white blood cell count >12,000 per mm3 or <4,000 mm3. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Patient outcome or prognosis as used herein refers the ability of a patient to recover from an inflammatory condition. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome or prognosis may be accomplished by various methods. For Example, an “APACHE II” score is defined as Acute Physiology And Chronic Health Evaluation and herein was calculated on a daily basis from raw clinical and laboratory variables. Vincent et al. (Vincent J L. Ferreira F. Moreno R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.” Furthermore, the criteria or indication for administering activated protein C (XIGRIS™-drotrecogin alfa (activated)) in the United States is an APACHE II score of ≧25. In Europe, the criteria or indication for administering activated protein C is an APACHE II score of ≧25 or 2 organ to system failures.

“Activated protein C” is also known as Drotrecogin alfa (activated) and is sold as XIGRIS™ by Eli Lilly and Company. Drotrecogin alfa (activated) is a serine protease glycoprotein of approximately 55 kilodalton molecular weight and having the same amino acid sequence as human plasma-derived Activated Protein C. The protein consists of a heavy chain and a light chain linked by a disulfide bond. XIGRIS™, Drotecogin alfa (activated) is indicated for the reduction of mortality in adult subjects with severe sepsis (sepsis associated with acute organ dysfunction) who have a high risk of death (e.g., as determined by an APACHE II score of greater >25 or having 2 or more organ system failures).

XIGRIS™ is available in 5 mg and 20 mg single-use vials containing sterile, preservative-free, lyophilized drug. The vials contain 5.3 mg and 20.8 mg of drotrecogin alfa (activated), respectively. The 5 and 20 mg vials of XIGRIS™ also contain 40.3 and 158.1 mg of sodium chloride, 10.9 and 42.9 mg of sodium citrate, and 31.8 and 124.9 mg of sucrose, respectively. XIGRIS™ is recommended for intravenous administration at an infusion rate of 24 mcg/kg/hr for a total duration of infusion of 96 hours. Dose adjustment based on clinical or laboratory parameters is not recommended. If the infusion is interrupted, it is recommended that when restarted the infusion rate should be 24 mcg/kg/hr. Dose escalation or bolus doses of drotrecogin alfa are not recommended. XIGRIS™ may be reconstituted with Sterile Water for Injection and further diluted with sterile normal saline injection. These solutions must be handled so as to minimize agitation of the solution (Product information. XIGRIS™, Drotecogin alfa (activated), Eli Lilly and Company, November 2001).

Drotrecogin alfa (activated) is a recombinant form of human Activated Protein C, which may be produced using a human cell line expressing the complementary DNA for the inactive human Protein C zymogen, whereby the cells secrete protein into the fermentation medium. The protein may be enzymatically activated by cleavage with thrombin and subsequently purified. Methods, DNA compounds and vectors for producing recombinant activated human protein C are described in U.S. Pat. Nos. 4,775,624; 4,992,373; 5,196,322; 5,270,040; 5,270,178; 5,550,036; 5,618,714.

Treatment of an inflammatory condition using activated protein C or protein C like compound in combination with a bactericidal and endotoxin neutralizing agent is described in U.S. Pat. No. 6,436,397; methods for processing protein C is described in U.S. Pat. No. 6,162,629; protein C derivatives are described in U.S. Pat. Nos. 5,453,373 and 6,630,138; glycosylation mutants are described in U.S. Pat. No. 5,460,953; and Protein C formulations are described in U.S. Pat. Nos. 6,630,137, 6,436,397, 6,395,270 and 6,159,468,

Alternatively, the treatment of an inflammatory condition may also be achieved through the use of an inhibitor to the tissue factor pathway including but not limited to antibodies, inhibitors, and antagonists to coagulation factor III (Tissue Factor), FACTOR VII and FACTOR X. For example, US20030207895 describes pharmaceutically active compounds which are tissue factor (coagulation factor III) antagonists. Antagonists may include an anti-tissue factor monoclonal antibodies such as TNX-832 currently in development for acute lung injury (ALI) and acute respiratory distress syndrome (ARDS), which is in a Phase 1/2 clinical trial for the treatment of ALI/ARDS. Similarly, other tissue factor pathway antagonists are known, such as the serine protease inhibitors described in US2003212071. In addition, pharmaceutical compositions having a tissue factor antagonist properties are known (for example, WO2004041296 and WO2004041302).

A “Brussels score” score is a method for evaluating organ dysfunction as compared to a baseline. If the Brussels score is 0 (i.e. moderate, severe, or extreme), then organ failure was recorded as present on that particular day (see TABLE 1F below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR)>1.5 are days that a person is alive and does not have an INR>1.5.

TABLE 1F
Brussels Organ Dysfunction Scoring System

| | ORGANS | | | | |
|---|---|---|---|---|---|
| | Free of | | | | |
| | Organ Dysfunction | | Clinically Significant Organ Dysfunction | | |
| | Normal | Mild | Moderate | Severe | Extreme |
| | DAF ORGAN DYSFUNCTION | | | | |
| | SCORE | | | | |
| | 1 | | 0 | | |
| Cardiovascular | >90 | ≦90 | ≦90 | ≦90 plus | ≦90 |
| Systolic BP | | Responsive to | Unresponsive to | pH ≦ 7.3 | plus |
| (mmHg) | | fluid | fluid | | pH ≦ 7.2 |
| Pulmonary | >400 | 400-301 | 300-201 | 200-101 | ≦100 |
| Pₐo₂/F_{I}o₂ | | | Acute lung | ARDS | Severe ARDS |
| (mmHg) | | | injury | | |
| Renal | <1.5 | 1.5-1.9 | 2.0-3.4 | 3.5-4.9 | ≧5.0 |
| Creatinine | | | | | |
| (mg/dL) | | | | | |
| Hepatic | <1.2 | 1.2-1.9 | 2.0-5.9 | 6.0-11.9 | ≧12 |
| Bilirubin | | | | | |
| (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

Analysis of variance (ANOVA) is a standard statistical approach to test for statistically significant differences between sets of measurements.

The Fisher exact test is a standard statistical approach to test for statistically significant differences between rates and proportions of characteristics measured in different groups.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with dyslipidemia subjects with chronic renal failure, subjects with bronchiectasis, subjects with chronic obstructive lung disease, chronic bronchitis, emphysema, or asthma, subjects with hypertension or pulmonary hypertension, subjects with cardiovascular disease, subjects with acute coronary syndrome subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism, venous thrombosis, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by Chomczynski (U.S. Pat. No. 5,945,515), whereby genetic material can be extracted efficiently in as little as twenty minutes. Evans and Hugh (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the F3 sequence, provided that the genetic material obtained contains the sequence of interest. Particularly, a person may be interested in determining the F3 genotype of a subject of interest, where the genotype includes a nucleotide corresponding to position 599, 1089, 1826, 4524 or 13925 of SEQ ID NO:1-5. The sequence of interest may also include other F3 polymorphisms or may also contain some of the sequence surrounding the polymorphism of interest. Detection or determination of a nucleotide identity or the genotype of one or more single nucleotide polymorphism(s) (SNP typing), may be accomplished by any one of a number methods or assays known in the art. Many DNA typing methodologies are useful for allelic discrimination and detection of SNPs. Furthermore, the products of allelic discrimination reactions or assays may be detected by one or more detection methods. The majority of SNP genotyping reactions or assays can be assigned to one of four broad groups (allele specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, allele specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and allelic discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, allele specific extensions, or simple PCR amplification) are useful in allelic discrimination reactions. For example, in mini sequencing a primer anneals to its target DNA immediately upstream of the SNP and is extended with a single nucleotide complementary to the polymorphic site. Where the nucleotide is not complementary no extension occurs.

Oligonucleotide ligation assays generally have two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an invader probe and allele specific probes to anneal to the target DNA with an overlap of one nucleotide. When the allele specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the major allele and the other to the minor allele. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragment. Mismatched probes are displaced without fragmentation. The genotype of a sample is determined by measuring the signal intensity of the two different dyes.

It will be appreciated that numerous other methods for allelic discrimination and detection are known in the art and some of which are described in further detail below. It will also be appreciated that reactions such as arrayed primer extension mini sequencing, tag microarrays and allelic specific extension could be performed on a microarray. One such array based genotyping platform is the microsphere based tag-it high throughput genotyping array (Bortolin S. et al. Clinical Chemistry (2004) 50(11): 2028-36). This method amplifies genomic DNA by PCR followed by allele specific primer extension with universally tagged genotyping primers. The products are then sorted on a Tag-It array and detected using the Luminex xMAP system.

SNP typing methods may include but are not limited to the following:

Restriction Fragment Length Polymorphism (RFLP) strategy—An RFLP gel-based analysis can be used to distinguish between alleles at polymorphic sites within a gene. Briefly, a short segment of DNA (typically several hundred base pairs) is amplified by PCR. Where possible, a specific restriction endonuclease is chosen that cuts the short DNA segment when one variant allele is present but does not cut the short DNA segment when the other allele variant is present. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the initial DNA sample had the allele, which could be cut by the chosen restriction endonuclease. In contrast, if only one higher molecular weight band is observed (at the molecular weight of the PCR product) then the initial DNA sample had the allele variant that could not be cut by the chosen restriction endonuclease. Finally, if both the higher molecular weight band and the two lower molecular weight bands are visible then the initial DNA sample contained both alleles, and therefore the subject was heterozygous for this single nucleotide polymorphism;

Sequencing—For example the Maxam-Gilbert technique for sequencing (Maxam A M. and Gilbert W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by Sanger et al. (Sanger et al. Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxy-nucleotides have been used to sequence encephalomyocarditis virus RNA (Zimmern D. and Kaesberg P. Proc. Natl. Acad. Sci. USA (1978) 75(9):4257-4261). Mills D R. and Kramer F R. (Proc. Natl. Acad. Sci. USA (1979) 76(5):2232-2235) describe the use of Qu replicase and the nucleotide analog inosine for sequencing RNA in a chain-termination mechanism. Direct chemical methods for sequencing RNA are also known (Peattie D A. Proc. Natl. Acad. Sci. USA (1979) 76(4):1760-1764). Other methods include those of Donis-Keller et al. (1977, Nucl. Acids Res. 4:2527-2538), Simoncsits A. et al. (Nature (1977) 269(5631):833-836), Axelrod V D. et al. (Nucl. Acids Res. (1978) 5(10):3549-3563), and Kramer F R. and Mills D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) is described for large scale screening;

Oligonucleotide ligation assay (OLA)—is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2): 123-9; IANNONE M A. et al. Cytometry (2000) 39(2): 131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8);

Polymerase proofreading methods are used to determine SNPs identities, as described in WO 0181631;

Detection of single base pair DNA mutations by enzyme-amplified electronic transduction is described in PATOLSKY F et al. Nat. Biotech. (2001) 19(3):253-257;

Gene chip technologies are also known for single nucleotide polymorphism discrimination whereby numerous polymorphisms may be tested for simultaneously on a single array (EP 1120646 and Gilles P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (Haff L A. and Smirnov I P. Nucleic Acids Res. (1997) 25(18):3749-50; Haff L A. and Smirnov I P. Genome Res. (1997) 7:378-388; Sun X. et al. Nucleic Acids Res. (2000) 28 e68; Braun A. et al. Clin. Chem. (1997) 43:1151-1158; Little D P. et al. Eur. J. Clin. Chem. Clin. Biochem. (1997) 35:545-548; Fei Z. et al. Nucleic Acids Res. (2000) 26:2827-2828; and Blondal T. et al. Nucleic Acids Res. (2003) 31(24):e155); or

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Alternatively, if a subject's sequence data is already known, then obtaining may involve retrieval of the subject's nucleic acid sequence data from a database, followed by determining or detecting the identity of a nucleic acid or genotype at a polymorphism site by reading the subject's nucleic acid sequence at the polymorphic site.

Once the identity of a polymorphism(s) is determined or detected an indication may be obtained as to subject outcome or prognosis or ability of a subject recover from an inflammatory condition based on the genotype (the nucleotide at the position) of the polymorphism of interest. In the present invention, polymorphisms in coagulation factor III (F3) sequence, are used to obtain a prognosis or to make a determination regarding ability of the subject to recover from the inflammatory condition. Methods for determining a subject's prognosis or for subject screening may be useful to determine the ability of a subject to recover from an inflammatory condition. Alternatively, single polymorphism sites or combined polymorphism sites may be used as an indication of a subject's ability to recover from an inflammatory condition, if they are linked to a polymorphism determined to be indicative of a subject's ability to recover from an inflammatory condition. The method may further comprise comparing the genotype determined for a polymorphism with known genotypes, which are indicative of a prognosis for recovery from the same inflammatory condition as for the subject or another inflammatory condition. Accordingly, a decision regarding the subject's ability to recover may be from an inflammatory condition may be made based on the genotype determined for the polymorphism site.

Once subject outcome or a prognosis is determined, such information may be of interest to physicians and surgeons to assist in deciding between potential treatment options, to help determine the degree to which subjects are monitored and the frequency with which such monitoring occurs. Ultimately, treatment decisions may be made in response to factors, both specific to the subject and based on the experience of the physician or surgeon responsible for a subject's care.

Methods of treatment of an inflammatory condition in a subject having an improved response polymorphism in a F3 sequence are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more single nucleotide polymorphism sites in F3 sequence. Also, as previously described the sequence identity of one or more single nucleotide polymorphisms in F3 sequence of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

Once subject outcome or a prognosis is determined, such information may be of interest to physicians and surgeons to assist in deciding between potential treatment options, to help determine the degree to which subjects are monitored and the frequency with which such monitoring occurs. Ultimately, treatment decisions may be made in response to factors, both specific to the subject and based on the experience of the physician or surgeon responsible for a subject's care. Treatment options that a physician or surgeon may consider in treating a subject with an inflammatory condition may include, but are not limited to one or more of the following:
(a) use of anti-inflammatory therapy;
(b) use of steroids;
(c) use of activated Protein C (drotrocogin alpha or XIGRIS™ from Lilly) or protein C like compound;
(d) use of modulators of the coagulation cascade (such as various versions of heparin) use of antibody to tissue factor;
(e) use of anti-thrombin or anti-thrombin III;
(f) streptokinase;
(g) use of antiplatelet agents such as clopidegrel; and
(h) Surfactant.

Alternative treatments currently in development and potentially useful in the treatment of an inflammatory condition may include, but are not limited to the following: antibodies to tumor necrosis factor (TNF) or even antibody to endotoxin (i.e. lipopolysaccharide, LPS); tumor necrosis factor receptor (TNF); tissue factor pathway inhibitors (Tifacogin™ alpha from Chiron); platelet activating factor hydrolase (PAFase™ from ICOS); antibodies to IL-6; antibodies, antagonists or inhibitors to high mobility group box 1 (HMGB-1 or HMG-1 tissue plasminogen activator; bradykinin antagonists; antibody to CD-14; interleukin-10; Recombinant soluble tumor necrosis factor receptor-immunoglobulin G1 (Roche); Procysteine; Elastase Inhibitor; and human recombinant interleukin 1 receptor antagonist (IL-1 RA).

Methods of treatment of an inflammatory condition in a subject having one or more of the risk F3 genotypes associated with improved response to a therapeutic agent are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

### Clinical Phenotype

The primary outcome variable was survival to hospital discharge. Secondary outcome variables were days alive and free of cardiovascular, respiratory, renal, hepatic, hematologic, and neurologic organ system failure as well as days alive and free of SIRS (Systemic Inflammatory Response Syndrome), occurrence of sepsis, and occurrence of septic shock. SIRS was considered present when subjects met at least two of four SIRS criteria. The SIRS criteria were 1) fever (>38° C.) or hypothermia (<35.5° C.), 2) tachycardia (>100 beats/min in the absence of beta blockers, 3) tachypnea (>20 breaths/min) or need for mechanical ventilation, and 4) leukocytosis (total leukocyte count >11,000/μL) (Anonymous. Critical Care Medicine (1992) 20(6):864-74). Subjects were included in this cohort on the calendar day on which the SIRS criteria were met. A subject's baseline demographics that were recorded included age, gender, whether medical or surgical diagnosis for admission (according to APACHE III diagnostic codes (KNAUS W A et al. Chest (1991) 100(6):1619-36)), and admission APACHE II score.

The following additional data were recorded for each 24 hour period (8 am to 8 am) for 28 days to evaluate organ dysfunction, SIRS, sepsis, and septic shock. Clinically significant organ dysfunction for each organ system was defined as present during a 24 hour period if there was evidence of at least moderate organ dysfunction using the Brussels criteria (TABLE 1F) (RUSSELL J A et al. Critical Care Medicine (2000) 28(10):3405-11). Because data were not always available during each 24 hour period for each organ dysfunction variable, we used the “carry forward” assumption as defined previously (Anonymous. New England Journal of Medicine (2000) 342(18): 1301-8). Briefly, for any 24 hour period in which there was no measurement of a variable, we carried forward the “present” or “absent” criteria from the previous 24 hour period. If any variable was never measured, it was assumed to be normal.

To further evaluate cardiovascular, respiratory, and renal function we also recorded, during each 24-hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis). In addition, severity of respiratory dysfunction was assessed, by measuring the occurrence of acute lung injury at the time of meeting the inclusion criteria. Acute lung injury was defined as having a PaO₂/FiO₂ ratio<300, diffuse infiltrates pattern on chest radiograph, and a CVP<18 mm Hg.

To assess duration of organ dysfunction and to correct organ dysfunction scoring for deaths in the 28-day observation period, calculations were made of days alive and free of organ dysfunction (DAF) as previously reported (BERNARD G R et al. New England Journal of Medicine (1997) 336(13):912-8). Briefly, during each 24-hour period for each variable, DAF was scored as 1 if the subject was alive and free of organ dysfunction (normal or mild organ dysfunction, TABLE 1F). DAF was scored as 0 if the subject had organ dysfunction (moderate, severe, or extreme) or was not alive during that 24-hour period. Each of the 28 days after ICU admission was scored in each subject in this fashion. Thus, the lowest score possible for each variable was zero and the highest score possible was 28. A low score is indicative of more organ dysfunction as there would be fewer days alive and free of organ dysfunction.

Similarly, days alive and free of SIRS (DAF SIRS) were calculated. Each of the four SIRS criteria were recorded as present or absent during each 24 hour period. Presence of SIRS during each 24 hour period was defined by having at least 2 of the 4 SIRS criteria. Sepsis was defined as present during a 24 hour period by having at least two of four SIRS criteria and having a known or suspected infection during the 24 hour period (Anonymous. Critical Care Medicine (1992) 20(6):864-74). Cultures that were judged to be positive due to contamination or colonization were excluded. Septic shock was defined as presence of sepsis plus presence of hypotension (systolic blood pressure <90 mmHg or need for vasopressor agents) during the same 24 hour period.

### Microbiology

Microbiological cultures were taken for any subjects who were suspected of having an infection. As this is a cohort of critically ill subjects with SIRS, most subjects had cultures taken. Positive cultures that were suspected of having been contaminated or colonized were excluded. Positive cultures that were deemed to clinically be clinically irrelevant were also excluded. Cultures were categorized as gram positive, gram negative, fungal or other. The sources of the cultures were respiratory, gastrointestinal, skin, soft tissues or wounds, genitourinary, or endovascular.

Haplotypes and Selection of htSNPs

Using unphased Caucasian genotypic data (from the Coriell registry pga.mbt.washington.edu (RIEDER M J et al. SeattleSNPs. NHLBI Program for Genomic Applications, UW-FHCRC, Seattle, Wash. (2001)), haplotypes were inferred using PHASE (STEPHENS M. et al. Am J Hum Genet (2001) 68:978-89) software. MEGA 2 (KUMAR S. et al. (2001) 17:1244-5) was then used to infer a phylogenetic tree to identify major haplotype clades for F3. Haplotypes were sorted according to the phylogenetic tree analysis and the subsequent haplotype structure was inspected to choose haplotype tag SNPs (htSNPs) (JOHNSON G C. et al. Nat Genet (2001) 29:233-7; and GABRIEL S B. et al. Science (2002) 296:2225-9). Six htSNPs marked the major haplotype clades of the coagulation factor III gene (C599T, A1089G, A1826G, C4524T, C12457T, C13925T) and were genotyped in our subject cohorts to define haplotypes and haplotype clades. “Tag” SNPs (tSNPs) or “haplotype tag” SNPs (htSNPs) can be selected to uniquely define a clade and serve as markers for all SNPs within haplotypes of the clade.

### Blood Collection/Processing Genotyping

The buffy coat was extracted from whole blood and samples transferred into 1.5 ml cryotubes and stored at −80° C. DNA was extracted from the buffy coat of peripheral blood samples using a QIAamp DNA Blood Midi Kit (Qiagen™). The genotypic analysis was performed in a blinded fashion, without clinical information. Polymorphisms were genotyped using a real time polymerase chain reaction (PCR) using specific fluorescence-labeled hybridization probes in the ABI Prism 7900 HT Sequence Detection System (Applied Biosystems, Inc.—Livak K J. (1999) Genet Anal 14:143-9). Briefly, the ABI Prism 7900HT uses a 5′ Nuclease Assay in which an allele-specific probe labeled with a fluorogenic reporter dye and a fluorogenic quencher is included in the PCR reaction. The probe is cleaved by the 5′ nuclease activity of Taq DNA polymerase if the probe target is being amplified, freeing the reporter dye and causing an increase in specific fluorescence intensity. Mismatched probes are not cleaved efficiently and thus do not contribute appreciably to the final fluorescent signal. An increase in a specific dye fluorescence indicates homozygosity for the dye-specific allele. An increase in both signals indicated heterozygosity. DNA from lymphocyte cell lines obtained from the Coriell Cell Repository was used to ensure the accuracy of the genotyping. The genotype of these cell lines at 599, 1089, 1826, 4524 and 13925 was determined using the ABI Prism 7900HT Sequence Detection system and compared to the genotype of the same cell lines determined by direct sequencing, given at www.pga.mbt.washington.edu. SeattleSNPs posting for Coagulation factor III occurred on Aug. 22, 2002. (Coagulation factor III. SeattleSNPs. NHLBI HL66682 Program for Genomic Applications, UW-FHCRC, Seattle, Wash. [Online—URL: http://pga.gs.washington.edu).

### Data Collection and Statistical Analysis

Data was recorded for 28 days or until hospital discharge. Raw clinical and laboratory variables were recorded using the worst or most abnormal variable for each 24 hour period with the exception of Glasgow Coma Score, where the best possible score for each 24 hour period was recorded. Missing data on the date of admission was assigned a normal value and missing data after the day one was substituted by carrying forward the previous day's value. Demographic and microbiologic data were recorded. When data collection for each subject was complete, all subject identifiers were removed from all records and the subject file was assigned a unique random number that was cross referenced with the to blood samples. The completed raw data file was converted to calculated descriptive and severity of illness scores using standard definitions (i.e. APACHE II and Days alive and free of organ dysfunction calculated using the Brussels criteria).

Baseline characteristics (age, gender, admitting APACHE II score, and medical versus surgical admitting diagnosis) were recorded and compared across F3 SNPs and genotype groups using a chi-squared or Kruskal-Wallis test were conducted where appropriate. We then carried out Cox proportional hazards (CPH) regression using the survival and event history analysis packages in R (R Core Development Group, 2005) to assess whether the C4524T, C599T, A1089G, A1826G, and T13925C polymorphisms (chosen using the cladistic approach described above) were significantly associated with clinical outcomes among SIRS, sepsis, and septic shock subjects. Univariate models were constructed using either allele or genotype (additive, recessive, and dominant models) information. Multivariate models included tissue factors C4524T, C599T, A1089G, A1826G and T13925C and baseline characteristic variables as covariates.

We used a cohort study design. Rates of dichotomous outcomes (28-day mortality, sepsis and shock at onset of SIRS) were compared between haplotype clades using a chi-squared test, assuming a dominant model of inheritance. Differences in continuous outcome variables between haplotype clades were tested using ANOVA. 28-day mortality was further compared between haplotype clades while adjusting for other confounders (age, sex, and medical vs. surgical diagnosis) using a Cox regression model, together with Kaplan-Meier analysis. Haplotype clade relative risk was calculated. This analysis was performed in the entire cohort, and subsequently in sub-groups of subjects who had sepsis at onset of SIRS, and subjects who had septic shock at onset of SIRS. Genotype distributions were tested for Hardy-Weinberg equilibrium (GUO S W. and THOMPSON EA. (1992) 48:361-72). We report the mean and 95% confidence intervals. Statistical significance was set at p<0.05. The data was analyzed using SPSS 11.5 for Windows™ and SigmaStat 3.0 software (SPSS Inc, Chicago, Ill., 2003) and using statistical packages available in R (R Core Development Group, 2005—R Development Core Team (www.R-proiect.org). R: A language and environment for statistical computing. Vienna, Austria. 2005).

Hypertension

Tissue factor may be a key mediator of hypertension in diabetes, dyslipidemia, acute coronary syndromes, coronary artery disease, atherosclerosis, and pulmonary hypertension.

Interestingly, treatment with antihypertensive agents blocking the actions of angiotensin II receptor (i.e., ATGR1) decreases endothelial cell expression of F3 (MULLER D N et al., Am J Pathol (2000) 157:111-22). Similarly, treatment of hypercholesterolemic individuals with statins reduces hypertension and tissue factor levels (TSIARA S et al. Curr Med Res Opin (2003) 19(6):540-56).

### 3. Examples

### 3.1 Association of Factor III Haplotypes with Prediction of Subject Outcome

A cohort of 234 Caucasian subjects having systematic inflammatory response syndrome (SIRS) and acute lung injury and who were admitted to the Intensive Care Unit (ICU) of St. Paul's Hospital in Vancouver, BC. Canada were prospectively studied. Similarly, a cohort of 130 Asian subjects having SIRS and who were admitted to the Intensive Care Unit (ICU) of St. Paul's Hospital were prospectively studied.

Two types of analyses are shown in the following examples. The allele analyses are generated using alleles as the independent (predictive variables) in each analysis. These are obtained by splitting genotypes into alleles and “stacking” the data so that each person has two observations per locus. Accordingly, the allele sample sizes are double those of their genotype counterparts.

A recessive analysis is generated where the major homozygote and heterozygote are grouped together and compared to the minor homozygote. This analysis was termed “recessive”, because if the proper ordinal scores were assigned to each genotype group, it to would correspond to the recessive model under the alternative hypothesis that the rare allele was the causative variant.

In the Results below the abbreviations set our in the below Legend TABLES (A and B) are used.

### Legend A

Baseline Key

| | |
|---|---|
| AGE | Given In Years |
| GENDER | Percentage of Male Subjects |
| APACHE II | APACHE II score |
| % SURGICAL | The % of subjects who had a % |
| | SURGICAL ICU admitting diagnosis |
| SURVIVAL | 28-day survival |
| SEP.ADMIT | Sepsis upon admission |
| SEP.ANY | Sepsis anytime during admission |
| SS.ADMIT | Septic shock upon admission |
| SS.ANY | Septic shock anytime during admission |

Note.
X/X/X = 25%-ile/median/75%-ile

### Legend B

Days alive and free (DAF) of organ dysfunction Key

| | |
|---|---|
| SURVIVAL | 28 Day Survival |
| MSIRS4.DAF | Days Alive and Free of 4/4 SIRS Criteria |
| ALI.DAF | Days alive and free of Acute Lung Injury |
| PRESS.DAF | Days alive and free of Any vasopressors |
| PRESS2.DAF | Days alive and free of More than 2 ug/min |
| | of vasopressors |
| PRESS5.DAF | Days alive and free of More than 5 ug/min |
| | of vasopressors |
| PRESS15.DAF | Days alive and free of More than 15 ug/min |
| | of vasopressors |
| INO.DAF | Days alive and free of Inotropes |
| SIRS2.DAF | Days alive and free of 2 of 4 SIRS criteria |
| SIRS3.DAF | Days alive and free of 3 of 4 SIRS criteria |
| SIRS4.DAF | Days alive and free of 4 of 4 SIRS criteria |
| STER.DAF | Days alive and free of steroids |
| CVS.DAF | Days alive and free of Cardiovascular dysfunction |
| RESP.DAF | Days alive and free of Respiratory dysfunction |
| PF300.DAF | Days alive and free of PaO2/FiO2 less than 300 |
| VENT.DAF | Days alive and free of Mechanical Ventilators |
| CNS.DAF | Days alive and free of Neurological Dysfunction |
| COAG.DAF | Days alive and free of Coagulation Dysfunction |
| INR.DAF | Days alive and free of International normalized |
| | ratio >1.5 |
| ACRF.DAF | Days alive and free of Acute renal failure |
| ANYREN.DAF | Days alive and free of Any type of renal dysfunction |
| RENSUP.DAF | Days alive and free of Renal Support |
| ACHEP.DAF | Days alive and free of Acute hepatic dysfunction |
| ANYHEP.DAF | Days alive and free of Any type of hepatic dysfunction |
| AFFD.DAF | Days alive and free of Acute Failure free days |
| FFD.DAF | Days alive and free of Failure free days |
| | (Acute or Chronic) |

Note.
X/X/X = 25%-ile/median/75%-ile

3.1.1 Coagulation Factor III C4524T

i) Allele Analysis—Cohort of Caucasian Subjects Who Had SIRS and Acute Lung Injury

Of the Caucasians who had SIRS and acute lung injury, 234 were successfully genotyped for polymorphisms of coagulation factor III and were included in this analysis. The frequency of the genotypes is shown in TABLE 2. These alleles were in Hardy Weinberg equilibrium in our population (TABLE 2). There were no significant differences in baseline characteristics of subjects according to the coagulation factor III C4524T genotype (TABLE 2). Subjects had a similar distribution of age, gender, medical/surgical statues, APACHE II scores upon admission, sepsis upon admission, sepsis anytime, septic shock upon admission and septic shock anytime.

TABLE 2
Baseline characteristics of cohort of critically ill Caucasian subjects who had systematic
inflammatory response syndrome and Acute Lung Injury for tissue factor 4524 C/T
defined by allele.

| | C | T | Combined | Test |
|---|---|---|---|---|
| | (N = 255) | (N = 205) | (N = 460) | Statistic |
| AGE | 43/54/70 | 43/56/68 | 44/57/71 | F = 0.04, DF = 1.458, P = 0.839 |
| SEX | 61% (156) | 63% (130) | 62% (286) | X² = 0.24, DF = 1, P = 0.623 |
| APACHEII | 19.0/24.0/29.0 | 18.0/23.0/29.0 | 18.0/23.5/29.0 | F = 0.06, DF = 1.458, P = 0.805 |
| SURGICAL | 14% (35) | 15% (31) | 14% (66) | X² = 0.18, DF = 1, P = 0.671 |
| SEP.ADMIT | 88% (225) | 90% (185) | 89% (410) | X² = 0.47, DF = 1, P = 0.491 |
| SEP.ANY | 89% (228) | 92% (188) | 90% (416) | X² = 0.69, DF = 1, P = 0.405 |
| SS.ADMIT | 69% (175) | 63% (129) | 66% (304) | X² = 1.65, DF = 1, P = 0.199 |
| SS.ANY | 72% (183) | 70% (143) | 71% (326) | X² = 0.22, DF = 1, P = 0.637 |

Caucasian subjects who had SIRS and acute lung injury who carried the C allele of coagulation factor III C4524T had significantly more pulmonary dysfunction as reflected by the fewer days alive and free of PaO2/FiO2 less than 300 (p=0.00536) (TABLE 3). Caucasian subjects who had SIRS and acute lung injury who carried the C allele of coagulation factor III C4524T had significantly more need for renal support as reflected by fewer days alive and free of renal support (p=0.0349) (TABLE 3).

TABLE 3
Days alive and free (DAF) of organ dysfunction by 4524 C/T allele of tissue factor in
cohort of critically ill Caucasian subjects who had systematic inflammatory response
syndrome and Acute Lung Injury.

| | C | T | Combined | Test |
|---|---|---|---|---|
| | (N = 255) | (N = 205) | (N = 460) | Statistic |
| PF300.DAF | 0.00/0.00/2.00 | 0.00/0.00/5.00 | 0.00/0.00/3.25 | F = 7.83, DF = 1.458, P = 0.00536 |
| RENSUP.DAF | 4/25/2028 | 7/28/2028 | 4/28/2028 | F = 4.48, DF = 1.458, P = 0.0349 |

ii). Allele Analysis—Cohort of Caucasian Subjects Who Had Sepsis and Acute Lung Injury

Of the Caucasian subjects who had sepsis and acute lung injury, 205 were successfully genotyped for polymorphisms of coagulation factor III C4524T and were included in this analysis. The frequency of the genotypes is shown in TABLE 4. These alleles were in Hardy Weinberg equilibrium in our population (TABLE 4). There were no significant differences in baseline characteristics of subjects who had sepsis according to the coagulation factor III C4524T genotype (TABLE 4). Subjects had a similar distribution of age, gender, medical/surgical statues, and APACHE II scores upon admission.

TABLE 4
Baseline characteristics of cohort of critically ill Caucasian subjects who had sepsis and
Acute Lung Injury for tissue factor 4524 C/T defined by allele.

| | C | T | Combined | Test |
|---|---|---|---|---|
| | (N = 225) | (N = 185) | (N = 410) | Statistic |
| AGE | 42.0/54.0/69.0 | 42.0/53.0/67.0 | 43.0/56.0/69.3 | F = 0.06, DF = 1.408, P = 0.808 |
| SEX | 62% (139) | 64% (119) | 63% (258) | X² = 0.28, DF = 1, P = 0.595 |
| APACHEII | 19/24/29 | 18/24/29 | 18/24/29 | F = 0.01, DF = 1.408, P = 0.91 |
| SURGICAL | 12% (28) | 15% (28) | 14% (56) | X² = 0.62, DF = 1, P = 0.43 |
| SS.ADMIT | 78% (175) | 70% (129) | 74% (304) | X² = 3.43, DF = 1, P = 0.064 |
| SS.ANY | 81% (182) | 76% (140) | 79% (322) | X² = 1.64, DF = 1, P = 0.201 |

Caucasian subjects who had sepsis and acute lung injury who carried the C allele of coagulation factor III C4524T had significantly more acute lung injury as reflected by the fewer days alive and free of acute lung injury (p=0.0506), significantly more respiratory dysfunction as reflected by fewer days alive and free of respiratory dysfunction (p=0.0437), and significantly greater need for mechanical ventilation as reflected by fewer days alive and free of ventilation (p=0.0337) (TABLE 5). Caucasian subjects who had sepsis and acute lung injury who carried the C allele of coagulation factor III C4524T also had significantly more cardiovascular dysfunction as reflected by fewer days alive and free of vasopressors (p=0.0407), significantly more cardiovascular dysfunction as reflected by fewer days alive and free of cardiovascular dysfunction (p=0.0426) and had significantly more neurologic dysfunction as reflected by fewer days alive and free of neurologic dysfunction (p=0.0293) (Brussels criteria TABLE 1F) (TABLE 5). Caucasian subjects who had sepsis and acute lung injury who carried the C allele of coagulation factor III C4524T had significantly greater need for renal support as shown by fewer days alive and free of renal support (p=0.0308) (TABLE 5). Thus, Caucasian subjects who had sepsis and acute lung injury who carried the C allele of coagulation factor III C4524T had more acute lung injury, more respiratory dysfunction, more cardiovascular dysfunction, more neurological dysfunction and greater need for renal support.

TABLE 5
Days alive and free (DAF) of organ dysfunction by 4524 C/T allele of tissue factor in
cohort of critically ill Caucasian subjects who had sepsis and Acute Lung Injury.

| | C | T | Combined | Test |
|---|---|---|---|---|
| | (N = 225) | (N = 185) | (N = 410) | Statistic |
| ALI.DAF | 0/3/20 | 1/7/2021 | 0/5/20 | F = 3.84, DF = 1.408, P = 0.0506 |
| PRESS.DAF | 3.00/22.00/26.00 | 9.00/23.00/28.00 | 5.75/23.00/27.00 | F = 4.22, DF = 1.408, P = 0.0407 |
| CVS.DAF | 2/15/2025 | 5/19/2026 | 3/17/2025 | F = 4.14, DF = 1.408, P = 0.0426 |
| RESP.DAF | 0.0/6.0/22.0 | 0.0/14.0/24.0 | 0.0/8.5/22.3 | F = 4.09, DF = 1.408, P = 0.0437 |
| PF300.DAF | 0/0/2 | 0/1/7 | 0/0/4 | F = 7.84, DF = 1.408, P = 0.00536 |
| VENT.DAF | 0.0/5.0/22.0 | 0.0/14.0/23.0 | 0.0/7.5/22.0 | F = 4.54, DF = 1.408, P = 0.0337 |
| CNS.DAF | 6/24/2028 | 9/26/2028 | 7/25/2028 | F = 4.78, DF = 1.408, P = 0.0293 |

iii). Allele Analysis—Cohort of Caucasian Subjects Who Had Septic Shock and Acute Lung Injury

Of the Caucasian subjects who had septic shock, 152 were successfully genotyped for polymorphisms of coagulation factor III C4524T and were included in this analysis. The frequency of the genotypes is shown in TABLE 6. These alleles were in Hardy Weinberg equilibrium in our population (TABLE 6). There were no significant differences in baseline characteristics of subjects who had sepsis according to the coagulation factor III C4524T genotype (TABLE 6). Subjects were of similar age, similar gender distribution, and had similar admitting APACHE II scores.

TABLE 6
Baseline characteristics of cohort of critically ill Caucasian subjects who had septic shock
and Acute Lung Injury for tissue factor 4524 C/T defined by allele.

| | C | T | Combined | Test |
|---|---|---|---|---|
| | (N = 175) | (N = 129) | (N = 304) | Statistic |
| AGE | 43.5/54.0/71.0 | 45.0/57.0/69.0 | 44.0/57.0/71.0 | F = 0.08, DF = 1.302, P = 0.775 |
| SEX | 61%(106) | 64%(82) | 62%(188) | X² = 0.28, DF = 1, P = 0.595 |
| APACHEII | 21/26/31 | 20/26/32 | 21/26/32 | F = 0.0, DF = 1.302, P = 0.915 |
| SURGICAL | 13%(23) | 19%(25) | 16%(48) | X² = 2.17, DF = 1, P = 0.140 |

Caucasian subjects who had septic shock and acute lung injury who were CC homozygotes of coagulation factor III C4524T had significantly more acute lung injury as reflected by the fewer days alive and free of acute lung injury (p=0.019) and significantly more respiratory dysfunction as reflected by fewer days alive and free of respiratory dysfunction (p=0.0223) and had significantly greater need for mechanical ventilation as shown by fewer days alive and free of ventilation (p=0.0192) (Brussels criteria, TABLE 1) (TABLE 7). Thus, Caucasian subjects who had septic shock and acute lung injury who were CC homozygotes had more acute lung injury and greater need for ventilation (TABLE 7).

TABLE 7
Days alive and free (DAF) of organ dysfunction by 4524 C/T allele of tissue factor in
cohort of critically ill Caucasian subjects who had septic shock and Acute Lung Injury.

| | C | T | Combined | Test |
|---|---|---|---|---|
| | (N = 175) | (N = 129) | (N = 304) | Statistic |
| ALI.DAF | 0.0/2.0/17.5 | 1.0/6.0/19.0 | 0.0/4.0/18.0 | F = 2.91, DF = 1.302, P = 0.089 |
| RESP.DAF | 0/3/21 | 0/8/22 | 0/5/21 | F = 2.6, DF = 1.302, P = 0.108 |
| VENT.DAF | 0/1/20 | 0/7/22 | 0/3/21 | F = 3.08, DF = 1.302, P = 0.08 |
| CNS.DAF | 4/22/2027 | 6/25/2028 | 5/24/2027 | F = 3.03, DF = 1.302, P = 0.0827 |

iv). Recessive Analysis—Cohort of Caucasian Subjects Who Had SIRS and Acute Lung Injury

Of the Caucasian subjects who had SIRS and acute lung injury, 230 were successfully genotyped for polymorphisms of coagulation factor III C4524T and were included in this analysis. The frequency of the genotypes (CC vs TT/CT) is shown in TABLE 8. There were no significant differences in baseline characteristics of subjects who had SIRS according to the coagulation factor III 4524 CC genotype vs. the 4524 TT/CT genotypes (TABLE 8). Subjects had a similar distribution of age, gender, medical/surgical statues, APACHE II scores upon admission, sepsis upon admission, sepsis anytime, septic shock upon admission and septic shock anytime.

TABLE 8
Baseline characteristics of cohort of critically ill Caucasian subjects who had systematic
inflammatory response syndrome and Acute Lung Injury by genotype of tissue factor 4524
C/T (CC vs. TT/CT).

| | CC | TT/CT | Combined | Test |
|---|---|---|---|---|
| | (N = 78) | (N = 152) | (N = 230) | Statistic |
| AGE | 44.3/59.5/72.8 | 42.0/54.0/68.0 | 44.0/57.0/71.0 | F = 1.5, DF = 1.228, P = 0.221 |
| SEX | 59% (46) | 64% (97) | 62% (143) | X² = 0.51, DF = 1, P = 0.474 |
| APACHEII | 19.3/25.0/30.8 | 18.0/23.0/29.0 | 18.0/23.5/29.0 | F = 1.2, DF = 1.228, P = 0.275 |
| SURGICAL | 12% (9) | 16% (24) | 14% (33) | X² = 0.76, DF = 1, P = 0.384 |
| SEP.ADMIT | 87% (68) | 90% (137) | 89% (205) | X² = 0.46, DF = 1, P = 0.496 |
| SEP.ANY | 88% (69) | 91% (139) | 90% (208) | X² = 0.53, DF = 1, P = 0.466 |
| SS.ADMIT | 73% (57) | 62% (95) | 66% (152) | X² = 2.57DF = 1, P = 0.109 |
| SS.ANY | 74% (58) | 69% (105) | 71% (163) | X² = 0.7, DF = 1, P = 0.404 |

Caucasian subjects who had SIRS and acute lung injury who were homozygous CC for the coagulation factor III C4524T had significantly more respiratory dysfunction as reflected by significantly fewer days alive and free of respiratory dysfunction (p=0.0115) than subjects who were coagulation factor III 4524 TT/CT. (TABLE 9).

TABLE 9
Days alive and free (DAF) of organ dysfunction by 4524 C/T
genotype (CC vs. TT/CT) of tissue factor in cohort of critically
ill Caucasian subjects who had systematic inflammatory
response syndrome and Acute Lung Injury.

| | CC | TT/CT | Combined | Test |
|---|---|---|---|---|
| | (N = 78) | (N = 152) | (N = 230) | Statistic |
| RESP.DAF | 0.0/5.5/22.8 | 0.0/13.5/24.0 | 0.0/9.0/23.0 | F = 1.97, |
| | | | | DF = 1.228, |
| | | | | P = 0.162 |

v). Recessive Analysis—Cohort of Caucasian Subjects Who Had Sepsis and Acute Lung Injury

Of the Caucasian subjects who had sepsis, 205 were successfully genotyped for polymorphisms of coagulation factor III C4524T and were included in this analysis. The frequency of the genotypes (CC vs TT/CT) is shown in TABLE 10. There were no significant differences in baseline characteristics of subjects who had Sepsis according to the coagulation factor III C4524T CC genotype vs. the TT/CT genotypes (TABLE 10). Subjects had a similar distribution of age, gender, medical/surgical statues, APACHE II scores upon admission, septic shock upon admission and septic shock anytime.

TABLE 10
Baseline characteristics of cohort of critically ill Caucasian subjects who had sepsis and
Acute Lung Injury by genotype of tissue factor 4524 C/T (CC vs. TT/CT).

| | CC | TT/CT | Combined | Test |
|---|---|---|---|---|
| | (N = 68) | (N = 137) | (N = 205) | Statistic |
| AGE | 44.0/56.0/71.3 | 41.0/53.0/68.0 | 43.0/56.0/69.3 | F = 1.14, DF = 1.203, P = 0.286 |
| SEX | 59% (40) | 65% (89) | 63% (129) | X² = 0.73, DF = 1, P = 0.391 |
| APACHEII | 20.8/25.0/30.3 | 18.0/24.0/29.0 | 18.0/24.0/29.0 | F = 0.96, DF = 1.203, P = 0.327 |
| SURGICAL | 10% (7) | 15% (21) | 14% (28) | X² = 0.98, DF = 1, P = 0.323 |
| SS.ADMIT | 84% (57) | 69% (95) | 74% (152) | X² = 4.97, DF = 1, P = 0.0258 |
| SS.ANY | 85% (58) | 75% (103) | 79% (161) | X² = 2.76, DF = 1, P = 0.0969 |

Caucasian subjects who had sepsis and acute lung injury who were homozygous for the coagulation factor III C4524T C allele (CC) had significantly more acute lung injury as reflected by the fewer days alive and free of acute lung injury (p=0.0202), significantly fewer days alive and free of respiratory dysfunction (p=0.0155) and significantly greater need for mechanical ventilation (p=0.0131) (TABLE 11) than subjects who were coagulation factor III C4524T TT/CT. Caucasian subjects who had sepsis and acute lung injury who were homozygous for the coagulation factor III C4524T C allele (CC) had significantly more need for vasopressors as reflected by the fewer days alive and free of vasopressors (p=0.0245) and significantly fewer days alive and free of cardiovascular dysfunction (p=0.0277) than subjects who were coagulation factor III C4524T TT/CT (TABLE 11). Caucasian subjects who had sepsis and acute lung injury who were homozygous for the coagulation factor III C4524T C allele (CC) had significantly more neurologic dysfunction as reflected by fewer days alive and free of neurologic dysfunction (p=0.0441) and significantly more need for renal support as reflected by the fewer days alive and free of renal support (p=0.0458) than subjects who were coagulation factor III C4524T TT/CT (TABLE 11). Thus Caucasian subjects who had sepsis and acute lung injury who were homozygous for the coagulation factor III C4524T C allele (CC) had significantly more acute lung injury, respiratory dysfunction, more need for ventilation, more cardiovascular dysfunction, greater need for vasopressors, more need for renal support, and more neurological dysfunction.

TABLE 11
Days alive and free (DAF) of organ dysfunction by 4524 C/T genotype (CC vs. TT/CT) of
tissue factor in cohort of critically ill Caucasian subjects who had sepsis and Acute Lung
Injury.

| | CC | TT/CT | Combined | Test |
|---|---|---|---|---|
| | (N = 68) | (N = 137) | (N = 205) | Statistic |
| ALI.DAF | 0.0/1.5/17.0 | 1.0/7.0/21.0 | 0.0/5.0/20.0 | F = 5.48, DF = 1.203, P = 0.0202 |
| PRESS.DAF | 2.00/19.50/25.00 | 9.00/24.00/28.00 | 5.75/23.00/27.00 | F = 5.13, DF = 1.203, P = 0.0245 |
| CVS.DAF | 1.0/12.5/24.0 | 5.0/19.0/26.0 | 3.0/17.0/25.0 | F = 4.92, DF = 1.203, P = 0.0277 |
| RESP.DAF | 0.0/2.0/21.0 | 0.0/14.0/24.0 | 0.0/8.5/22.3 | F = 5.96, DF = 1.203, P = 0.0155 |
| VENT.DAF | 0.0/0.5/20.3 | 0.0/14.0/23.0 | 0.0/7.5/22.0 | F = 6.27, DF = 1.203, P = 0.0131 |
| CNS.DAF | 4/23/2028 | 9/26/2028 | 7/25/2028 | F = 4.1, DF = 1.203, P = 0.0441 |
| RENSUP.DAF | 4.0/22.5/28.0 | 9.0/28.0/28.0 | 5.0/28.0/28.0 | F = 4.04, DF = 1.203, P = 0.0458 |

vi.) Recessive Analysis—Cohort of Caucasian Subjects Who Had Septic Shock and Acute Lung Injury

Of the Caucasian subjects who had septic shock and acute lung injury, 152 were successfully genotyped for polymorphisms of coagulation factor III C4524T and were included in this analysis. The frequency of the genotypes is shown in TABLE 12. There were no significant differences in baseline characteristics of subjects who had septic shock according to the coagulation factor III C4524T CC genotype vs. the TT/CT genotype (TABLE 12). Subjects were of similar age, similar gender distribution, and had similar admitting APACHE II scores.

TABLE 12
Baseline characteristics of cohort of critically ill Caucasian subjects who had septic shock
and Acute Lung Injury by genotype of tissue factor 4524 C/T (CC vs. TT/CT).

| | CC | TT/CT | Combined | Test |
|---|---|---|---|---|
| | (N = 57) | (N = 95) | (N = 152) | Statistic |
| AGE | 44.0/54.0/71.0 | 43.0/56.0/69.5 | 44.0/57.0/71.0 | F = 0.03, DF = 1.150, P = 0.86 |
| SEX | 56% (32) | 65% (62) | 62% (94) | X² = 1.26, DF = 1, P = 0.262 |
| APACHEII | 21.0/26.0/32.0 | 20.5/26.0/30.0 | 21.0/26.0/32.0 | F = 0.67, DF = 1.150, P = 0.414 |
| SURGICAL | 11% (6) | 19% (18) | 16% (24) | X² = 1.9, DF = 1, P = 0.168 |

Caucasian subjects who had septic shock and acute lung injury who were homozygous for the coagulation factor III C4524T C allele (CC) had significantly more acute lung injury as reflected by the fewer days alive and free of acute lung injury (p=0.019), significantly fewer days alive and free of respiratory dysfunction (p=0.0223) and significantly greater need for mechanical ventilation (p=0.0192) (TABLE 13) than subjects who were coagulation factor III C4524T TT/CT. Caucasian subjects who had septic shock and acute lung injury who were homozygous for the coagulation factor III C4524T C allele (CC) had strong trend to more need for vasopressors as reflected by the fewer days alive and free of vasopressors (p=0.0926) than subjects who were coagulation factor III C4524T TT/CT (TABLE 13). Thus Caucasian subjects who had septic shock and acute lung injury who were homozygous for the coagulation factor III C4524T C allele (CC) had significantly more acute lung injury, respiratory dysfunction, more need for ventilation, and greater need for vasopressors.

TABLE 13
Days alive and free (DAF) of organ dysfunction by 4524 C/T
genotype (CC vs. TT/CT) of tissue factor in cohort of critically ill
Caucasian subjects who had septic shock and Acute
Lung Injury.

| | CC | TT/CT | Combined | Test |
|---|---|---|---|---|
| | (N = 57) | (N = 95) | (N = 152) | Statistic |
| ALI.DAF | 0/1/10 | 1/6/2021 | 0/4/18 | F = 5.63, |
| | | | | DF = 1.150, |
| | | | | P = 0.0190 |
| PRESS2.DAF | 1/18/2024 | 6/22/2026 | 2/21/2026 | F = 2.86, |
| | | | | DF = 1.150, |
| | | | | P = 0.0926 |
| RESP.DAF | 0/1/17 | 0/8/22 | 0/5/21 | F = 5.33, |
| | | | | DF = 1.150, |
| | | | | P = 0.0223 |
| VENT.DAF | 0/0/17 | 0/7/22 | 0/3/21 | F = 5.6, |
| | | | | DF = 1.150, |
| | | | | P = 0.0192 |

3.1.2 Coagulation Factor III C599T

i). Allele Analysis—Cohort of Asian Subjects Who Had SIRS

Of the Asian who had SIRS, 246 were successfully genotyped for polymorphisms of coagulation factor III 599 C/T and were included in this analysis. The frequency of the genotypes is shown in TABLE 14. These alleles were in Hardy Weinberg equilibrium in our population (TABLE 14). There were no significant differences in baseline characteristics of subjects according to the coagulation factor III 599 C/T genotype (TABLE 14). Subjects had a similar distribution of age, gender, medical/surgical statues, APACHE II scores upon admission, sepsis upon admission, sepsis anytime, septic shock upon admission and septic shock anytime.

TABLE 14
Baseline characteristics of cohort of critically ill Asian subjects who had systematic
inflammatory response syndrome for tissue factor 599 C/T defined by allele.

| | T | C | Combined | Test |
|---|---|---|---|---|
| | (N = 54) | (N = 192) | (N = 246) | Statistic |
| AGE | 58.0/69.0/76.0 | 49.8/67.0/75.3 | 53.8/68.0/76.0 | F = 1.59, DF = 1.244, P = 0.209 |
| SEX | 74% (40) | 58% (112) | 62% (152) | X² = 4.42, DF = 1, P = 0.0355 |
| APACHEII | 17/23/29 | 18/23/30 | 17/23/31 | F = 0.39, DF = 1.244, P = 0.531 |
| SURGICAL | 24% (13) | 22% (43) | 23% (56) | X² = 0.07, DF = 1, P = 0.795 |
| SEP.ADMIT | 78% (42) | 79% (152) | 79% (194) | X² = 0.05, DF = 1, P = 0.825 |
| SEP.ANY | 85% (46) | 80% (154) | 81% (200) | X² = 0.69, DF = 1, P = 0.407 |
| SS.ADMIT | 52% (28) | 59% (114) | 58% (142) | X² = 0.98, DF = 1, P = 0.323 |
| SS.ANY | 63% (34) | 68% (130) | 67% (164) | X² = 0.43, DF = 1, P = 0.513 |

Asian subjects who had SIRS and carried the coagulation factor III 599 C allele had lower survival than Asian subjects who had SIRS and carried the coagulation factor III 599 T allele (survival: C=52%, T=65%, P=0.084). Asian subjects who had SIRS and carried the coagulation factor III 599 C allele also had more acute lung injury as reflected by the fewer days alive and free of acute lung injury (p=0.0622), more respiratory dysfunction as reflected by fewer days alive and free of respiratory dysfunction (p=0.0729) and significantly greater need for mechanical ventilation as reflected by fewer days alive and free of ventilation (p=0.0471)(TABLE 15). Asian subjects who had SIRS who carried the coagulation factor III 599 C allele had significantly more cardiovascular dysfunction as reflected by fewer days alive and free of vasopressors (p=0.0526), fewer days alive and free of neurological dysfunction (p=0.092), and fewer days alive and free of cardiovascular dysfunction (0.0671) (TABLE 15). Asian subjects who had SIRS who carried the coagulation factor III 599 C allele had more coagulopathy as shown by fewer days alive and free of coagulation dysfunction (p=0.0954) (TABLE 15). Asian subjects who had SIRS who carried the coagulation factor III 599 C allele had a strong trend to more acute renal dysfunction as reflected by fewer days alive and free of any renal dysfunction (p=0.0744) (TABLE 15). Asian subjects who had SIRS who carried the coagulation factor III 599 C allele had significantly more severe systemic inflammatory response as reflected by fewer days alive and free of 4 of 4 SIRS criteria (p=0.0467) (TABLE 15). Thus Asian subjects who had SIRS who carried the coagulation factor III 599 C allele had more acute lung injury, more respiratory dysfunction, more need for ventilation, more cardiovascular dysfunction and need for cardiovascular support, more coagulation dysfunction, more renal dysfunction, and more severe systemic inflammatory response (TABLE 15).

TABLE 15
Days alive and free (DAF) of organ dysfunction by 599 C/T allele of tissue factor in
cohort of critically ill Asian subjects who had systematic inflammatory response
syndrome.

| | T | C | Combined | Test |
|---|---|---|---|---|
| | (N = 54) | (N = 192) | (N = 246) | Statistic |
| SURV | 65% (35) | 52% (99) | 54% (134) | X² = 2.98, DF = 1, P = 0.084 |
| ALI.DAF | 6.0/25.5/28.0 | 2.0/12.5/28.0 | 2.0/13.0/28.0 | F = 3.51, DF = 1.244, P = 0.0622 |
| PRESS.DAF | 7.25/22.00/28.00 | 2.00/17.00/28.00 | 2.00/17.50/28.00 | F = 3.31, DF = 1.244, P = 0.07 |
| MSIRS4.DAF | 9.25/27.00/28.00 | 3.00/22.00/28.00 | 4.00/22.50/28.00 | F = 3.99, DF = 1.244, P = 0.0467 |
| CVS.DAF | 5.5/14.5/26.8 | 1.0/9.0/26.0 | 1.0/9.0/26.0 | F = 3.38, DF = 1.244, P = 0.0671 |
| RESP.DAF | 3.5/20.5/27.0 | 0.0/6.5/26.3 | 0.0/8.5/26.0 | F = 3.24, DF = 1.244, P = 0.0729 |
| VENT.DAF | 0.0/16.5/27.0 | 0.0/3.0/26.0 | 0.0/5.0/26.0 | F = 3.98, DF = 1.244, P = 0.0471 |
| CNS.DAF | 9.00/27.00/28.00 | 3.75/19.50/28.00 | 3.00/22.00/28.00 | F = 2.86, DF = 1.244, P = 0.092 |
| COAG.DAF | 9.25/24.50/28.00 | 3.00/18.00/28.00 | 3.00/20.00/28.00 | F = 2.8, DF = 1.244, P = 0.0954 |
| ANYREN.DAF | 0.25/11.50/28.00 | 0.00/3.00/25.50 | 0.00/4.50/28.00 | F = 3.21, DF = 1.244, P = 0.0744 |

ii) Allele Analysis—Cohort of Asian Subjects Who Had Sepsis

Of the Asian who had sepsis, 194 were successfully genotyped for polymorphisms of coagulation factor III 599 C/T and were included in this analysis. The frequency of the genotypes is shown in TABLE 16. These alleles were in Hardy Weinberg equilibrium in our population (TABLE 16). There were no significant differences in baseline characteristics of subjects according to the coagulation factor III 599 C/T genotype (TABLE 16). Subjects had a similar distribution of age, gender, medical/surgical statues, APACHE II scores upon admission, septic shock upon admission and septic shock anytime.

TABLE 16
Baseline characteristics of cohort of critically ill Asian subjects who had sepsis for tissue
factor 599 C/T defined by allele.

| | T | C | Combined | Test |
|---|---|---|---|---|
| | (N = 42) | (N = 152) | (N = 194) | Statistic |
| AGE | 65.3/73.0/76.0 | 54.0/68.0/76.0 | 56.8/68.0/76.0 | F = 2.82, DF = 1.192, P = 0.0947 |
| SEX | 81% (34) | 58% (88) | 63% (122) | X² = 7.5, DF = 1, P = 0.00618 |
| APACHEII | 17.3/24.0/29.8 | 18.0/23.0/32.0 | 18.0/23.0/32.0 | F = 0.12, DF = 1.192, P = 0.731 |
| SURGICAL | 24% (10) | 25% (38) | 25% (48) | X² = 0.03, DF = 1, P = 0.874 |
| SS.ADMIT | 67% (28) | 75% (114) | 73% (142) | X² = 1.16, DF = 1, P = 0.280 |
| SS.ANY | 76% (32) | 84% (128) | 82% (160) | X² = 1.46, DF = 1, P = 0.226 |

Asian subjects who had sepsis who carried the coagulation factor III 599 C allele had greater need for mechanical ventilation as reflected by fewer days alive and free of ventilation (p=0.0809) (TABLE 17). Asian subjects who had sepsis who carried the coagulation factor III 599 C allele had significantly more cardiovascular dysfunction as reflected by fewer days alive and free of cardiovascular dysfunction (0.0722) (TABLE 17). Asian subjects who had sepsis who carried the coagulation factor III 599 C allele had significantly greater need for steroids as shown by fewer days alive and free of steroids (p=0.0083) (TABLE 17). Thus Asian subjects who had sepsis who carried the coagulation factor III 599 C allele had more need for ventilation, more cardiovascular dysfunction and a significantly greater need for steroids (TABLE 17).

TABLE 17
Days alive and free (DAF) of organ dysfunction by 599 C/T allele of tissue factor in
cohort of critically ill Asian subjects who had sepsis.

| | T | C | Combined | Test |
|---|---|---|---|---|
| | (N = 42) | (N = 152) | (N = 194) | Statistic |
| STER.DAF | 3.75/12.00/28.00 | 0.00/5.00/28.00 | 1.00/6.00/28.00 | F = 7.13, DF = 1.192, P = 0.00825 |
| CVS.DAF | 4.0/10.5/26.0 | 1.0/8.0/23.3 | 1.0/8.0/23.8 | F = 3.27, DF = 1.192, P = 0.0722 |
| VENT.DAF | 0.0/9.5/26.0 | 0.0/1.0/23.3 | 0.0/3.0/23.8 | F = 3.08, DF = 1.192, P = 0.0809 |

iii) Recessive Analysis—Cohort of Asian Subjects Who Had SIRS

Of the Asian subjects who had SIRS, 123 were successfully genotyped for polymorphisms of coagulation factor III 599 C/T and were included in this analysis. The frequency of the genotypes (CC/TC vs TT) is shown in TABLE 18. There were no significant differences in baseline characteristics of subjects who had SIRS according to the coagulation factor III 599 CC/TC genotypes vs. the TT genotype (TABLE 18). Subjects had a similar distribution of age, gender, medical/surgical statues, APACHE II scores upon admission, sepsis upon admission, sepsis anytime, septic shock upon admission and septic shock anytime.

TABLE 18
Baseline characteristics of cohort of critically ill Asian subjects who had systematic
inflammatory response syndrome by genotype of tissue factor 599 C/T (TT vs. CC/TC).

| | TT | CC/TC | Combined | Test |
|---|---|---|---|---|
| | (N = 7) | (N = 116) | (N = 123) | Statistic |
| AGE | 63.0/74.0/78.0 | 50.8/67.0/75.3 | 53.8/68.0/76.0 | F = 1.14, DF = 1.121 P = 0.289 |
| SEX | 71% (5) | 61% (71) | 62% (76) | X² = 0.29, DF = 1, P = 0.589 |
| APACHEII | 17.5/19.0/25.5 | 17.0/23.0/30.0 | 17.0/23.0/31.0 | F = 1.24, DF = 1.121, P = 0.267 |
| SURGICAL | 43% (3) | 22% (25) | 23% (28) | X² = 1.7, DF = 1, P = 0.192 |
| SEP.ADMIT | 86% (6) | 78% (91) | 79% (97) | X² = 0.21, DF = 1, P = 0.647 |
| SEP.ANY | 100% (7) | 80% (93) | 81% (100) | X² = 1.71, DF = 1, P = 0.191 |
| SS.ADMIT | 43% (3) | 59% (68) | 58% (71) | X² = 0.67, DF = 1, P = 0.412 |
| SS.ANY | 57% (4) | 67% (78) | 67% (82) | X² = 0.3, DF = 1, P = 0.582 |

Asian subjects who had SIRS who were either CC or TC for the coagulation factor III 599 SNP had lower survival (p=0.0878). Asian subjects were either CC or TC for the coagulation factor III 599 SNP had significantly more acute lung injury as reflected by the fewer days alive and free of acute lung injury (p=0.0194), fewer days alive and free of respiratory dysfunction (p=0.0644) and fewer days alive and free of mechanical ventilation (p=0.0899) (TABLE 19) than subjects who were coagulation factor III 599 TT. Asian subjects who had SIRS who were either CC or TC for the coagulation factor III 599 SNP had significantly fewer days alive and free of vasopressors as reflected by the fewer days alive and free of vasopressors (p=0.0136) and had significantly fewer days alive and free of cardiovascular dysfunction (p=0.0215) than subjects who were coagulation factor III 599 TT (TABLE 19). Asian subjects who had SIRS who were either CC or TC for the coagulation factor III 599 SNP had a significantly more coagulation dysfunction as reflected by fewer days alive and free of coagulation dysfunction (p=0.0117), and significantly fewer days alive and free of acute hepatic (p=0.0317) and of any hepatic dysfunction (p=0.0307) than subjects who were coagulation factor III 599 TT (TABLE 19). Asian subjects who had SIRS who were either CC or TC for the coagulation factor III 599 SNP had more neurological dysfunction as reflected by the fewer days alive and free of neurological dysfunction (p=0.0899) than subjects who were coagulation factor III 599 TT (TABLE 19). Asian subjects who had SIRS who were either CC or TC for the coagulation factor III 599 SNP had more severe SIRS as reflected by the fewer days alive and free of 4 of 4 SIRS criteria (p=0.0563) than subjects who were coagulation factor III 599 TT (TABLE 19). Asian subjects who had SIRS who were either CC or TC for the coagulation factor III 599 SNP had significantly greater need for steroids as shown by fewer days alive and free of steroids (p=0.0058) (TABLE 19). Thus subjects who were either CC or TC for the coagulation factor III 599 SNP and had SIRS had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 19
Days alive and free (DAF) of organ dysfunction by 599 C/T genotype (TT vs. CC/TC) of
tissue factor in cohort of critically ill Asian subjects who had systematic inflammatory
response syndrome.

| | TT | CC/TC | Combined | Test |
|---|---|---|---|---|
| | (N = 7) | (N = 116) | (N = 123) | Statistic |
| SURV | 86% (6) | 53% (61) | 54% (67) | X² = 2.92, DF = 1, P = 0.0874 |
| ALI.DAF | 28.0/28.0/28.0 | 2.0/12.5/28.0 | 2.0/13.0/28.0 | F = 5.61, DF = 1.121, P = 0.0194 |
| PRESS.DAF | 27.0/28.0/28.0 | 2.0/17.0/28.0 | 2.0/17.5/28.0 | F = 6.27, DF = 1.121, P = 0.0136 |
| MSIRS4.DAF | 27.0/27.0/28.0 | 4.0/22.0/28.0 | 4.0/22.5/28.0 | F = 3.71, DF = 1.121, P = 0.0563 |
| STER.DAF | 28.0/28.0/28.0 | 1.0/6.5/28.0 | 1.0/7.0/28.0 | F = 7.88, DF = 1.121, P = 0.00582 |
| CVS.DAF | 22/26/27 | 1/9/2026 | 1/9/2026 | F = 5.43, DF = 1.121, P = 0.0215 |
| RESP.DAF | 24.0/27.0/27.0 | 0.0/7.5/26.3 | 0.0/8.5/26.0 | F = 3.48, DF = 1.121, P = 0.0644 |
| VENT.DAF | 22.5/26.0/27.0 | 0.0/4.0/26.0 | 0.0/5.0/26.0 | F = 4.94, DF = 1.121, P = 0.0281 |
| CNS.DAF | 25.5/28.0/28.0 | 4.0/19.5/28.0 | 3.0/22.0/28.0 | F = 2.92, DF = 1.121, P = 0.0899 |
| COAG.DAF | 28.0/28.0/28.0 | 3.0/18.5/28.0 | 3.0/20.0/28.0 | F = 6.55, DF = 1.121, P = 0.0117 |
| ACHEP.DAF | 28.00/28.00/28.00 | 2.75/16.00/28.00 | 2.00/15.50/28.00 | F = 4.73, DF = 1.121, P = 0.0317 |
| ANYHEP.DAF | 28.0/28.0/28.0 | 2.0/16.0/28.0 | 2.0/15.5/28.0 | F = 4.78, DF = 1.121, P = 0.0307 |

iv) Recessive Analysis—Cohort of Asian Subjects Who Had Sepsis

Of the Asian subjects who had sepsis, 97 were successfully genotyped for polymorphisms of coagulation factor III 599 C/T and were included in this analysis. The frequency of the genotypes (CC/TC vs TT) is shown in TABLE 20. There were no significant differences in baseline characteristics of subjects who had sepsis according to the coagulation factor III 599 CC/TC genotypes vs. the TT genotype (TABLE 20). Subjects had a similar distribution of age, gender, medical/surgical statues, APACHE II scores upon admission, septic shock upon admission and septic shock anytime.

TABLE 20
Baseline characteristics of cohort of critically ill Asian subjects who had sepsis by
genotype of tissue factor 599 C/T (TT vs. CC/TC).

| | TT | CC/TC | Combined | Test |
|---|---|---|---|---|
| | (N = 6) | (N = 91) | (N = 97) | Statistic |
| AGE | 73.3/75.0/79.0 | 55.5/68.0/76.0 | 57.5/68.0/76.0 | F = 1.83, DF = 1.95, P = 0.179 |
| SEX | 83% (5) | 62% (56) | 63% (61) | X²2 = 1.15, DF = 1, P = 0.284 |
| APACHEII | 18.3/21.0/26.8 | 18.0/24.0/32.0 | 18.0/23.0/32.0 | F = 0.5, DF = 1.95, P = 0.479 |
| SURGICAL | 50% (3) | 23% (21) | 25% (24) | X² = 2.19, DF = 1, P = 0.139 |
| SS.ADMIT | 50% (3) | 75% (68) | 73% (71) | X² = 1.75, DF = 1, P = 0.185 |
| SS.ANY | 67% (4) | 84% (76) | 82% (80) | X² = 1.11, DF = 1, P = 0.293 |

Asian subjects who had sepsis who were either CC or TC for the coagulation factor III 599 SNP had lower survival (p=0.0776). Asian subjects who had sepsis who were either CC or TC for the coagulation factor III 599 SNP had significantly more acute lung injury as reflected by the fewer days alive and free of acute lung injury (p=0.0222), significantly fewer days alive and free of respiratory dysfunction (p=0.0463) and significantly fewer days alive and free of mechanical ventilation (p=0.0214) (TABLE 21) than subjects who were coagulation factor III 599 TT. Asian subjects who had sepsis who were either CC or TC for the coagulation factor III 599 SNP had significantly fewer days alive and free of vasopressors as reflected by the fewer days alive and free of vasopressors (p=0.0128) and had significantly fewer days alive and free of cardiovascular dysfunction (p=0.0073) than subjects who were coagulation factor III 599 TT (TABLE 21). Asian subjects who had sepsis who were either CC or TC for the coagulation factor III 599 SNP had a significantly more coagulation dysfunction as reflected by fewer days alive and free of coagulation dysfunction (p=0.0124), and significantly fewer days alive and free of acute hepatic (p=0.0331) and of any hepatic dysfunction (p=0.0318) than subjects who were coagulation factor III 599 TT (TABLE 21). Asian subjects who had sepsis who were either CC or TC for the coagulation factor III 599 SNP had more neurological dysfunction as reflected by the fewer days alive and free of neurological dysfunction (p=0.0573) than subjects who were coagulation factor III 599 TT (TABLE 21). Asian subjects who had sepsis who were either CC or TC for the coagulation factor III 599 SNP had significantly more severe SIRS as reflected by the fewer days alive and free of 4 of 4 SIRS criteria (p=0.0492) than subjects who were coagulation factor III 599 TT (TABLE 21). Asian subjects who had sepsis who were either CC or TC for the coagulation factor III 599 SNP had significantly greater need for steroids as shown by fewer days alive and free of steroids (p=0.0084) (TABLE 21). Thus subjects who were either CC or TC for the coagulation factor III 599 SNP and had sepsis had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 21
Days alive and free (DAF) of organ dysfunction by 599 C/T genotype (TT vs. CC/TC) of
tissue factor in cohort of critically ill Asian subjects who had sepsis.

| | TT | CC/TC | Combined | Test |
|---|---|---|---|---|
| | (N = 6) | (N = 91) | (N = 97) | Statistic |
| SURV | 83% (5) | 46% (42) | 48% (47) | X² = 3.12, DF = 1, P = 0.0776 |
| ALI.DAF | 28/28/28 | 2/9/2028 | 2/9/2028 | F = 5.41, DF = 1.95, P = 0.0222 |
| PRESS.DAF | 26.5/28.0/28.0 | 2.0/12.0/26.0 | 2.0/12.0/27.0 | F = 6.44, DF = 1.95, P = 0.0128 |
| MSIRS4.DAF | 27.0/27.0/27.8 | 3.0/16.0/27.0 | 3.5/18.0/27.0 | F = 3.97, DF = 1.95, P = 0.0492 |
| STER.DAF | 28/28/28 | 1/6/2028 | 1/6/2028 | F = 7.33, DF = 1.95, P = 0.00804 |
| CVS.DAF | 26.0/26.5/27.0 | 1.0/8.0/23.0 | 1.0/8.0/23.5 | F = 7.52, DF = 1.95, P = 0.0073 |
| RESP.DAF | 23.0/26.5/27.0 | 0.0/5.0/24.0 | 0.0/6.0/24.5 | F = 4.08, DF = 1.95, P = 0.0463 |
| VENT.DAF | 20.8/26.0/26.8 | 0.0/1.0/23.0 | 0.0/3.0/23.5 | F = 5.47, DF = 1.95, P = 0.0214 |
| CNS.DAF | 24.8/27.5/28.0 | 3.0/16.0/28.0 | 3.0/18.0/28.0 | F = 3.7, DF = 1.95, P = 0.0573 |
| COAG.DAF | 28/28/28 | 3/14/2028 | 3/16/2028 | F = 6.5, DF = 1.95, P = 0.0124 |
| ACHEP.DAF | 28/28/28 | 2/12/2028 | 2/12/2028 | F = 4.67, DF = 1.95, P = 0.0332 |
| ANYHEP.DAF | 28/28/28 | 2/12/2028 | 2/12/2028 | F = 4.75, DF = 1.95, P = 0.0318 |

v) Recessive Analysis—Cohort of Asian Subjects Who Had Septic Shock

Of the Asian subjects who had septic shock, 71 were successfully genotyped for polymorphisms of coagulation factor III 599 C/T and were included in this analysis. The frequency of the genotypes (CC/TC vs TT) is shown in TABLE 22. There were no significant differences in baseline characteristics of subjects who had septic shock according to the coagulation factor III 599 CC/TC genotypes vs. the TT genotype (TABLE 22). Subjects had a similar distribution of age, gender, medical/surgical statues and APACHE II scores upon admission.

TABLE 22
Baseline characteristics of cohort of critically ill Asian subjects who had septic shock by
genotype of tissue factor 599 C/T (TT vs. CC/TC).

| | TT | CC/TC | Combined | Test |
|---|---|---|---|---|
| | (N = 3) | (N = 68) | (N = 71) | Statistic |
| AGE | 54.0/76.0/80.0 | 59.8/68.0/76.0 | 61.5/68.0/76.0 | F = 0.21, DF = 1.69, P = 0.65 |
| SEX | 67% (2) | 63% (43) | 63% (45) | X² = 0.01, DF = 1, P = 0.904 |
| APACHEII | 20.5/23.0/25.5 | 21.8/26.5/32.3 | 21.0/26.0/32.5 | F = 0.78 DF = 1.69, P = 0.379 |
| SURGICAL | 67% (2) | 21% (14) | 23% (16) | X² = 3.49, DF = 1, P = 0.0616 |

Asian subjects who had septic shock who were either CC or TC for the coagulation factor III 599 SNP had lower survival (p=0.0575). Asian subjects who had septic shock who were either CC or TC for the coagulation factor III 599 SNP had significantly more acute lung injury as reflected by the fewer days alive and free of acute lung injury (p=0.0253), significantly fewer days alive and free of respiratory dysfunction (p=0.0404) and significantly fewer days alive and free of mechanical ventilation (p=0.0218) (TABLE 23) than subjects who were coagulation factor III 599 TT. Asian subjects who had septic shock who were either CC or TC for the coagulation factor III 599 SNP had significantly fewer days alive and free of vasopressors as reflected by the fewer days alive and free of vasopressors (p=0.0226) and had significantly fewer days alive and free of cardiovascular dysfunction (p=0.0132) than subjects who were coagulation factor III 599 TT (TABLE 23). Asian subjects who had septic shock who were either CC or TC for the coagulation factor III 599 SNP had a significantly more coagulation dysfunction as reflected by fewer days alive and free of coagulation dysfunction (p=0.0231), and significantly fewer days alive and free of acute hepatic (p=0.0426) and of any hepatic dysfunction (p=0.0426) than subjects who were coagulation factor III 599 TT (TABLE 23). Asian subjects who had septic shock who were either CC or TC for the coagulation factor III 599 SNP had more neurological dysfunction as reflected by the fewer days alive and free of neurological dysfunction (p=0.0557) than subjects who were coagulation factor III 599 TT (TABLE 23). Asian subjects who had septic shock who were either CC or TC for the coagulation factor III 599 SNP had more severe SIRS as reflected by the fewer days alive and free of 4 of 4 SIRS criteria (p=0.0613) than subjects who were coagulation factor III 599 TT (TABLE 23). Asian subjects who had septic shock who were either CC or TC for the coagulation factor III 599 SNP had significantly greater need for steroids as shown by fewer days alive and free of steroids (p=0.0247) (TABLE 23). Thus subjects who were either CC or TC for the coagulation factor III 599 SNP and had septic shock had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 23
Days alive and free (DAF) of organ dysfunction by 599 C/T genotype (TT vs. CC/TC) of
tissue factor in cohort of critically ill Asian subjects who had septic shock.

| | TT | CC/TC | Combined | Test |
|---|---|---|---|---|
| | (N = 3) | (N = 68) | (N = 71) | Statistic |
| SURV | 100% (3) | 44% (30) | 46% (33) | X² = 3.61 DF = 1, P = 0.0575 |
| ALI.DAF | 28.0/28.0/28.0 | 1.0/5.5/26.5 | 1.0/6.0/28.0 | F = 5.23, DF = 1.69, P = 0.0253 |
| PRESS.DAF | 27/28/28 | 1/10/2026 | 1/9/2026 | F = 5.44, DF = 1.69, P = 0.0226 |
| MSIRS4.DAF | 27.00/27.00/27.50 | 2.75/12.50/27.00 | 2.50/15.00/27.00 | F = 3.62, DF = 1.69, P = 0.0613 |
| STER.DAF | 28.00/28.00/28.00 | 0.75/3.50/27.25 | 1.00/5.00/28.00 | F = 5.27, DF = 1.69, P = 0.0247 |
| CVS.DAF | 26.0/26.0/26.5 | 0.0/4.0/22.3 | 0.0/4.0/22.5 | F = 6.48, DF = 1.69, P = 0.0132 |
| RESP.DAF | 26.5/27.0/27.0 | 0.0/1.0/23.3 | 0.0/1.0/24.0 | F = 4.36, DF = 1.69, P = 0.0404 |
| VENT.DAF | 26.5/27.0/27.0 | 0.0/0.5/21.5 | 0.0/1.0/22.5 | F = 5.51, DF = 1.69, P = 0.0218 |
| CNS.DAF | 27.5/28.0/28.0 | 3.0/13.0/27.3 | 3.0/15.0/27.5 | F = 3.79, DF = 1.69, P = 0.0557 |
| COAC.DAF | 28.00/28.00/28.00 | 2.75/12.50/26.25 | 2.00/13.00/28.00 | F = 5.4, DF = 1.69, P = 0.0231 |
| ACHEP.DAF | 28.0/28.0/28.0 | 2.0/11.0/28.0 | 1.5/9.0/28.0 | F = 4.27, DF = 1.69, P = 0.0426 |
| ANYHEP.DAF | 28.0/28.0/28.0 | 2.0/11.0/28.0 | 1.5/9.0/28.0 | F = 4.27, DF = 1.69, P = 0.0426 |

3.1.3 Coagulation factor III A1089G

i) Allele Analysis—Cohort of Asian Subjects Who Had SIRS

Asian subjects who had SIRS, 240 were successfully genotyped for polymorphisms of coagulation factor III 1089 A/G and were included in this analysis. The frequency of the genotypes is shown in TABLE 24. These alleles were in Hardy Weinberg equilibrium in our population (TABLE 24). There were no significant differences in baseline characteristics of subjects according to the coagulation factor III 1089 A/G genotype (TABLE 24). Subjects had a similar distribution of age, gender, medical/surgical statues, APACHE II scores upon admission, sepsis upon admission, sepsis anytime, septic shock upon admission and septic shock anytime.

TABLE 24
Baseline characteristics of cohort of critically ill Asian subjects who had systematic
inflammatory response syndrome for tissue factor 1089 A/G defined by allele.

| | G | A | Combined | Test |
|---|---|---|---|---|
| | (N = 187) | (N = 53) | (N = 240) | Statistic |
| AGE | 51.0/67.0/76.0 | 57.0/69.0/76.0 | 53.8/68.0/76.0 | F = 0.68, DF = 1.238, P = 0.412 |
| SEX | 57% (107) | 74% (39) | 61% (146) | X² = 4.64, DF = 1, P = 0.0312 |
| APACHEII | 18/23/31 | 17/23/29 | 17/23/31 | F = 0.74, DF = 1.238, P = 0.389 |
| SURGICAL | 23% (43) | 25% (13) | 23% (56) | X² = 0.05, DF = 1, P = 0.816 |
| SEP.ADMIT | 80% (149) | 77% (41) | 79% (190) | X² = 0.13, DF = 1, P = 0.713 |
| SEP.ANY | 81% (151) | 85% (45) | 82% (196) | X² = 0.48, DF = 1, P = 0.49 |
| SS.ADMIT | 60% (113) | 51% (27) | 58% (140) | X² = 1.53, DF = 1, P = 0.216 |
| SS.ANY | 68% (127) | 62% (33) | 67% (160) | X² = 0.59, DF = 1, P = 0.441 |

Asian subjects who had SIRS who carried the coagulation factor III 1089 G allele had lower survival than Asian subjects who had SIRS who carried the coagulation factor III 1089 A allele (survival: G=52%, A=66%, p=0.0673). Asian subjects who had SIRS who carried the coagulation factor III 1089 G allele had significantly more acute lung injury as reflected by the fewer days alive and free of acute lung injury (p=0.0376), more respiratory dysfunction as reflected by fewer days alive and free of respiratory dysfunction (p=0.0615) and significantly greater need for mechanical ventilation as reflected by fewer days alive and free of ventilation (p=0.0389) (TABLE 25). Asian subjects who had SIRS who carried the coagulation factor III 1089 G allele had more cardiovascular dysfunction as reflected by fewer days alive and free of vasopressors (p=0.0597), and significantly fewer days alive and free of cardiovascular dysfunction (0.053) (TABLE 25). Asian subjects who had SIRS who carried the coagulation factor III 1089 G allele had more coagulopathy as shown by fewer days alive and free of coagulation dysfunction (p=0.0824) (TABLE 25). Asian subjects who had SIRS who carried the coagulation factor III 1089 G allele had a strong trend to more renal dysfunction as reflected by fewer days alive and free of any renal dysfunction (p=0.0934) and a strong trend to more acute hepatic dysfunction as reflected by fewer days alive and free of acute hepatic dysfunction (p=0.0952) (TABLE 25). Asian subjects who had SIRS who carried the coagulation factor III 1089 G allele had significantly more severe systemic inflammatory response as reflected by fewer days alive and free of 4 of 4 SIRS criteria (p=0.0399) (TABLE 25). Asian subjects who had SIRS who carried the coagulation factor III 1089 G allele had significantly more need for steroid treatment as reflected by fewer days alive and free of steroids (p=0.0064) and more neurological dysfunction as reflected by fewer day alive and free of neurological dysfunction (p=0.0784) (TABLE 25) Thus Asian subjects who had SIRS who carried the coagulation factor III 1089 G allele had more acute lung injury, more respiratory dysfunction, more need for ventilation, more cardiovascular dysfunction and need for cardiovascular support, more coagulation dysfunction, more renal dysfunction, more severe systemic inflammatory response, more need for steroids and more neurological dysfunction (TABLE 25).

TABLE 25
Days alive and free (DAF) of organ dysfunction by 1089 G/A allele of tissue factor in
cohort of critically ill Asian subjects who had systematic inflammatory response
syndrome.

| | G | A | Combined | Test |
|---|---|---|---|---|
| | (N = 187) | (N = 53) | (N = 240) | Statistic |
| SURV | 52% (97) | 66% (35) | 55% (132) | X² = 3.35, DF = 1, P = 0.0673 |
| ALI.DAF | 1.5/13.0/28.0 | 6.0/26.0/28.0 | 2.0/13.0/28.0 | F = 4.37, DF = 1.238, P = 0.0376 |
| PRESS.DAF | 2.0/17.0/27.5 | 7.0/23.0/28.0 | 2.0/17.5/28.0 | F = 3.58, DF = 1.238, P = 0.0597 |
| MSIRS4.DAF | 3.5/22.0/28.0 | 9.0/27.0/28.0 | 4.0/22.5/28.0 | F = 4.27, DF = 1.238, P = 0.0399 |
| STER.DAF | 1/6/2028 | 6/18/2028 | 1/7/2028 | F = 7.55, DF = 1.238, P = 0.00648 |
| CVS.DAF | 0/9/26 | 5/15/2027 | 1/9/2026 | F = 3.78, DF = 1.238, P = 0.053 |
| RESP.DAF | 0.0/7.0/27.0 | 5.0/22.0/27.0 | 0.0/8.5/26.0 | F = 3.53, DF = 1.238, P = 0.0615 |
| VENT.DAF | 0/3/26 | 0/17/27 | 0/5/26 | F = 4.31, DF = 1.238, P = 0.0389 |
| CNS.DAF | 4/20/2028 | 9/27/2028 | 3/22/2028 | F = 3.12, DF = 1.238, P = 0.0784 |
| COAG.DAF | 3/18/2028 | 9/25/2028 | 3/20/2028 | F = 3.04, DF = 1.238, P = 0.0824 |
| ANYREN.DAF | 0.0/3.0/27.0 | 0.0/11.0/28.0 | 0.0/4.5/28.0 | F = 2.84, DF = 1.238, P = 0.0934 |
| ACHEP.DAF | 2.0/16.0/28.0 | 7.0/28.0/28.0 | 2.0/15.5/28.0 | F = 2.81, DF = 1.238, P = 0.0952 |

ii). Allele Analysis—Cohort of Asian Subjects Who Had Sepsis

Of the Asian who had sepsis, 190 were successfully genotyped for polymorphisms of coagulation factor III 1089 G/A and were included in this analysis. The frequency of the genotypes is shown in TABLE 26. These alleles were in Hardy Weinberg equilibrium in our population (TABLE 26). There were no significant differences in baseline characteristics of subjects according to the coagulation factor III 1089 G/A genotype (TABLE 26). Subjects had a similar distribution of age, gender, medical/surgical statues, APACHE II scores upon admission, septic shock upon admission and septic shock anytime.

TABLE 26
Baseline characteristics of cohort of critically ill Asian subjects who had
sepsis for tissue factor 1089 G/A defined by allele.

| | G | A | Combined | Test |
|---|---|---|---|---|
| | (N = 149) | (N = 41) | (N = 190) | Statistic |
| AGE | 55.0/68.0/76.0 | 65.0/73.0/76.0 | 56.8/68.0/76.0 | F = 1.68, DF = 1.188, P = 0.197 |
| SEX | 57% (85) | 80% (33) | 62% (118) | X² = 7.51, DF = 1, P = 0.00615 |
| APACHEII | 19/24/32 | 17/23/30 | 18/23/32 | F = 0.35, DF = 1.188, P = 0.556 |
| SURGICAL | 26% (38) | 24% (10) | 25% (48) | X² = 0.02, DF = 1, P = 0.885 |
| SS.ADMIT | 76% (113) | 66% (27) | 74% (140) | X² = 1.65, DF = 1, P = 0.199 |
| SS.ANY | 84% (125) | 76% (31) | 82% (156) | X² = 1.5, DF = 1, P = 0.220 |

Asian subjects who had sepsis who carried the coagulation factor III 1089 G allele had greater need for steroid treatment as reflected by fewer days alive and free of steroids (p=0.0086) (TABLE 27). Asian subjects who had sepsis who carried the coagulation factor III 1089 G allele had significantly more cardiovascular dysfunction as reflected by fewer days alive and free of cardiovascular dysfunction (0.0749) (TABLE 27). Asian subjects who had sepsis who carried the coagulation factor III 1089 G allele had greater need for ventilation as shown by fewer days alive and free of mechanical ventilation (p=0.0733) (TABLE 27). Thus Asian subjects who had sepsis who carried the coagulation factor III 1089 G allele had, more cardiovascular dysfunction, a significantly greater need for steroids and more need for mechanical ventilation (TABLE 27).

TABLE 27
Days alive and free (DAF) of organ dysfunction by 1089 G/A allele of
tissue factor in cohort of critically ill Asian subjects who had sepsis.

| | G | A | Combined | Test |
|---|---|---|---|---|
| | (N = 149) | (N = 41) | (N = 190) | Statistic |
| STER.DAF | 1/5/2028 | 6/12/2028 | 1/6/2028 | F = 7.06, DF = 1.188, P = 0.00858 |
| CVS.DAF | 1.0/8.0/24.0 | 4.0/11.0/26.0 | 1.0/8.0/23.8 | F = 3.21, DF = 1.188, P = 0.0749 |
| VENT.DAF | 0.0/1.0/24.0 | 0.0/10.0/26.0 | 0.0/3.0/23.8 | F = 3.24, DF = 1.188, P = 0.0733 |

iii). Recessive Analysis—Cohort of Asian Subjects Who Had SIRS

Of the Asian subjects who had SIRS, 120 were successfully genotyped for polymorphisms of coagulation factor III 1089 G/A and were included in this analysis. The frequency of the genotypes (GG/GA vs AA) is shown in TABLE 28. There were no significant differences in baseline characteristics of subjects who had SIRS according to the coagulation factor III 1089 GG/GA genotypes vs. the AA genotype (TABLE 28). Subjects had a similar distribution of age, gender, medical/surgical statues, APACHE II scores upon admission, sepsis upon admission, sepsis anytime, septic shock upon admission and septic shock anytime.

TABLE 28
Baseline characteristics of cohort of critically ill Asian subjects who had systematic
inflammatory response syndrome by genotype of tissue factor 1089 G/A
(GG/GA vs. AA).

| | GG/GA | AA | Combined | Test |
|---|---|---|---|---|
| | (N = 113) | (N = 7) | (N = 120) | Statistic |
| AGE | 51.0/67.0/76.0 | 63.0/74.0/78.0 | 53.8/68.0/76.0 | F = 1.01, DF = 1.118, P = 0.318 |
| SEX | 60% (68) | 71% (5) | 61% (73) | X² = 0.35, DF = 1, P = 0.554 |
| APACHEII | 17.0/23.0/31.0 | 17.5/19.0/25.5 | 17.0/23.0/31.0 | F = 1.39, DF = 1.118, P = 0.241 |
| SURGICAL | 22% (25) | 43% (3) | 23% (28) | X² = 1.58, DF = 1, P = 0.208 |
| SEP.ADMIT | 79% (89) | 86% (6) | 79% (95) | X² = 0.19, DF = 1, P = 0.66 |
| SEP.ANY | 81% (91) | 100% (7) | 82% (98) | X² = 1.67, DF = 1, P = 0.196 |
| SS.ADMIT | 59% (67) | 43% (3) | 58% (70) | X² = 0.73, DF = 1, P = 0.392 |
| SS.ANY | 67% (76) | 57% (4) | 67% (80) | X² = 0.3, DF = 1, P = 0.582 |

Asian subjects who had SIRS who were either GG or GA for the coagulation factor III 1089 SNP had lower survival (p=0.0923) than subjects who were coagulation factor III 1089 AA. Asian subjects were either GG or GA for the coagulation factor III 1089 SNP had significantly more acute lung injury as reflected by the fewer days alive and free of acute lung injury (p=0.0199), fewer days alive and free of respiratory dysfunction (p=0.0723) and significantly fewer days alive and free of mechanical ventilation (p=0.0316) (TABLE 29) than subjects who were coagulation factor III 1089 AA. Asian subjects who had SIRS who were either GG or GA for the coagulation factor III 1089 SNP had significantly fewer days alive and free of vasopressors as reflected by the fewer days alive and free of vasopressors (p=0.0138) and had significantly fewer days alive and free of cardiovascular dysfunction (p=0.0206) than subjects who were coagulation factor III 1089 AA (TABLE 29). Asian subjects who had SIRS who were either GG or GA for the coagulation factor III 1089 SNP had a significantly more coagulation dysfunction as reflected by fewer days alive and free of coagulation dysfunction (p=0.0116), and significantly fewer days alive and free of acute hepatic (p=0.0323) and of any hepatic dysfunction (p=0.0313) than subjects who were coagulation factor III 1089 AA (TABLE 29). Asian subjects who had SIRS who were either GG or GA for the coagulation factor III 1089 SNP had more neurological dysfunction as reflected by the fewer days alive and free of neurological dysfunction (p=0.0936) than subjects who were coagulation factor III 1089 AA (TABLE 29). Asian subjects who had SIRS were either GG or GA for the coagulation factor III 1089 SNP had more severe SIRS as reflected by the fewer days alive and free of 4 of 4 SIRS criteria (p=0.0566) than subjects who were coagulation factor III 1089 AA (TABLE 29). Asian subjects who had SIRS who were either GG or GA for the coagulation factor III 1089 SNP had significantly greater need for steroids as shown by fewer days alive and free of steroids (p=0.0058) (TABLE 29). Thus subjects who were either GG or GA for the coagulation factor III 1089 SNP and had SIRS had more acute lung injury, more respiratory dysfunction, more need for ventilation, more cardiovascular dysfunction, greater need for vasopressors, more coagulation dysfunction, more acute hepatic dysfunction, more neurological dysfunction, more severe SIRS and more need for steroids.

TABLE 29
Days alive and free (DAF) of organ dysfunction by 1089 G/A genotype (GG/GA vs. AA)
of tissue factor in cohort of critically ill Asian subjects who had systematic inflammatory
response syndrome.

| | GG/GA | AA | Combined | Test |
|---|---|---|---|---|
| | (N = 113) | (N = 7) | (N = 120) | Statistic |
| SURV | 53% (60) | 86% (6) | 55% (66) | X² = 2.83, DF = 1, P = 0.0923 |
| ALI.DAF | 2/13/2028 | 28/28/28 | 2/13/2028 | F = 5.57, DF = 1.118, P = 0.0199 |
| PRESS.DAF | 2.0/17.0/28.0 | 27.0/28.0/28.0 | 2.0/17.5/28.0 | F = 6.25, DF = 1.118, P = 0.0138 |
| MSIRS4.DAF | 4.0/22.0/28.0 | 27.0/27.0/28.0 | 4.0/22.5/28.0 | F = 3.71, DF = 1.118, P = 0.0566 |
| STER.DAF | 1/7/2028 | 28/28/28 | 1/7/2028 | F = 7.87, DF = 1.118, P = 0.00589 |
| CVS.DAF | 1/9/2026 | 22/26/27 | 1/9/2026 | F = 5.51, DF = 1.118, P = 0.0206 |
| RESP.DAF | 0.0/8.0/27.0 | 24.0/27.0/27.0 | 0.0/8.5/26.0 | F = 3.29, DF = 1.118, P = 0.0723 |
| VENT.DAF | 0.0/4.0/26.0 | 22.5/26.0/27.0 | 0.0/5.0/26.0 | F = 4.73, DF = 1.118, P = 0.0316 |
| CNS.DAF | 4.0/20.0/28.0 | 25.5/28.0/28.0 | 3.0/22.0/28.0 | F = 2.86, DF = 1.118, P = 0.0936 |
| COAG.DAF | 3/19/2028 | 28/28/28 | 3/20/2028 | F = 6.57, DF = 1.118, P = 0.0116 |
| ACHEP.DAF | 2.0/16.0/28.0 | 28.0/28.0/28.0 | 2.0/15.5/28.0 | F = 4.7, DF = 1.118, P = 0.0323 |
| ANYHEP.DAF | 2.0/16.0/28.0 | 28.0/28.0/28.0 | 2.0/15.5/28.0 | F = 4.75, DF = 1.118, P = 0.0313 |

iv). Recessive Analysis—Cohort of Asian Subjects Who Had Sepsis

Of the Asian subjects who had sepsis, 95 were successfully genotyped for polymorphisms of coagulation factor III 1089 G/A and were included in this analysis. The frequency of the genotypes (GG/GA vs. AA) is shown in TABLE 30. There were no significant differences in baseline characteristics of subjects who had sepsis according to the coagulation factor III 1089 GG/GA genotypes vs. the AA genotype (TABLE 30). Subjects had a similar distribution of age, gender, medical/surgical statues, APACHE II scores upon admission, septic shock upon admission and septic shock anytime.

TABLE 30
Baseline characteristics of cohort of critically ill Asian subjects who had sepsis by
genotype of tissue factor 1089 G/A (GG/GA vs. AA).

| | GG/GA | AA | Combined | Test |
|---|---|---|---|---|
| | (N = 89) | (N = 6) | (N = 95) | Statistic |
| AGE | 56.0/68.0/76.0 | 73.3/75.0/79.0 | 57.5/68.0/76.0 | F = 1.73, DF = 1.93, P = 0.192 |
| SEX | 61% (54) | 83% (5) | 62% (59) | X² = 1.23, DF = 1, P = 0.268 |
| APACHEII | 18.0/24.0/32.0 | 18.3/21.0/26.8 | 18.0/23.0/32.0 | F = 0.63, DF = 1.93, P = 0.429 |
| SURGICAL | 24% (21) | 50% (3) | 25% (24) | X² = 2.08, DF = 1, P = 0.150 |
| SS.ADMIT | 75% (67) | 50% (3) | 74% (70) | X² = 1.85, DF = 1, P = 0.173 |
| SS.ANY | 83% (74) | 67% (4) | 82% (78) | X² = 1.04, DF = 1, P = 0.308 |

Asian subjects who had sepsis who were either GG or GA for the coagulation factor III 1089 SNP had lower survival (p=0.0866) than subjects who were coagulation factor III 1089 AA. Asian subjects who had sepsis who were either GG or GA for the coagulation factor III 1089 SNP had significantly more acute lung injury as reflected by the fewer days alive and free of acute lung injury (p=0.0243), significantly fewer days alive and free of respiratory dysfunction (p=0.0528) and significantly fewer days alive and free of mechanical ventilation (p=0.0247) (TABLE 31) than subjects who were coagulation factor III 1089 AA. Asian subjects who had sepsis who were either GG or GA for the coagulation factor III 1089 SNP had significantly fewer days alive and free of vasopressors as reflected by the fewer days alive and free of vasopressors (p=0.0143) and had significantly fewer days alive and free of cardiovascular dysfunction (p=0.0081) than subjects who were coagulation factor III 1089 AA (TABLE 31). Asian subjects who had sepsis who were either GG or GA for the coagulation factor III 1089 SNP had a significantly more coagulation dysfunction as reflected by fewer days alive and free of coagulation dysfunction (p=0.0132), and significantly fewer days alive and free of acute hepatic (p=0.0357) and of any hepatic dysfunction (p=0.0342) than subjects who were coagulation factor III 1089 AA (TABLE 31). Asian subjects who had sepsis who were either GG or GA for the coagulation factor III 1089 SNP had more neurological dysfunction as reflected by the fewer days alive and free of neurological dysfunction (p=0.0643) than subjects who were coagulation factor III 1089 AA (TABLE 31). Asian subjects who had sepsis who were either GG or GA for the coagulation factor III 1089 SNP had significantly more severe SIRS as reflected by the fewer days alive and free of 4 of 4 SIRS criteria (p=0.0542) than subjects who were coagulation factor III 1089 AA (TABLE 31). Asian subjects who had sepsis who were either GG or GA for the coagulation factor III 1089 SNP had significantly greater need for steroids as shown by fewer days alive and free of steroids (p=0.0088) (TABLE 31). Thus subjects who were either GG or GA for the coagulation factor III 1089 SNP and had sepsis had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 31
Days alive and free (DAF) of organ dysfunction by 1089 G/A genotype (GG/GA vs. AA)
of tissue factor in cohort of critically ill Asian subjects who had sepsis.

| | GG/GA | AA | Combined | Test |
|---|---|---|---|---|
| | (N = 89) | (N = 6) | (N = 95) | Statistic |
| SURV | 47% (42) | 83% (5) | 49% (47) | X² = 2.94, DF = 1, P = 0.0866 |
| ALI.DAF | 2/9/2028 | 28/28/28 | 2/9/2028 | F = 5.24, DF = 1.93, P = 0.0243 |
| PRESS.DAF | 1.0/12.0/26.0 | 26.5/28.0/28.0 | 2.0/12.0/27.0 | F = 6.24, DF = 1.93, P = 0.0143 |
| MSIRS4.DAF | 3.0/16.0/27.0 | 27.0/27.0/27.8 | 3.5/18.0/27.0 | F = 3.8, DF = 1.93, P = 0.0542 |
| STER.DAF | 1/6/2028 | 28/28/28 | 1/6/2028 | F = 7.13, DF = 1.93, P = 0.00895 |
| CVS.DAF | 1.0/8.0/23.0 | 26.0/26.5/27.0 | 1.0/8.0/23.5 | F = 7.33, DF = 1.93, P = 0.00805 |
| RESP.DAF | 0.0/6.0/24.0 | 23.0/26.5/27.0 | 0.0/6.0/24.5 | F = 3.85, DF = 1.93, P = 0.0528 |
| VENT.DAF | 0.0/2.0/23.0 | 20.8/26.0/26.8 | 0.0/3.0/23.5 | F = 5.22, DF = 1.93, P = 0.0247 |
| CNS.DAF | 3.0/17.0/28.0 | 24.8/27.5/28.0 | 3.0/18.0/28.0 | F = 3.5, DF = 1.93, P = 0.0643 |
| COAG.DAF | 3/14/2028 | 28/28/28 | 3/16/2028 | F = 6.39, DF = 1.93, P = 0.0132 |
| ACHEP.DAF | 2/12/2028 | 28/28/28 | 2/12/2028 | F = 4.54, DF = 1.93, P = 0.0357 |
| ANYHEP.DAF | 2/12/2028 | 28/28/28 | 2/12/2028 | F = 4.62, DF = 1.93, P = 0.0342 |

v). Recessive Analysis—Cohort of Asian Subjects Who Had Septic Shock

Of the Asian subjects who had septic shock, 70 were successfully genotyped for polymorphisms of coagulation factor III 1089 G/A and were included in this analysis. The frequency of the genotypes (GG/GA vs. AA) is shown in TABLE 32. There were no significant differences in baseline characteristics of subjects who had septic shock according to the coagulation factor III 1089 GG/GA genotypes vs. the AA genotype (TABLE 32). Subjects had a similar distribution of age, gender, medical/surgical statues and APACHE II scores upon admission.

TABLE 32
Baseline characteristics of cohort of critically ill Asian subjects who had septic shock by
genotype of tissue factor 1089 G/A (GG/GA vs. AA).

| | GG/GA | AA | Combined | Test |
|---|---|---|---|---|
| | (N = 67) | (N = 3) | (N = 70) | Statistic |
| AGE | 58.5/68.0/76.0 | 54.0/76.0/80.0 | 61.5/68.0/76.0 | F = 0.19, DF = 1.68, P = 0.667 |
| SEX | 63% (42) | 67% (2) | 63% (44) | X² = 0.02, DF = 1 P = 0.889 |
| APACHEII | 21.5/27.0/32.5 | 20.5/23.0/25.5 | 21.0/26.0/32.5 | F = 0.86, DF = 1.68, P = 0.357 |
| SURGICAL | 21% (14) | 67% (2) | 23% (16) | X² = 3.41, DF = 1, P = 0.0647 |

Asian subjects who had septic shock who were either GG or GA for the coagulation factor III 1089 SNP had lower survival (p=0.0608) than subjects who were coagulation factor III 1089 AA. Asian subjects who had septic shock who were either GG or GA for the coagulation factor III 1089 SNP had significantly more acute lung injury as reflected by the fewer days alive and free of acute lung injury (p=0.026), significantly fewer days alive and free of respiratory dysfunction (p=0.0424) and significantly fewer days alive and free of mechanical ventilation (p=0.023) (TABLE 33) than subjects who were coagulation factor III 1089 AA. Asian subjects who had septic shock who were either GG or GA for the coagulation factor III 1089 SNP had significantly fewer days alive and free of vasopressors as reflected by the fewer days alive and free of vasopressors (p=0.0232) and had significantly fewer days alive and free of cardiovascular dysfunction (p=0.0132) than subjects who were coagulation factor III 1089 AA (TABLE 33). Asian subjects who had septic shock who were either GG or GA for the coagulation factor III 1089 SNP had a significantly more coagulation dysfunction as reflected by fewer days alive and free of coagulation dysfunction (p=0.0237), and significantly fewer days alive and free of acute hepatic (p=0.0439) and of any hepatic dysfunction (p=0.0439) than subjects who were coagulation factor III 1089 AA (TABLE 33). Asian subjects who had septic shock who were either GG or GA for the coagulation factor III 1089 SNP had more neurological dysfunction as reflected by the fewer days alive and free of neurological dysfunction (p=0.0578) than subjects who were coagulation factor III 1089 AA (TABLE 33). Asian subjects who had septic shock who were either GG or GA for the coagulation factor III 1089 SNP had more severe SIRS as reflected by the fewer days alive and free of 4 of 4 SIRS criteria (p=0.0635) than subjects who were coagulation factor III 1089 AA (TABLE 33). Asian subjects who had septic shock who were either GG or GA for the coagulation factor III 1089 SNP had significantly greater need for steroids as shown by fewer days alive and free of steroids (p=0.0256) (TABLE 33). Thus subjects who were either GG or GA for the coagulation factor III 1089 SNP and had septic shock had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 33
Days alive and free (DAF) of organ dysfunction by 1089 G/A genotype (GG/GA vs. AA)
of tissue factor in cohort of critically ill Asian subjects who had septic shock.

| | GG/GA | AA | Combined | Test |
|---|---|---|---|---|
| | (N = 67) | (N = 3) | (N = 70) | Statistic |
| SURV | 45% (30) | 100% (3) | 47% (33) | X² = 3.51, DF = 1, P = 0.0608 |
| ALI.DAF | 1/6/2027 | 28/28/28 | 1/6/2028 | F = 5.18, DF = 1.68, P = 0.026 |
| PRESS.DAF | 1/11/2026 | 27/28/28 | 1/9/2026 | F = 5.39, DF = 1.68, P = 0.0232 |
| MSIRS4.DAF | 2.0/13.0/27.0 | 27.0/27.0/27.5 | 2.5/15.0/27.0 | F = 3.56, DF = 1.68, P = 0.0635 |
| STER.DAF | 1.0/4.0/27.5 | 28.0/28.0/28.0 | 1.0/5.0/28.0 | F = 5.21, DF = 1.68, P = 0.0256 |
| CVS.DAF | 0.0/4.0/22.5 | 26.0/26.0/26.5 | 0.0/4.0/22.5 | F = 6.47, DF = 1.68, P = 0.0132 |
| RESP.DAF | 0.0/1.0/23.5 | 26.5/27.0/27.0 | 0.0/1.0/24.0 | F = 4.28, DF = 1.68, P = 0.0424 |
| VENT.DAF | 0.0/1.0/22.0 | 26.5/27.0/27.0 | 0.0/1.0/22.5 | F = 5.41, DF = 1.68, P = 0.023 |
| CNS.DAF | 2.5/13.0/27.5 | 27.5/28.0/28.0 | 3.0/15.0/27.5 | F = 3.73, DF = 1.68, P = 0.0578 |
| COAG.DAF | 2.0/12.0/26.5 | 28.0/28.0/28.0 | 2.0/13.0/28.0 | F = 5.35, DF = 1.68, P = 0.0237 |
| ACHEP.DAF | 2.0/11.0/28.0 | 28.0/28.0/28.0 | 1.5/9.0/28.0 | F = 4.22, DF = 1.68, P = 0.0439 |
| ANYHEP.DAF | 2.0/11.0/28.0 | 28.0/28.0/28.0 | 1.5/9.0/28.0 | F = 4.22, DF = 1.68, P = 0.0439 |

3.1.4 Coagulation factor III A1826G

i). Allele Analysis—Cohort of Asian Subjects Who Had SIRS

Of the Asian who had SIRS, 246 were successfully genotyped for polymorphisms of coagulation factor III 1826 A/G and were included in this analysis. The frequency of the genotypes is shown in TABLE 34. These alleles were in Hardy Weinberg equilibrium in our population (TABLE 34). There were no significant differences in baseline characteristics of subjects according to the coagulation factor III 1826 A/G genotype (TABLE 34). Subjects had a similar distribution of age, gender, medical/surgical statues, APACHE II scores upon admission, sepsis upon admission, sepsis anytime, septic shock upon admission and septic shock anytime.

TABLE 34
Baseline characteristics of cohort of critically ill Asian subjects who had systematic
inflammatory response syndrome for tissue factor 1826 A/G defined by allele.

| | G | A | Combined | Test |
|---|---|---|---|---|
| | (N = 54) | (N = 192) | (N = 246) | Statistic |
| AGE | 58.0/69.0/76.0 | 49.8/67.0/75.0 | 53.8/68.0/76.0 | F = 1.54, DF = 1.244, P = 0.216 |
| SEX | 74% (40) | 57% (110) | 61% (150) | X² = 4.99, DF = 1, P = 0.0255 |
| APACHEII | 17.0/23.0/29.8 | 18.0/23.0/30.0 | 17.0/23.0/31.0 | F = 0.22, DF = 1.244, P = 0.641 |
| SURGICAL | 26% (14) | 22% (42) | 23% (56) | X² = 0.39, DF = 1, P = 0.531 |
| SEP.ADMIT | 78% (42) | 79% (152) | 79% (194) | X² = 0.05, DF = 1, P = 0.825 |
| SEP.ANY | 85% (46) | 80% (154) | 81% (200) | X² = 0.69, DF = 1, P = 0.407 |
| SS.ADMIT | 52% (28) | 58% (112) | 57% (140) | X² = 0.72, DF = 1, P = 0.395 |
| SS.ANY | 63% (34) | 67% (128) | 66% (162) | X² = 0.26, DF = 1, P = 0.612 |

Asian subjects who had SIRS who carried the coagulation factor III 1826 A allele had lower survival than Asian subjects who had SIRS who carried the coagulation factor III 1826 G allele (survival: A=53%, G=67%, p=0.0765). Asian subjects who had SIRS who carried the coagulation factor III 1826 A allele had significantly more acute lung injury as reflected by the fewer days alive and free of acute lung injury (p=0.0533), more respiratory dysfunction as reflected by fewer days alive and free of respiratory dysfunction (p=0.0838) and significantly greater need for mechanical ventilation as reflected by fewer days alive and free of ventilation (p=0.0518) (TABLE 35). Asian subjects who had SIRS who carried the coagulation factor III 1826 A allele had more cardiovascular dysfunction as reflected by fewer days alive and free of vasopressors (p=0.092), and fewer days alive and free of cardiovascular dysfunction (0.0932) (TABLE 35). Asian subjects who had SIRS who carried the coagulation factor III 1826 A allele had significantly more severe systemic inflammatory response as reflected by fewer days alive and free of 4 of 4 SIRS criteria (p=0.0604) (TABLE 35). Asian subjects who had SIRS who carried the coagulation factor III 1826 A allele had significantly more need for steroid treatment as reflected by fewer days alive and free of steroids (p=0.0052) (TABLE 35). Thus Asian subjects who had SIRS who carried the coagulation factor III 1826 A allele had more acute lung injury, more respiratory dysfunction, more need for ventilation, more cardiovascular dysfunction and need for cardiovascular support, more severe systemic inflammatory response and more need for steroids (TABLE 35).

TABLE 35
Days alive and free (DAF) of organ dysfunction by 1826 A/G allele of tissue factor in
cohort of critically ill Asian subjects who had systematic inflammatory response
syndrome.

| | G | A | Combined | Test |
|---|---|---|---|---|
| | (N = 54) | (N = 192) | (N = 246) | Statistic |
| SURV | 67% (36) | 53% (102) | 56% (138) | X² = 3.14, DF = 1, P = 0.0765 |
| ALI.DAF | 6.5/26.5/28.0 | 2.0/13.0/28.0 | 2.0/13.0/28.0 | F = 3.77, DF = 1.244, P = 0.0533 |
| PRESS.DAF | 7.5/24.0/28.0 | 2.0/18.0/28.0 | 2.0/17.5/28.0 | F = 2.86, DF = 1.244, P = 0.092 |
| MSIRS4.DAF | 9.25/27.00/28.00 | 4.00/22.00/28.00 | 4.00/22.50/28.00 | F = 3.56, DF = 1.244, P = 0.0604 |
| STER.DAF | 6/20/2028 | 1/6/2028 | 1/7/2028 | F = 7.87, DF = 1.244, P = 0.00542 |
| CVS.DAF | 5.75/15.50/26.75 | 1.00/9.50/26.00 | 1.00/9.00/26.00 | F = 2.84, DF = 1.244, P = 0.0932 |
| RESP.DAF | 5.0/20.5/27.0 | 0.0/8.0/27.0 | 0.0/8.5/26.0 | F = 3.01, DF = 1.244, P = 0.0838 |
| VENT.DAF | 0.25/16.50/27.00 | 0.00/4.00/26.00 | 0.00/5.00/26.00 | F = 3.82, DF = 1.244, P = 0.0518 |

ii) Allele Analysis—Cohort of Asian Subjects Who Had Sepsis

Of the Asian who had sepsis, 194 were successfully genotyped for polymorphisms of coagulation factor III 1826 A/G and were included in this analysis. The frequency of the genotypes is shown in TABLE 36. These alleles were in Hardy Weinberg equilibrium in our population (TABLE 36). There were no significant differences in baseline characteristics of subjects according to the coagulation factor III 1826 A/G genotype (TABLE 36). Subjects had a similar distribution of age, gender, medical/surgical statues, APACHE II scores upon admission, septic shock upon admission and septic shock anytime.

TABLE 36
Baseline characteristics of cohort of critically ill Asian subjects who had sepsis for tissue
factor 1826 A/G defined by allele.

| | G | A | Combined | Test |
|---|---|---|---|---|
| | (N = 42) | (N = 152) | (N = 194) | Statistic |
| AGE | 65.3/73.0/76.0 | 54.0/68.0/76.0 | 56.8/68.0/76.0 | F = 2.76 DF = 1.192 P = 0.0985 |
| SEX | 81% (34) | 57% (86) | 62% (120) | X² = 8.29 DF = 1 P = 0.004 |
| APACHEII | 17.3/24.0/30.0 | 18.0/23.0/32.0 | 18.0/23.0/32.0 | F = 0.03 DF = 1.192 P = 0.872 |
| SURGICAL | 26% (11) | 24% (37) | 25% (48) | X² = 0.06 DF = 1 P = 0.806 |
| SS.ADMIT | 67% (28) | 74% (112) | 72% (140) | X² = 0.81 DF = 1 P = 0.369 |
| SS.ANY | 76% (32) | 83% (126) | 81% (158) | X² = 0.98 DF = 1 P = 0.323 |

Asian subjects who had sepsis who carried the coagulation factor III 1826 A allele had more acute lung injury as reflected by the fewer days alive and free of acute lung injury (p=0.0896). Asian subjects who had sepsis who carried the coagulation factor III 1826 A allele had greater need for steroid treatment as reflected by fewer days alive and free of steroids (p=0.0052) (TABLE 37). Asian subjects who had sepsis who carried the coagulation factor III 1826 A allele had significantly more cardiovascular dysfunction as reflected by fewer days alive and free of cardiovascular dysfunction (0.0973) (TABLE 37). Asian subjects who had sepsis who carried the coagulation factor III 1826 A allele had greater need for ventilation as shown by fewer days alive and free of mechanical ventilation (p=0.0889) (TABLE 37). Thus Asian subjects who had sepsis who carried the coagulation factor III 1826 A allele had, more acute lung injury, more cardiovascular dysfunction, a significantly greater need for steroids and more need for mechanical ventilation (TABLE 37).

TABLE 37
Days alive and free (DAF) of organ dysfunction by 1826 A/G allele of tissue factor in
cohort of critically ill Asian subjects who had sepsis.

| | G | A | Combined | Test |
|---|---|---|---|---|
| | (N = 42) | (N = 152) | (N = 194) | Statistic |
| ALI.DAF | 3.5/18.5/28.0 | 2.0/9.0/28.0 | 2.0/9.0/28.0 | F = 2.91, DF = 1.192, P = 0.0896 |
| STER.DAF | 6/13/2028 | 0/5/28 | 1/6/2028 | F = 7.99, DF = 1.192, P = 0.0052 |
| CVS.DAF | 4.0/13.0/26.0 | 1.0/9.0/25.0 | 1.0/8.0/23.8 | F = 2.78, DF = 1.192, P = 0.0973 |
| VENT.DAF | 0.25/11.50/26.00 | 0.00/1.50/24.00 | 0.00/3.00/23.75 | F = 2.92, DF = 1.192, P = 0.0889 |

iii) Recessive Analysis—Cohort of Asian Subjects Who Had SIRS

Of the Asian subjects who had SIRS, 123 were successfully genotyped for polymorphisms of coagulation factor III 1826 A/G and were included in this analysis. The frequency of the genotypes (AA/GA vs GG) is shown in TABLE 38. There were no significant differences in baseline characteristics of subjects who had SIRS according to the coagulation factor III 1826 AA/GA genotypes vs. the GG genotype (TABLE 38). Subjects had a similar distribution of age, gender, medical/surgical statues, APACHE II scores upon admission, sepsis upon admission, sepsis anytime, septic shock upon admission and septic shock anytime.

TABLE 38
Baseline characteristics of cohort of critically ill Asian subjects who had systematic
inflammatory response syndrome by genotype of tissue factor 1826 A/G (GG vs. AA/CA).

| | GG | AA/GA | Combined | Test |
|---|---|---|---|---|
| | (N = 7) | (N = 116) | (N = 123) | Statistic |
| AGE | 63.0/74.0/78.0 | 50.8/67.0/74.3 | 53.8/68.0/76.0 | F = 1.28, DF = 1.121, P = 0.26 |
| SEX | 71% (5) | 60% (70) | 61% (75) | X² = 0.34, DF = 1, P = 0.559 |
| APACHEII | 17.5/19.0/25.5 | 17.0/23.0/30.0 | 17.0/23.0/31.0 | F = 1.23, DF = 1.121, P = 0.269 |
| SURGICAL | 43% (3) | 22% (25) | 23% (28) | X² = 1.7, DF = 1, P = 0.192 |
| SEP.ADMIT | 86% (6) | 78% (91) | 79% (97) | X² = 0.21, DF = 1, P = 0.647 |
| SEP.ANY | 100% (7) | 80% (93) | 81% (100) | X² = 1.71, DF = 1, P = 0.191 |
| SS.ADMIT | 43% (3) | 58% (67) | 57% (70) | X² = 0.6, DF = 1, P = 0.439 |
| SS.ANY | 57% (4) | 66% (77) | 66% (81) | X² = 0.25, DF = 1, P = 0.617 |

Asian subjects were either AA or GA for the coagulation factor III 1826 SNP had significantly more acute lung injury as reflected by the fewer days alive and free of acute lung injury (p=0.0243), fewer days alive and free of respiratory dysfunction (p=0.079) and significantly fewer days alive and free of mechanical ventilation (p=0.0346) (TABLE 39) than subjects who were coagulation factor III 1826 GG. Asian subjects who had SIRS who were either AA or GA for the coagulation factor III 1826 SNP had significantly fewer days alive and free of vasopressors as reflected by the fewer days alive and free of vasopressors (p=0.0161) and had significantly fewer days alive and free of cardiovascular dysfunction (p=0.0277) than subjects who were coagulation factor III 1826 GG (TABLE 39). Asian subjects who had SIRS who were either AA or GA for the coagulation factor III 1826 SNP had a significantly more coagulation dysfunction as reflected by fewer days alive and free of coagulation dysfunction (p=0.0143), had significantly fewer days alive and free of acute hepatic (p=0.0351) and of any hepatic dysfunction (p=0.0340) than subjects who were coagulation factor III 1826 GG (TABLE 39). Asian subjects who had SIRS were either AA or GA for the coagulation factor III 1826 SNP had more severe SIRS as reflected by the fewer days alive and free of 4 of 4 SIRS criteria (p=0.0677) than subjects who were coagulation factor III 1826 GG (TABLE 39). Asian subjects who had SIRS who were either AA or GA for the coagulation factor III 1826 SNP had significantly greater need for steroids as shown by fewer days alive and free of steroids (p=0.0066) (TABLE 39). Thus subjects who were either AA or GA for the coagulation factor III 1826 SNP and had SIRS had more acute lung injury, more respiratory dysfunction, more need for ventilation, more cardiovascular dysfunction, greater need for vasopressors, more coagulation dysfunction, more acute hepatic dysfunction, more severe SIRS and more need for steroids.

TABLE 39
Days alive and free (DAF) of organ dysfunction by 1826 A/G genotype (GG vs. AA/GA)
of tissue factor in cohort of critically ill Asian subjects who had systematic inflammatory
response syndrome.

| | GG | AA/GA | Combined | Test |
|---|---|---|---|---|
| | (N = 7) | (N = 116) | (N = 123) | Statistic |
| ALI.DAF | 28/28/28 | 2/13/2028 | 2/13/2028 | F = 5.2, DF = 1.121, P = 0.0243 |
| PRESS.DAF | 27.0/28.0/28.0 | 2.0/17.5/28.0 | 2.0/17.5/28.0 | F = 5.96, DF = 1.121, P = 0.0161 |
| MSIRS4.DAF | 27.0/27.0/28.0 | 4.0/22.5/28.0 | 4.0/22.5/28.0 | F = 3.4, DF = 1.121, P = 0.0677 |
| STER.DAF | 28/28/28 | 1/7/2028 | 1/7/2028 | F = 7.64, DF = 1.121, P = 0.00659 |
| CVS.DAF | 22.0/26.0/27.0 | 1.0/9.5/26.0 | 1.0/9.0/26.0 | F = 4.97, DF = 1.121, P = 0.0277 |
| RESP.DAF | 24.0/27.0/27.0 | 0.0/8.5/27.0 | 0.0/8.5/26.0 | F = 3.14, DF = 1.121, P = 0.079 |
| VENT.DAF | 22.5/26.0/27.0 | 0.0/5.0/26.0 | 0.0/5.0/26.0 | F = 4.57, DF = 1.121, P = 0.0346 |
| COAG.DAF | 28.00/28.00/28.00 | 3.75/19.50/28.00 | 3.00/20.00/28.00 | F = 6.17, DF = 1.121, P = 0.0143 |
| ACHEP.DAF | 28.0/28.0/28.0 | 3.0/17.5/28.0 | 2.0/15.5/28.0 | F = 4.54, DF = 1.121, P = 0.0351 |
| ANYHEP.DAF | 28.0/28.0/28.0 | 2.0/17.5/28.0 | 2.0/15.5/28.0 | F = 4.6, DF = 1.121, P = 0.0340 |

iv) Recessive Analysis—Cohort of Asian Subjects Who Had Sepsis

Of the Asian subjects who had sepsis, 97 were successfully genotyped for polymorphisms of coagulation factor III 1826 A/G and were included in this analysis. The frequency of the genotypes (AA/GA vs. GG) is shown in TABLE 40. There were no significant differences in baseline characteristics of subjects who had sepsis according to the coagulation factor III 1826 AA/GA genotypes vs. the GG genotype (TABLE 40). Subjects had a similar distribution of age, gender, medical/surgical statues, APACHE II scores upon admission, septic shock upon admission and septic shock anytime.

TABLE 40
Baseline characteristics of cohort of critically ill Asian subjects who had sepsis by
genotype of tissue factor 1826 A/G (GG vs. AA/GA).

| | GG | AA/GA | Combined | Test |
|---|---|---|---|---|
| | (N = 6) | (N = 91) | (N = 97) | Statistic |
| AGE | 73.3/75.0/79.0 | 55.5/68.0/75.5 | 57.5/68.0/76.0 | F = 2.09, DF = 1.95, P = 0.151 |
| SEX | 83% (5) | 60% (55) | 62% (60) | X² = 1.25, DF = 1, P = 0.263 |
| APACHEII | 18.3/21.0/26.8 | 18.0/23.0/32.0 | 18.0/23.0/32.0 | F = 0.49, DF = 1.95, P = 0.484 |
| SURGICAL | 50% (3) | 23% (21) | 25% (24) | X² = 2.19, DF = 1, P = 0.139 |
| SS.ADMIT | 50% (3) | 74% (67) | 72% (70) | X² = 1.56, DF = 1, P = 0.211 |
| SS.ANY | 67% (4) | 82% (75) | 81% (79) | X² = 0.92, DF = 1, P = 0.336 |

Asian subjects who had sepsis who were either AA or GA for the coagulation factor III 1826 SNP had lower survival (p=0.097) than subjects who were coagulation factor III 1826 GG. Asian subjects who had sepsis who were either AA or GA for the coagulation factor III 1826 SNP had significantly more acute lung injury as reflected by the fewer days alive and free of acute lung injury (p=0.029), significantly fewer days alive and free of respiratory dysfunction (p=0.0604) and significantly fewer days alive and free of mechanical ventilation (p=0.083) (TABLE 41) than subjects who were coagulation factor III 1826 GG. Asian subjects who had sepsis who were either AA or GA for the coagulation factor III 1826 SNP had significantly fewer days alive and free of vasopressors as reflected by the fewer days alive and free of vasopressors (p=0.0143) and had significantly fewer days alive and free of cardiovascular dysfunction (p=0.0099) than subjects who were coagulation factor III 1826 AA (TABLE 41). Asian subjects who had sepsis who were either AA or GA for the coagulation factor III 1826 SNP had a significantly more coagulation dysfunction as reflected by fewer days alive and free of coagulation dysfunction (p=0.0158), and significantly fewer days alive and free of acute hepatic (p=0.0376) and of any hepatic dysfunction (p=0.0360) than subjects who were coagulation factor III 1826 GG (TABLE 41). Asian subjects who had sepsis who were either AA or GA for the coagulation factor III 1826 SNP had more neurological dysfunction as reflected by the fewer days alive and free of neurological dysfunction (p=0.0702) than subjects who were coagulation factor III 1826 GG (TABLE 41). Asian subjects who had sepsis who were either AA or GA for the coagulation factor III 1826 SNP had significantly more severe SIRS as reflected by the fewer days alive and free of 4 of 4 SIRS criteria (p=0.0627) than subjects who were coagulation factor III 1826 GG (TABLE 41). Asian subjects who had sepsis who were either AA or GA for the coagulation factor III 1826 SNP had significantly greater need for steroids as shown by fewer days alive and free of steroids (p=0.0093) (TABLE 41). Thus subjects who were either AA or GA for the coagulation factor III 1826 SNP and had sepsis had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 41
Days alive and free (DAF) of organ dysfunction by 1826 A/G genotype (GG vs. AA/GA)
of tissue factor in cohort of critically ill Asian subjects who had sepsis.

| | GG | AA/GA | Combined | Test |
|---|---|---|---|---|
| | (N = 6) | (N = 91) | (N = 97) | Statistic |
| SURV | 83% (5) | 48% (44) | 51% (49) | X² = 2.76, DF = 1, P = 0.097 |
| ALI.DAF | 28/28/28 | 2/9/2028 | 2/9/2028 | F = 4.92, DF = 1.95, P = 0.029 |
| PRESS.DAF | 26.5/28.0/28.0 | 2.0/12.0/26.5 | 2.0/12.0/27.0 | F = 6.03, DF = 1.95, P = 0.0159 |
| MSIRS4.DAF | 27.0/27.0/27.8 | 3.5/18.0/27.0 | 3.5/18.0/27.0 | F = 3.55, DF = 1.95, P = 0.0627 |
| STER.DAF | 28/28/28 | 1/6/2028 | 1/6/2028 | F = 7.05, DF = 1.95, P = 0.00931 |
| CVS.DAF | 26.0/26.5/27.0 | 1.0/9.0/24.5 | 1.0/8.0/23.5 | F = 6.92, DF = 1.95, P = 0.00992 |
| RESP.DAF | 23.0/26.5/27.0 | 0.0/6.0/24.0 | 0.0/6.0/24.5 | F = 3.61, DF = 1.95, P = 0.0604 |
| VENT.DAF | 20.8/26.0/26.8 | 0.0/3.0/23.5 | 0.0/3.0/23.5 | F = 4.96, DF = 1.95, P = 0.0283 |
| CNS.DAF | 24.8/27.5/28.0 | 3.5/18.0/28.0 | 3.0/18.0/28.0 | F = 3.35, DF = 1.95, P = 0.0702 |
| COAG.DAF | 28.0/28.0/28.0 | 3.5/16.0/28.0 | 3.0/16.0/28.0 | F = 6.04, DF = 1.95, P = 0.0158 |
| ACHEP.DAF | 28.0/28.0/28.0 | 2.5/13.0/28.0 | 2.0/12.0/28.0 | F = 4.45, DF = 1.95, P = 0.0376 |
| ANYHEP.DAF | 28/28/28 | 2/13/2028 | 2/12/2028 | F = 4.52, DF = 1.95, P = 0.0360 |

v) Recessive Analysis—Cohort of Asian Subjects Who Had Septic Shock

Of the Asian subjects who had septic shock, 70 were successfully genotyped for polymorphisms of coagulation factor III 1826 A/G and were included in this analysis. The frequency of the genotypes (AA/GA vs. GG) is shown in TABLE 42. There were no significant differences in baseline characteristics of subjects who had septic shock according to the coagulation factor III 1826 AA/GA genotypes vs. the GG genotype (TABLE 42). Subjects had a similar distribution of age, gender, medical/surgical statues and APACHE II scores upon admission.

TABLE 42
Baseline characteristics of cohort of critically ill Asian subjects who had septic shock by
genotype of tissue factor 1826 A/G (GG vs. AA/GA).

| | GG | AA/GA | Combined | Test |
|---|---|---|---|---|
| | (N = 3) | (N = 67) | (N = 70) | Statistic |
| AGE | 54.0/76.0/80.0 | 58.5/68.0/74.5 | 61.5/68.0/76.0 | F = 0.3, DF = 1.68, P = 0.585 |
| SEX | 67% (2) | 63% (42) | 63% (44) | X² = 0.02, DF = 1, P = 0.889 |
| APACHEII | 20.5/23.0/25.5 | 21.5/27.0/32.0 | 21.0/26.0/32.5 | F = 0.78, DF = 1.68, P = 0.38 |
| SURGICAL | 67% (2) | 21% (14) | 23% (16) | X² = 3.41, DF = 1, P = 0.0647 |

Asian subjects who had septic shock who were either AA or GA for the coagulation factor III 1826 SNP had lower survival (p=0.0685) than subjects who were coagulation factor III 1826 GG. Asian subjects who had septic shock who were either AA or GA for the coagulation factor III 1826 SNP had significantly more acute lung injury as reflected by the fewer days alive and free of acute lung injury (p=0.0292), significantly fewer days alive and free of respiratory dysfunction (p=0.0431) and significantly fewer days alive and free of mechanical ventilation (p=0.0238) (TABLE 43) than subjects who were coagulation factor III 1826 GG. Asian subjects who had septic shock who were either AA or GA for the coagulation factor III 1826 SNP had significantly fewer days alive and free of vasopressors as reflected by the fewer days alive and free of vasopressors (p=0.0234) and had significantly fewer days alive and free of cardiovascular dysfunction (p=0.0136) than subjects who were coagulation factor III 1826 GG (TABLE 43). Asian subjects who had septic shock who were either AA or GA for the coagulation factor III 1826 SNP had a significantly more coagulation dysfunction as reflected by fewer days alive and free of coagulation dysfunction (p=0.0265), and significantly fewer days alive and free of acute hepatic (p=0.044) and of any hepatic dysfunction (p=0.044) than subjects who were coagulation factor III 1826 GG (TABLE 43). Asian subjects who had septic shock who were either AA or GA for the coagulation factor III 1826 SNP had more neurological dysfunction as reflected by the fewer days alive and free of neurological dysfunction (p=0.0579) than subjects who were coagulation factor III 1826 GG (TABLE 43). Asian subjects who had septic shock who were either AA or GA for the coagulation factor III 1826 SNP had more severe SIRS as reflected by the fewer days alive and free of 4 of 4 SIRS criteria (p=0.0679) than subjects who were coagulation factor III 1826 GG (TABLE 43). Asian subjects who had septic shock who were either AA or GA for the coagulation factor III 1826 SNP had significantly greater need for steroids as shown by fewer days alive and free of steroids (p=0.0283) (TABLE 43). Thus subjects who were either AA or GA for the coagulation factor III 1826 SNP and had septic shock had more acute lung injury, respiratory dysfunction, more need for ventilation, more cardiovascular dysfunction, greater need for vasopressors, more coagulation dysfunction, more acute hepatic dysfunction, more neurological dysfunction more severe SIRS and more need for steroids.

TABLE 43
Days alive and free (DAF) of organ dysfunction by 1826 A/G genotype (GG vs. AA/GA)
of tissue factor in cohort of critically ill Asian subjects who had septic shock.

| | GG | AA/GA | Combined | Test |
|---|---|---|---|---|
| | (N = 3) | (N = 67) | (N = 70) | Statistic |
| SURV | 100% (3) | 46% (31) | 49% (34) | X² = 3.32, DF = 1, P = 0.0685 |
| ALI.DAF | 28/28/28 | 1/6/2028 | 1/6/2028 | F = 4.97, DF = 1.68, P = 0.0292 |
| PRESS.DAF | 27/28/28 | 1/12/2026 | 1/9/2026 | F = 5.38, DF = 1.68, P = 0.0234 |
| MSIRS4.DAF | 27.0/27.0/27.5 | 3.0/15.0/27.0 | 2.5/15.0/27.0 | F = 3.44, DF = 1.68, P = 0.0679 |
| STER.DAF | 28.0/28.0/28.0 | 0.5/5.0/28.0 | 1.0/5.0/28.0 | F = 5.02, DF = 1.68, P = 0.0283 |
| CVS.DAF | 26.0/26.0/26.5 | 0.0/4.0/23.0 | 0.0/4.0/22.5 | F = 6.42, DF = 1.68, P = 0.0136 |
| RESP.DAF | 26.5/27.0/27.0 | 0.0/3.0/23.5 | 0.0/1.0/24.0 | F = 4.25, DF = 1.68, P = 0.0431 |
| VENT.DAF | 26.5/27.0/27.0 | 0.0/1.0/22.0 | 0.0/1.0/22.5 | F = 5.35, DF = 1.68, P = 0.0238 |
| CNS.DAF | 27.5/28.0/28.0 | 3.0/15.0/27.5 | 3.0/15.0/27.5 | F = 3.72, DF = 1.68, P = 0.0579 |
| COAG.DAF | 28.0/28.0/28.0 | 3.0/13.0/27.5 | 2.0/13.0/28.0 | F = 5.14, DF = 1.68, P = 0.0265 |
| ACHEP.DAF | 28.0/28.0/28.0 | 2.0/11.0/28.0 | 1.5/9.0/28.0 | F = 4.21, DF = 1.68, P = 0.044 |
| ANYHEP.DAF | 28.0/28.0/28.0 | 2.0/11.0/28.0 | 1.5/9.0/28.0 | F = 4.21, DF = 1.68, P = 0.044 |

3.1.5 Coagulation factor III T13925C

i) Allele Analysis—Cohort of Caucasian Subjects Who Had SIRS

Of the Caucasian subjects who had SIRS, 832 were successfully genotyped for the 13925 polymorphism of coagulation factor III and included in this analysis. The frequencies of the genotypes are shown in TABLE 44. These genotypes are observed to be in Hardy Weinberg equilibrium. There are no significant differences observed in baseline characteristics of subjects in relation to coagulation factor III 13925 genotype (TABLE 44). Subjects had a similar distribution of age, gender, medical/surgical status, APACHE II scores upon admission, sepsis upon admission, sepsis anytime, septic shock upon admission and septic shock anytime.

TABLE 44
Baseline characteristics by coagulation factor III 13925 C/T alleles in critically ill
Caucasian subjects with SIRS

| | T | C | Combined | Test |
|---|---|---|---|---|
| | (N = 450) | (N = 1214) | (N = 1664) | Statistic |
| AGE | 44.0/58.0*70.0 | 45.3/59.0/71.0 | 46.0/59.0/71.0 | F = 0.38 d.f. = 1.1662 P = 0.538 |
| SEX\hat{ }2 | 63% (282) | 64% (772) | 63% (1054) | X\hat{ }2 = 0.12 d.f. = 1 P = 0.728 |
| APACHEII | 16.3/23.0/28.0 | 16.0/22.0/28.0 | 16.0/22.0/28.0 | F = 0.59 d.f. = 1.1662 P = 0.441 |
| SURGICAL | 23% (104) | 25% (302) | 24% (406) | X\hat{ }2 = 0.55 d.f. = 1 P = 0.456 |
| SEP.ADMIT | 80% (362) | 81% (980) | 81% (1342) | X\hat{ }2 = 0.02 d.f. = 1 P = 0.898 |
| SEP.ANY | 82% (370) | 83% (1004) | 83% (1374) | X\hat{ }2 = 0.05 d.f. = 1 P = 0.819 |
| SS.ADMIT | 52% (236) | 57% (686) | 55% (922) | X\hat{ }2 = 2.19 d.f. = 1 P = 0.139 |
| SS.ANY | 56% (252) | 61% (736) | 59% (988) | X\hat{ }2 = 2.91 d.f. = 1 P = 0.0879 |

Caucasian subjects who had SIRS and carried the T allele of coagulation factor III 13925 had significantly more acute lung injury as reflected by fewer days alive and free of acute lung injury (p=0.0502; TABLE 45). Caucasian subjects who had SIRS and carried the T allele of coagulation factor III 13925 had significantly greater need for steroids as reflected by fewer days alive and free of steroids (p=0.0888; TABLE 45). Caucasian subjects who had SIRS and carried the T allele of coagulation factor III 13925 had significantly more acute renal failure as demonstrated by fewer days alive free of acute renal failure (p=0.0518; TABLE 45). Furthermore, Caucasian subjects who had SIRS and carried the T allele of coagulation factor III 13925 had significantly more renal dysfunction as demonstrated by fewer days alive free of any renal dysfunction (p=0.0188; TABLE 45) subjects who had SIRS and carried the T allele of coagulation factor III 13925 had significantly greater need for renal support as shown by fewer days alive and free of renal support (p=0.0177; TABLE 45).

TABLE 45
Days alive and free (DAF) of organ dysfunction for coagulation factor III 13925 C/T
alleles in critically ill Caucasian subjects with SIRS.

| | T | C | Combined | Test |
|---|---|---|---|---|
| | (N = 450) | (N = 1214) | (N = 1664) | Statistic |
| SURV | 63% (283) | 66% (807) | 66% (1090) | X\hat{ }2 = 1.87 d.f. = 1 P = 0.172 |
| ALI.DAF | 2.25/18.50/28.00 | 4.00/21.00/28.00 | 3.00/21.00/28.00 | F = 3.84 d.f. = 1.1662 P = 0.0502 |
| STER.DAF | 1/11/28 | 2/20/28 | 1/17/28 | F = 5.76 d.f. = 1.1662 P = 0.0165 |
| CNS.DAF | 5/25/28 | 8/26/28 | 6/26/28 | F = 2.9 d.f. = 1.1662 P = 0.0888 |
| ACRF.DAF | 2.0/22.0/28.0 | 4.0/25.028.0 | 3.0/24.5/28.0 | F = 3.79 d.f. = 1.1662 P = 0.0518 |
| ANYREN.DAF | 1/17/28 | 3/22/28 | 2/21/28 | F = 5.53 d.f. = 1.1662 P = 0.0188 |
| RENSUP.DAF | 2.25/26.00/28.00 | 5.00/28.00/28.00 | 3.00/28.00/28.00 | F = 5.63 d.f. = 1.1662 P = 0.0177 |

ii) Allele Analysis-Cohort of Caucasian Subjects who had Septic Shock

Of the Caucasian subjects with septic shock, 461 were successfully genotyped for the 13925 polymorphism of coagulation factor III and included in this analysis. The frequencies of the genotypes are shown in TABLE 46. These genotypes are observed to be in Hardy Weinberg equilibrium. There are no significant differences observed in baseline characteristics of subjects in relation to coagulation factor 13925 genotype (TABLE 46). Subjects had a similar distribution of age, gender, medical/surgical status, APACHE II scores upon admission, sepsis upon admission, sepsis anytime, septic shock upon admission and septic shock anytime.

TABLE 46
Baseline characteristics by coagulation factor III 13925 C/T alleles in critically ill
Caucasian subjects with septic shock

| | T | C | Combined | Test |
|---|---|---|---|---|
| | (N = 236) | (N = 686) | (N = 922) | Statistic |
| AGE | 47/62/72 | 48/61/72 | 48/61/72 | F = 0.11 d.f. = 1.920 P = 0.745 |
| SEX\hat{ }2 | 62% (147) | 65% (445) | 64% (592) | X\hat{ }2 = 0.51 d.f. = 1 P = 0.476 |
| APACHEII | 20.8/26.0/32.0 | 20.0/26.0/31.0 | 20.0/26.0/32.0 | F = 0.01 d.f. = 1.920 P = 0.91 |
| SURGICAL | 25% (58) | 28% (192) | 27% (250) | X\hat{ }2 = 1.03 d.f. = 1 P = 0.309 |

Caucasian subjects who had septic shock and carried the A allele of coagulation factor III 13925 had a decreased 28-day survival rate compared to subjects who carried the coagulation factor III 13925 C allele (p=0.0867; TABLE 46). Similarly, Caucasian subjects who had septic shock and carried the T allele of coagulation factor III 13925 survived for significantly fewer days than those Caucasian subjects who had septic shock and carried the C allele (p=0.0627; TABLE 46). Caucasian subjects with septic shock who carried the T allele of coagulation factor III 13925 had greater cardiovascular dysfunction as demonstrated by days alive free of cardiovascular dysfunction (p=0.069; TABLE 47). Caucasian subjects who had septic shock and carried the T allele of coagulation factor III 13925 had significantly increased neurological dysfunction as evidenced by days alive free of neurological dysfunction (p=0.0504; TABLE 47). Caucasian subjects who had septic shock and carried the T allele of coagulation factor III 13925 were observed to have significantly increased acute renal failure as shown by days alive free of acute renal failure (p=0.0431; TABLE 47). Similarly, Caucasian subjects who had septic shock and carried the T allele of coagulation factor III 13925 had significantly more renal dysfunction as demonstrated by fewer days alive free of any renal dysfunction (p=0.00745; TABLE 47). Furthermore, Caucasian subjects who had septic shock and carried the T allele of coagulation factor III 13925 had a significantly greater need for renal support as shown by days alive free of renal support (p-0.00389; TABLE 47).

TABLE 47
Days alive and free (DAF) of organ dysfunction for coagulation factor III 13925 C/T
alleles in critically ill Caucasian subjects with septic shock.

| | T | C | Combined | Test |
|---|---|---|---|---|
| | (N = 236) | (N = 686) | (N = 922) | Statistic |
| SURV | 53% (126) | 60% (410) | 58% (536) | X\hat{ }2 = 2.93 d.f. = 1 P = 0.0867 |
| DA | 7/28/28 | 9/28/28 | 8/28/28 | F = 3.47 d.f. = 1.920 P = 0.0627 |
| STER.DAF | 1.0/6.0/28.0 | 2.0/14.5/28.0 | 1.0/11.0/28.0 | F = 6.01 d.f. = 1.920 P = 0.0144 |
| CVS.DAF | 0.0/13.0/23.0 | 2.0/15.0/24.0 | 0.0/13.5/24.0 | F = 3.31 d.f. = 1.920 P = 0.069 |
| CNS.DAF | 5.0/20.5/27.3 | 6.0/24.0/28.0 | 5.0/23.0/28.0 | F = 3.84 d.f. = 1.920 P = 0.0504 |
| ACRF.DAF | 1/14/28 | 3/20/28 | 3/18/28 | F = 4.1 d.f. = 1.920 P = 0.0431 |
| ANYREN.DAF | 0/9/28 | 2/18/28 | 2/15/28 | F = 7.19 d.f. = 1.920 P = 0.00745 |
| RENSUP.DAF | 1.75/15.00/28.00 | 3.00/24.00/28.00 | 3.00/21.00/28.00 | F = 8.38 d.f. = 1.920 P = 0.00389 |

### 3.2 Association of Coagulation Factor III (F3) Haplotypes with Improved Response to Therapy

Therapies for sepsis, SIRS and septic shock may include mechanical ventilation, support of circulation with vasopressors and inotropic agents, antibiotics, drainage of abscesses and surgery as appropriate. Activated protein C (APC or XIGRIS™ (when referring to APC as sold by Eli Lilly & Co., Indianapolis Ind.)) can improve survival of subjects having sepsis, SIRS and septic shock. The PROWESS trial (BERNARD G R. et al. New Eng. J. Med. (2001) 344:699-709)) showed that XIGRIS™ decreased 28-day mortality from 31% to (placebo) to 25% (APC/XIGRIS™—treated). XIGRIS™ was particularly effective in subjects at high risk of death for example as identified by having an APACHE II score greater than or equal to 25. XIGRIS™ has been approved for treatment of severe sepsis at increased risk of death. In some jurisdictions, the high risk of death is identified as having an APACHE II score greater than or equal to 25; in other jurisdictions high risk of death is identified as having 2 or more organ dysfunctions or having an APACHE II score greater than or equal to 25.

### General Methods

### Cohort Description

All patients admitted to the ICU of St. Paul's Hospital (Vancouver, BC, Canada) were screened for inclusion. The ICU is a mixed medical-surgical ICU in a tertiary care, university-affiliated teaching hospital. Severe sepsis was defined as the presence of at least two systemic inflammatory response syndrome criteria and a known or suspected source of infection plus at least one new organ dysfunction by Brussels criteria (at least moderate, severe or extreme). From this cohort we identified XIGRIS™-treated subjects who were critically ill patients who had severe sepsis, no XIGRIS™ contraindications (e.g. platelet count >30,000, International normalization ration (INR)<3.0) and were treated with XIGRIS™. Control subjects were critically ill patients who had severe sepsis (at least 2 of 4 SIRS criteria, known or suspected infection, and APACHE II≧25), a platelet count>30,000, INR<3.0, bilirubin<20 mmol/L and were not treated with XIGRIS™. Accordingly, the control group (untreated with XIGRIS™) is comparable to the XIGRIS™-treated group.

### Genotyping

F3 A1826G and G1089A were genotyped using the TaqMan™ assay (Applied Biosystems) as described above.

### Clinical Phenotype

Our primary outcome variable was 28-day mortality. Secondary outcome variables were organ dysfunctions. Baseline demographics recorded were age, gender, admission APACHE II score (KNAUS W A. et al. Crit. Care Med (1985) 13:818-829), and medical or surgical diagnosis on admission to the ICU (based on the APACHE III diagnostic codes) (KNAUS W A. et al. Chest (1991) 100:1619-1636). After meeting the inclusion criteria, data were recorded for each 24-hour period (8 am to 8 am) for 28-days after ICU admission or until hospital discharge to evaluate organ dysfunction and the intensity of SIRS (Systemic Inflammatory Response Syndrome) and sepsis. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value and missing data after day one was substituted by carrying forward the previous day's value. When data collection for each patient was complete, all patient identifiers were removed from all records and the patient file was assigned a unique random number linked with the blood samples. The completed raw data file was used to calculate descriptive and severity of illness scores using standard definitions as described below.

Baseline characteristics key.

| Baseline | |
|---|---|
| Characteristic | Description |
| AGE | Age, in years |
| SEX/GENDER | % Male |
| APACHE II | APACHE II score |
| SURGICAL | % Surgical admissions |
| SS.ADMIT | % Patients with septic shock upon admission |
| SS.ANY | % Patients with septic shock anytime during admission |

Secondary outcome variables key.

Secondary Outcome Description

Day alive and free of cardiovascular dysfunction
Days alive and free of use of vasopressors
Days alive and free of inotropic agents
Days alive and free of acute lung injury
Days alive and free of respiratory dysfunction
Days alive and free of use of mechanical ventilators
Days alive and free of acute renal dysfunction
Days alive and free of any of renal dysfunction
Days alive and free of renal support
Days alive and free of coagulation dysfunction
Days alive and free of INR >1.5
Days alive and free of neurological dysfunction
Days alive and free of acute hepatic dysfunction
Days alive and free of ¾ SIRS criteria

Organ dysfunction was evaluated at baseline and daily using the Brussels score (SIBBALD W J. and VINCENT J L. Chest (1995) 107(2):522-7) (TABLE 2A). If the Brussels score was moderate, severe, or extreme dysfunction then organ dysfunction was recorded as present on that day. To correct for deaths during the observation period, we calculated the days alive and free of organ dysfunction (RUSSELL J A. et al. Crit. Care Med (2000) 28(10):3405-11 and BERNARD G R. et al. Chest (1997) 112(1):164-72). For example, the severity of cardiovascular dysfunction was assessed by measuring days alive and free of cardiovascular dysfunction over a 28-day observation period. Days alive and free of cardiovascular dysfunction was calculated as the number of days after inclusion that a patient was alive and free of cardiovascular dysfunction over 28-days. Thus, a lower score for days alive and free of cardiovascular dysfunction indicates more cardiovascular dysfunction. The reason that days alive and free of cardiovascular dysfunction is preferable to simply presence or absence of cardiovascular dysfunction is that severe sepsis has a high acute mortality so that early death (within 28-days) precludes calculation of the presence or absence of cardiovascular dysfunction in dead patients. Organ dysfunction has been evaluated in this way in observational studies [34] and in randomized controlled trials of new therapy in sepsis, acute respiratory distress syndrome (BERNARD G R. et al. N Engl J Med (1997) 336(13):912-8) and in critical care (HEBERT P C. et al. N Engl J Med (1999) 340(6):409-17).

To further evaluate cardiovascular, respiratory, and renal function we also recorded, during each 24 hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis).

We also scored the presence of three or four of the SIRS criteria each day over the 28-day observation period as a cumulative measure of the severity of SIRS. SIRS was considered present when subjects met at least two of four SIRS criteria. The SIRS criteria were 1) fever (>38° C.) or hypothermia (<35.5° C.), 2) tachycardia (>100 beats/min in the absence of beta blockers, 3) tachypnea (>20 breaths/min) or need for mechanical ventilation, and 4) leukocytosis (total leukocyte count >11,000/μL).

### Statistical Analysis

Baseline characteristics age, gender, APACHE II, and percent surgical patients were recorded in both groups and compared using a chi-squared or Kruskal-Wallis test where appropriate. For each SNP of F3 the 28-day survival rate (%) for patients who were treated with XIGRIS™ was compared to control patients who were not treated with XIGRIS™ using a chi-squared test. We considered a by-genotype effect to be significant when two criteria were fulfilled. First, we required an increase of ≧15% in 28-day survival rate in the XIGRIS™ treated group compared to the control group. Second, we required that p<0.1 for this comparison. When both criteria were met we considered the polymorphism allele or genotype which predicted increased 28-day survival with XIGRIS™ treatment to be an “Improved Response Polymorphism” (IRP). Organ dysfunction were compared between XIGRIS™-treated patients and matched controls using a Kruskal-Wallis test.

### Kaplan-Meier Methods

Kaplan-Meier 28-day survival curves were constructed using the Survival package in R to compare patients who were treated with XIGRIS™ to the matched controls (patients who were not treated with XIGRIS™) within each of the following groups: (1) FIII 1826 AA/AG; (2) FIII 1826 GG; (3) FIII 1089 G; and (4) FIII 1089 A.

### Baseline Characteristics

Baseline characteristics for the XIGRIS™-treated patients (N=49) and the matched controls (N=250) are given in TABLE 48. These are typical of subjects who have severe sepsis with regards to age, sex and APACHE II score.

TABLE 48
Baseline characteristics (Age, Gender, % Surgical, APACHE II) for XIGRIS ™-treated
patients matched control patients (not treated with XIGRIS ™). Data are shown as 25
percentile/median/75 percentile. Statistical analysis was conducted using a chi-squared or
Kruskal-Wallis test (F) where appropriate. D.F., degrees of freedom.

| | Matched | | | | | |
|---|---|---|---|---|---|---|
| | Controls | XIGRIS ™-Treated | TOTAL | | | |
| | (N = 250) | Patients (N = 49) | (N = 299) | Test Statistic | D.F. | P-VALUE |
| AGE | 51/63/73 | 38/52/67 | 49/62/72 | F = 10.45 | 1, 297 | 0.00137 |
| SEX | 65% (163) | 57% (28) | 64% (191) | Chisquare = 1.15 | 1 | 0.283 |
| APACHEII | 27/29/33.75 | 23/32/37 | 26/29/34 | F = 0.18 | 1, 297 | 0.674 |
| SURGICAL | 22% (55) | 29% (14) | 23% (69) | Chisquare = 1.0 | 1 | 0.318 |
| SS.ADMIT | 83% (208) | 90% (44) | 84% (252) | Chisquare = 1.35 | 1 | 0.246 |
| SS.ANY | 88% (219) | 92% (45) | 88% (264) | Chisquare = 0.71 | 1 | 0.399 |

### 3.2.1 Coagulation Factor III (F3) A1826G and Improved Response to Therapy with Activated Protein C (XIGRIS™)

### Sample Size

There were 42 patients who were genotyped for F3 A1826G who were treated with XIGRIS™ and 215 control patients (not treated with XIGRIS™) who were genotyped for F3 A1826G. Among the XIGRIS™-treated patients (N=42), there were 10 patients with the F3 1826 GG genotype and 32 patients with the F3 1826 AG/AA genotypes. Among the control patients (not treated with XIGRIS™) (N=215), there were 42 patients with the F3 1826 GG genotype and 173 patients with F3 1826 AG/AA genotypes.

### Survival

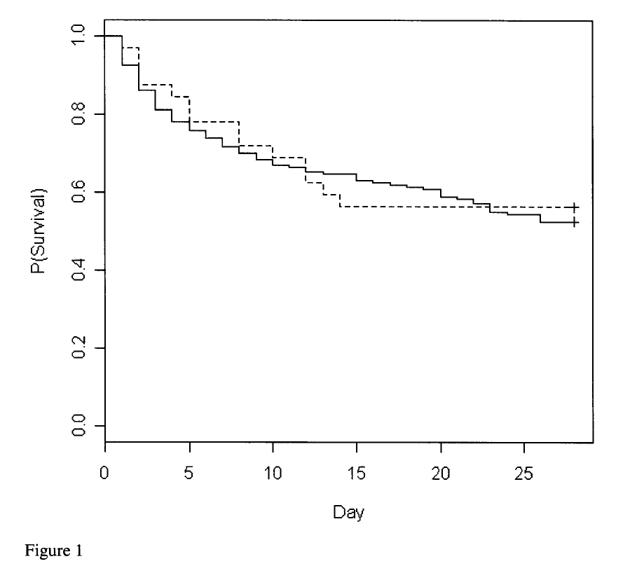

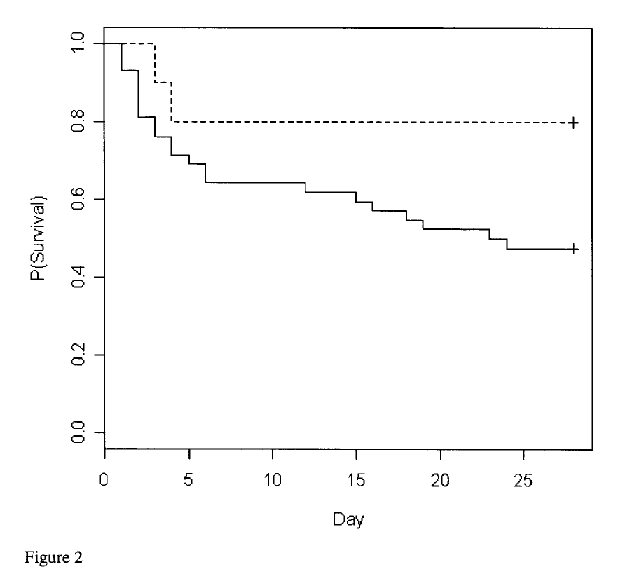

Patients who were IRP positive (i.e. F3 1826 GG) who were treated with XIGRIS™ had a much higher survival (80%, Column B, Table 49) than did patients who were IRP positive (i.e. F3 1826 GG) who did not receive XIGRIS™ (48%, p=0.0649; IRP Matched Controls, Column A, Table 49) (see FIGS. 1 and 2).

TABLE 49
28-day survival of XIGRIS ™-treated patients and matched controls (patients
not treated with XIGRIS ™) by genotype at F3 A1826G in a cohort of critically ill patients
who had severe sepsis and no XIGRIS ™ contraindications. Data is presented for both
improved response polymorphism (IRP) and non-IRP patients. The chi square tests and
the reported P-values correspond to the comparison of IRP Matched Controls to IRP
XIGRIS ™-treated patients only (Column A versus Column B). 28-day survival is given
as % survival (N survived/N total). D.F., degrees of freedom.

| | | 28-Day Survival | | | | | | |
|---|---|---|---|---|---|---|---|---|
| | | A | B | C | D | | | |
| | | IRP | IRP | non-IRP | non-IRP | A vs B | | |
| | | Matched | XIGRIS ™- | Matched | XIGRIS ™- | Chi- | | P- |
| SNP | IRP | Controls | Treated | Controls | Treated | square | D.F. | VALUE |
| 1826 | GG | 48% (20/42) | 80% (8/10) | 53% (91/173) | 56% (18/32) | 3.41 | 1 | 0.0649 |

### Organ Dysfunctions of IRP Patients Compared to Those of Non-IRP Patients

Organ dysfunctions were also compared between IRP patients and patients having genotypes other than the IRP at F3 A1826G (TABLE 50). Results are reported as the difference in median days alive and free of a given organ dysfunction between both (1) IRP patients and non-IRP patients in the matched-control group and (2) IRP XIGRIS™-treated patients and non-IRP XIGRIS™-treated patients. The average difference in days alive and free of different organ dysfunctions in XIGRIS™-treated patients is greater than the difference in matched controls. Furthermore, the IRP patients have fewer days alive and free than the non-IRP patients when they are not treated with XIGRIS™.

In contrast and of note, the IRP positive patients (i.e. F3 1826 GG) have more days alive and free of organ dysfunction when treated with XIGRIS™ (Column C, Table 50) than do the IRP positive patients (i.e. F3 1826 GG) when they are not treated with XIGRIS™ (Column A, Table 50). This confirms that the IRP genotype identifies patients who are IRP positive (i.e. F3 1826 GG) who respond particularly well to XIGRIS™.

TABLE 50
Organ dysfunction scored as median days alive and free (DAF) of organ dysfunction of
matched controls (patients who were not treated with XIGRIS ™) and patients who were
treated with XIGRIS ™ according to F3 A1826G (GG vs AG/AA).

| | TREATMENT | | | | | |
|---|---|---|---|---|---|---|
| | NO XIGRIS ™ | | | XIGRIS ™ | | |
| | GENOTYPE | | | | | |
| | GG | AG/AA | | GG | AG/AA | |
| | (N = 42) | (N = 173) | Δ | (N = 10) | (N = 32) | Δ |
| | A | B | A − B | C | D | C − D |
| CVS.DAF | 8.5 | 14 | −5.5 | 24.5 | 14 | 10.5 |
| RESP.DAF | 2.5 | 6 | −3.5 | 22.5 | 4 | 18.5 |
| ACRF.DAF | 12.5 | 14 | −1.5 | 20.5 | 16.5 | 4 |
| CNS.DAF | 17 | 18 | −1 | 24.5 | 22.5 | 2 |
| COAG.DAF | 15 | 23 | −8 | 27.5 | 17 | 10.5 |
| ACHEP.DAF | 23.5 | 28 | −4.5 | 28 | 16.5 | 11.5 |
| MSIRS3.DAF | 5.5 | 9 | −3.5 | 22 | 2 | 20 |
| PRESS.DAF | 16.5 | 19 | −2.5 | 24.5 | 17 | 7.5 |
| INO.DAF | 23.5 | 26 | −2.5 | 27 | 26.5 | 0.5 |
| VENT.DAF | 0 | 3 | −3 | 22 | 3 | 19 |
| RENSUP.DAF | 15.5 | 13 | 2.5 | 27 | 9.5 | 17.5 |
| ANYREN.DAF | 8.5 | 9 | −0.5 | 20.5 | 14 | 6.5 |
| INR.DAF | 18 | 23 | −5 | 28 | 26 | 2 |
| AVERAGE | 12.8 | 15.8 | −3.0 | 24.5 | 14.5 | 10.0 |

### 3.2.2 Coagulation Factor III (F3) G1089A and Improved Response to Therapy with Activated Protein C (XIGRIS™)

### Sample Size

There were 43 patients who were genotyped for F3 G1089A who were treated with XIGRIS™ and 214 control patients (not treated with XIGRIS™) who were genotyped for F3 G1089A. Among the XIGRIS™-treated patients (N=43), there were 47 F3 1089 G alleles and 39 F3 1089 A alleles. Among the control patients (not treated with XIGRIS™) (N=214), there were 244 F3 1089 G alleles and 184 F3 1089 A alleles.

### Survival

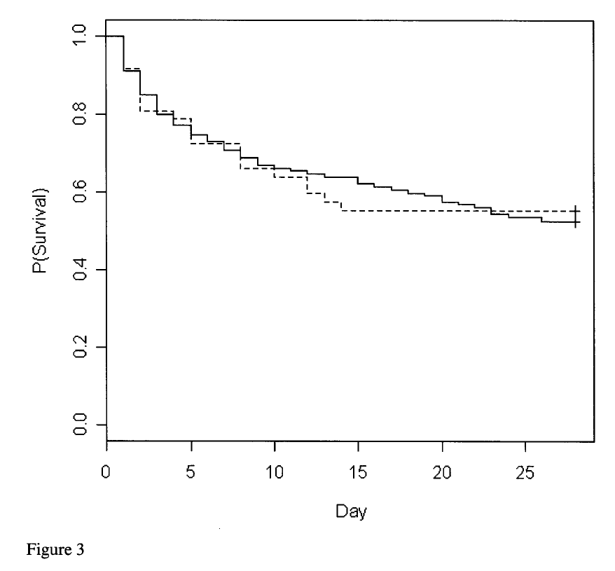

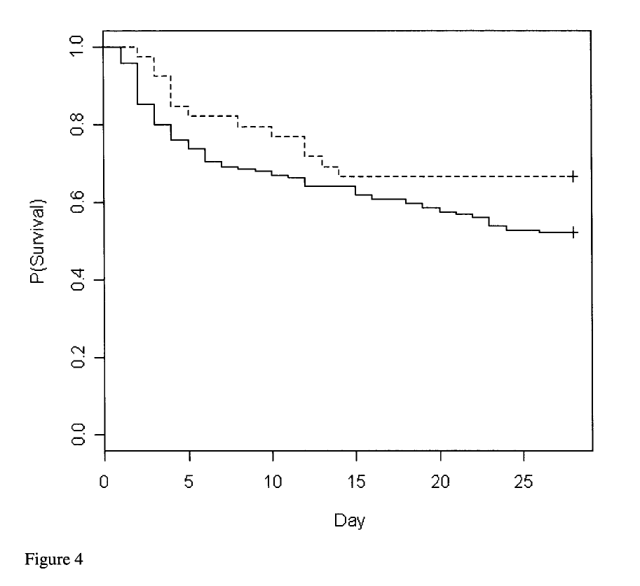

Patients who were IRP positive (i.e. F3 1089 A) who were treated with Xigris had a much higher survival (67%, Column B, Table 49) than did patients who were IRP positive (i.e. F3 1089 A) who did not receive Xigris (52%, p=0.0636; IRP Matched Controls, Column A, Table 49) (see FIGS. 3 and 4).

TABLE 51
28-day survival of XIGRIS ™-treated patients and matched controls (patients not treated
with XIGRIS ™) by allele at F3 G1089A in a cohort of critically ill patients who had
severe sepsis and no XIGRIS ™ contraindications. Data is presented for both improved
response polymorphism (IRP) and non-IRP patients. The chi square tests and the reported
P-values correspond to the comparison of IRP Matched Controls to IRP XIGRIS ™-treated
patients only (Column A versus Column B). 28-day survival is given as % survival (N
alleles survived/N alleles total). D.F., degrees of freedom.

| | | 28-Day Survival | | | | | | |
|---|---|---|---|---|---|---|---|---|
| | | | B | | D | | | |
| | | A | IRP | C | non-IRP | | | |
| | | IRP | XIGRIS ™- | non-IRP | XIGRIS ™- | A vs B | | |
| | | Matched | Treated | Matched | Treated | Chi- | | P- |
| SNP | IRP | Controls | Patients | Controls | Patients | square | D.F. | VALUE |
| 1089 | A | 52% | 67% | 52% | 55% | 3.48 | 1 | 0.0636 |
| | | (96/184) | (26/39) | (128/244) | (26/47) | | | |

Organ Dysfunctions of IRP Patients Compared to those of Non-IRP Patients

Organ dysfunctions were also compared between IRP positive ((i.e. F3 1089 A) patients and patients having alleles/genotypes other than the IRP (TABLE 52) for F3 G1089A. Results are reported as the difference in median days alive and free of a given organ dysfunction between both (1) IRP patients and non-IRP patients in the matched-control group and (2) IRP XIGRIS™-treated patients and non-IRP XIGRIS™-treated patients. The average difference in days alive and free of different organ dysfunctions in XIGRIS™-treated patients is greater than the difference in matched controls. Furthermore, the IRP patients have fewer days alive and free than the non-IRP patients when they are not treated with XIGRIS™.

In contrast and of note, the IRP positive patients (i.e. F3 1089 A) have more days alive and free of organ dysfunction when treated with Xigris (Column C, Table 52) than do the IRP positive patients (i.e. (i.e. F3 1089 A)) when they are not treated with XIGRIS™ (Column A, Table 52). This confirms that the IRP allele identifies patients who are IRP positive (i.e. F3 1089 A) who respond particularly well to XIGRIS™.

TABLE 52
Organ dysfunction scored as median days alive and free (DAF) of organ dysfunction of
matched controls (patients who were not treated with XIGRIS ™) and patients who were
treated with XIGRIS ™ according to F3 1089 allele (A vs G).

| | TREATMENT | | | | | |
|---|---|---|---|---|---|---|
| | NO XIGRIS ™ | | | XIGRIS ™ | | |
| | ALLELE | | | | | |
| | A | G | | A | G | |
| | (N = 184) | (N = 244) | Δ | (N = 39) | (N = 47) | Δ |
| | A | B | A − B | C | D | C − D |
| ACHEP.DAF | 28 | 27 | 1 | 28 | 19 | 9 |
| ACRF.DAF | 14.5 | 12 | 2.5 | 19 | 19 | 0 |
| ANYREN.DAF | 11 | 6.5 | 4.5 | 15 | 14 | 1 |
| CNS.DAF | 18 | 17 | 1 | 23 | 23 | 0 |
| COAG.DAF | 21 | 23 | −2 | 24 | 20 | 4 |
| CVS.DAF | 13.5 | 14 | −0.5 | 15 | 15 | 0 |
| INO.DAF | 25 | 26 | −1 | 28 | 28 | 0 |
| INR.DAF | 21 | 22.5 | −1.5 | 28 | 26 | 2 |
| MSIRS3.DAF | 8.5 | 9 | −0.5 | 7 | 2 | 5 |
| PRESS.DAF | 19 | 18 | 1 | 24 | 17 | 7 |
| RENSUP.DAF | 15 | 9 | 6 | 25 | 10 | 15 |
| RESP.DAF | 4 | 4.5 | −0.5 | 17 | 4 | 13 |
| VENT.DAF | 2 | 1 | 1 | 17 | 3 | 14 |
| AVERAGE | 15.4 | 14.6 | 0.9 | 20.8 | 15.4 | 5.4 |

### 3.3 Association of Factor III Haplotypes with Increased Hypertension

### Methods

### Cardiac Surgery Cohort

The coagulation factor III (F3) G13925A SNP (or rs3354 T/C) was studied in an independent Caucasian cohort (N=102) of subjects scheduled for first time elective coronary artery bypass grafting that required cardiopulmonary bypass. We refer to this independent non-septic, SIRS cohort as the Cardiac Surgery cohort. The Cardiac Surgery cohort was reviewed for significant associations between the coagulation factor III G13925A SNP and the occurrence of hypertension

The Institutional Review Board at Providence Health Care and the University of British Columbia approved this study.

### Study Inclusion Criteria

In the cohort of non-septic SIRS subjects who had cardiopulmonary bypass surgery, individuals were included in the analysis if they met diagnostic criteria for SIRS. Subjects were excluded from the study if they had undergone 1) urgent or emergency cardiopulmonary bypass surgery or 2) valve or repeat cardiac surgery. Subjects who had urgent or emergency cardiopulmonary by pass surgery were excluded because they may have had an inflammatory response due to other triggers (i.e. shock). Subjects who had valve surgery or repeat cardiac surgery were excluded due to the likelihood that they possess different pre-operative pathophysiology or experience longer total surgical and cardiopulmonary bypass time than subjects having elective cardiopulmonary bypass surgery.

### Clinical Phenotypes

After meeting the inclusion criteria, phenotypic data were recorded for subjects at 24-hour intervals (8 am to 8 am) for 28 days post-ICU admission or until hospital discharge to evaluate organ dysfunction and the intensity of SIRS and sepsis. We recorded age, sex, whether patients were current smokers, whether patients had diabetes meleitus and whether patients had hypertension prior to surgery. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value, and missing data after day one was substituted by carrying forward the previous day's value.

When data collection for each patient was complete, patient identifiers were removed from all records, and the patient file was assigned a unique random number linked to its respective blood sample. The completed raw data file was used to calculate descriptive and severity of illness scores using the standard definitions described below.

### Measurement of Chemokines and Cytokines

After induction of anesthesia and placement of systemic and pulmonary artery catheters that were routinely inserted for clinical purposes at our institution, blood was obtained prior to cardiopulmonary bypass for baseline measurement (0 hours) of serum GCSF and again at 3 hours post-surgery.

### SNP Selection

Identification and annotation of the coagulation factor III G13925A SNP was undertaken as discussed in the general methods section preceding the examples. The coagulation factor III G13925A SNP is located in the 3′ UTR of the coagulation factor III gene and thus may play a role in mRNA stability or mRNA processing (STRACHAN and REID, 2004).

### Genotyping

Discarded whole blood samples, stored at 4° C., were collected from the hospital laboratory. The buffy coat was extracted and the samples were transferred to 1.5 mL cryotubes, barcoded and cross-referenced with the unique patient number, and stored at −80° C. DNA was extracted from the buffy coat using a QIA amp DNA maxi kit (Qiagen, Mississauga, ON, Canada). Enrolled ICU subjects were genotyped using the 5′ nuclease, Taqmamm (Applied Biosystems; Foster City, Calif.) polymerase chain reaction (PCR) method.

### Data Analysis

The primary outcome variables for the cardiac surgery cohort was the induction of hypertension. All data analysis was carried out using statistical packages available in R (R Core Development Group, 2005—R Development Core Team (www.R-project.org). Vienna Austria 2005). Chi-squared and Kruskal-Wallis test statistics were used in conjunction with Cox proportional hazards (CPH) regression to identify significant SNP-phenotype and haplotype-phenotype associations, as well as to identify baseline characteristics that may require post-hoc, multivariate adjustment. SNP analysis was carried out comparing allele vs. phenotype. Haplotype-phenotype analyses were carried out using chi-squared statistics and the score statistics of SCHAID D J. et al. Hum Hered. (2003) 55(2-3):86-96.

### Legend

Hypertension Key

| | |
|---|---|
| H.TENSE | Hypertensive |
| EJEC.FRAC | Ejection Fraction |
| BYPASS | Bypass Time |
| CLAMP | Clamp Time |
| APROTININ | Aprotinin Use |
| GCSF | Granulocyte Colony Stimulating Factor |

Note.
X/X/X = 25%-ile/median/75%-ile

### Results

### 3.3.1 Coagulation Factor III G13925A

Table 53 summarizes the baseline characteristics (i.e., age, gender, smoker, diabetes, hypertension, preoperative ejection fraction, bypass time, cross-clamp time, and aprotinin use) of 68 non-septic SIRS subjects who were successfully genotyped for coagulation factor III G13925A. There were no significant differences in age, sex, smoker status, presence of diabetes, ejection reaction, bypass time, clamp time or use of aprototinin. There was a significant difference at baseline in hypertension.

TABLE 53
Baseline characteristics of a cohort of non-septic SIRS CSICU
subjects by coagulation factor III G13925A allele

| Clamp | A. Mean | A. Med | A. SD | G. Mean | G. Med | G. SD |
|---|---|---|---|---|---|---|
| Age | 65.1 | 66 | 8.97 | 65.8 | 65 | 7.97 |
| Sex | 0.579 | 1 | 0.5 | 0.694 | 1 | 0.463 |
| Smoker | 0.289 | 0 | 0.46 | 0.235 | 0 | 0.426 |
| Diabetes | 0.132 | 0 | 0.343 | 0.235 | 0 | 0.426 |
| H. Tense | 0.684 | 1 | 0.471 | 0.49 | 0 | 0.502 |
| Ejec. frac | 0.515 | 0.5 | 0.134 | 0.514 | 0.5 | 0.13 |
| Bypass | 1.76 | 1.73 | 0.606 | 1.77 | 1.63 | 0.653 |
| Clamp | 1.35 | 1.43 | 0.537 | 1.35 | 1.27 | 0.551 |
| Aprotinin | 0.1053 | 0 | 0.311 | 0.0612 | 0 | 0.241 |

Table 54 summarizes observed important SNP-biomarker associations. Subjects who have the coagulation factor III 13925 A allele were also observed to have an increased incidence of hypertension compared to subjects with the coagulation factor III 13925 G allele (p=0.0411). This finding suggests that the coagulation factor III 13925 SNP may affect the relationship between coagulation factor III expression in SIRS and/or septic shock and the development of hypertension.

TABLE 54
Association of coagulation factor III G13925A with hypertension
in a cohort of non-septic CSICU subjects diagnosed
with systematic inflammatory response syndrome.

| | A | G | Combined | Test |
|---|---|---|---|---|
| | (N = 38) | (N = 98) | (N = 136) | Statistic |
| H.Tense | 68% (26) | 49% (48) | 54% (74) | X\hat{ }2 = 4.17 d.f. = 1 |
| | | | | P = 0.0411 |

3.4 Biological Plausability

### Methods

### Biological Plausibility Cohort

The F3 G13925A (or rs3354 T/C) SNP was studied in an independent Caucasian cohort (N=102) of subjects scheduled for first time elective coronary artery bypass grafting that required cardiopulmonary bypass. This cohort, known as the “Biological Plausibility” (BP) cohort was reviewed for significant SNP-biomarker associations, which may provide useful insights into the cellular processes underlying the population-based SNP-phenotype associations localized in the Caucasian and Asian septic shock cohorts. On the basis that the F3 gene polymorphism is shown herein to be associated with altered survival and organ dysfunction, it was expected that such polymorphisms should also be associated with altered serum Granulocyte Colony Stimulating Factor (GCSF) because GCSF is a potent pro-inflammatory chemokine and altered IL-8 levels because IL-8 is a pro-inflammatory cytokine

The Institutional Review Board at Providence Health Care and the University of British Columbia approved this study.

### Measurement of Chemokines and Cytokines

After induction of anesthesia and placement of systemic and pulmonary artery catheters that were routinely inserted for clinical purposes at our institution, blood was obtained prior to cardiopulmonary bypass for baseline measurement (0 hours) of serum GCSF and again at 3 hours post-surgery.

### SNP Selection

Identification and annotation of the coagulation factor III G13925A SNP was undertaken as discussed in the general methods section preceding the examples. The coagulation factor III G13925A SNP is located in the 3′ UTR of the coagulation factor III gene and thus may play a role in mRNA stability or mRNA processing (Strachan and Reid, 2004).

### Genotyping

Discarded whole blood samples, stored at 4° C., were collected from the hospital laboratory. The buffy coat was extracted and the samples were transferred to 1.5 mL cryotubes, barcoded and cross-referenced with the unique patient number, and stored at −80° C. DNA was extracted from the buffy coat using a QIA amp DNA maxi kit (Qiagen, Mississauga, ON, Canada). Enrolled ICU subjects were genotyped using the 5′ nuclease, Taqman™ (Applied Biosystems; Foster City, Calif.) polymerase chain reaction (PCR) method.

### Data Analysis

The primary outcome variables for the biological plausibility cohort were change in post-operative GCSF from 0 hours pre-operatively to 3 hours post-surgery. All data analysis was carried out using statistical packages available in R(R Core Development Group, 2005—R Development Core Team (www.R-project.org). Vienna Austria 2005). Chi-squared and Kruskal-Wallis test statistics were used in conjunction with Cox proportional hazards (CPH) regression to identify significant SNP-phenotype and haplotype-phenotype associations, as well as to identify baseline characteristics that may require post-hoc, multivariate adjustment. SNP analysis was carried out comparing allele vs. phenotype. Haplotype-phenotype analyses were carried out using chi-squared statistics and the score statistics of Schaid (2003). We analyzed each cohort separately to avoid potential false positive associations caused by population stratification (Simpson's paradox) of a genetically mixed cohort.

### Legend

Biological Plausibility Key

| | |
|---|---|
| H.TENSE | Hypertensive |
| EJEC.FRAC | Ejection Fraction |
| BYPASS | Bypass Time |
| CLAMP | Clamp Time |
| APROTININ | Aprotinin Use |
| GCSF | Granulocyte Colony Stimulating Factor |

Note.
X/X/X = 25%-ile/median/75%-ile

### Results

### 3.4.1 Coagulation Factor III G13925A

Table 53 summarizes the baseline characteristics (i.e., age, gender, smoker, diabetes, hypertension, preoperative ejection fraction, bypass time, cross-clamp time, and aprotinin use) of 68 non-septic SIRS subjects who were successfully genotyped for coagulation factor III G13925A. No significant differences were detected at baseline accept hypertension (see 3.3; Table 53).

Table 55 summarizes observed important SNP-biomarker associations. Subjects who had the coagulation factor III 13925 A allele were observed to have a smaller increase in serum granulocyte colony stimulating factor (GCSF) levels post-cardiopulmonary bypass surgery than subjects with the coagulation factor III 13925 G allele (p=0.0842). This finding suggests that non-septic SIRS subjects who had the coagulation factor III 13925 A allele are more likely to experience a less intense GCSF response after cardiopulmonary bypass surgery.

TABLE 55
Association of coagulation factor III G13925A with GCSF
in a cohort of non-septic CSICU subjects
who had systematic inflammatory response syndrome.

| | A | G | Combined | Test |
|---|---|---|---|---|
| | (N = 38) | (N = 98) | (N = 136) | Statistic |
| GCSF.DIF | 148/212/301 | 182/273/417 | 161/240/372 | F = 3.03 |
| | | | | d.f. = 1.134 |
| | | | | P = 0.0842 |

Although the foregoing examples have been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for obtaining a prognosis for a subject having, or at risk of developing, an inflammatory condition, the method comprising determining a genotype of said subject which includes one or more polymorphic sites in the subject's coagulation factor III (F3) sequence, wherein said genotype is indicative of an ability of the subject to recover from the inflammatory condition and wherein the polymorphic site is one or more of the following: rs958587; rs3761955; rs1361600; rs696619; and rs3354; or one or more polymorphic sites in linkage disequilibrium therewith.

2. (canceled)

3. The method of claim 1, wherein the one or more polymorphic sites in linkage disequilibrium is selected from one or more of the polymorphic sites listed in TABLE 1B.

4. The method of claim 3, wherein the polymorphic site in linkage disequilibrium with one or more of rs958587, rs3761955, rs1361600, rs696619, and rs3354 has an r² value that is ≧0.8.

5-6. (canceled)

7. The method of claim 1, wherein said determining of genotype is achieved using nucleic acid sample from the subject.

8. (canceled)

9. The method claim 1, wherein said determining of genotype is done using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

10. (canceled)

11. The method of claim 1, wherein
(a) the subject is critically ill; and
(b) a risk allele is indicative of a prognosis of severe cardiovascular, respiratory, neurological, coagulation, hepatic or renal dysfunction and
(c) a protective allele is indicative of a prognosis of less severe cardiovascular respiratory neurological coagulation hepatic or renal dysfunction.

12. The method of claim 11, wherein the risk allele is one or more of the following:_rs958587C; rs3761955G; rs1361600A; rs696619C; and rs3354T; or one or more polymorphic sites in linkage disequilibrium therewith as listed in TABLE 1B and
wherein the protective allele is rs958587T; rs3761955A; rs1361600G; rs696619T; rs3354C; or one or more polymorphic sites in linkage disequilibrium therewith as listed in TABLE 1B.

13-15. (canceled)

16. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, a reaction to an ingested, inhaled, infused, injected, or delivered substance, glomerulonephritis, bowel infection, an opportunistic infections, an inflammatory response due to major surgery transplant or dialysis leading to an immunocompromised state, treatment with an immunosuppressive agent, HIV/AIDS, endocarditis, fever cystic fibrosis, diabetes mellitus, chronic renal failure, bronchiectasis, chronic obstructive lung disease, chronic bronchitis, emphysema, asthma, febrile neutropenia, meningitis, septic arthritis, urinary tract infection, necrotizing fasciitis, Group A streptococcus infection, splenectomy, recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystis carinii, pneumonia, Leishmaniasis, hemolytic uremic syndrome/thrombotic thrombocytopenic purpura, Dengue hemorrhagic fever, pelvic inflammatory disease, Legionella infection, Lyme disease, Influenza A infection, Epstein-Barr virus infection, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, graft-versus-host disease, transplant rejection, sickle cell anemia, nephrotic syndrome, toxicity of OKT3 therapy or cytokine therapy, and cirrhosis.

17. The method of claim 1, wherein the inflammatory condition is SIRS, sepsis and septic shock.

18-22. (canceled)

23. A kit for determining a genotype at a defined nucleotide position within a polymorphic site in a F3 sequence, wherein knowledge of the genotype provides a prognosis of the subject's ability to recover from an inflammatory condition, the kit comprising:
(a) a restriction enzyme capable of distinguishing alternate nucleotides at the polymorphic site; or
(b) a labeled oligonucleotides or peptide nucleic acid that is sufficiently complementary to an alternate nucleotide sequence at the polymorphic site so as to be capable of specifically hybridizing to said alternate nucleotide sequence, whereby the genotype of the polymorphic site may be determined; and
(c) optionally, instructions for use in determining the genotype
wherein the polymorphic site is selected from one or more of the following: rs958587; rs3761955; rs1361600; rs696619; and rs3354; or one or more polymorphic sites in linkage disequilibrium therewith.

24-26. (canceled)

27. A method for selecting a group of subjects for determining the efficacy of a candidate drug known or suspected of being useful for the treatment of an inflammatory condition, the method comprising
(a) determining a genotype for one or more polymorphic sites in a F3 sequence in each subject, wherein said genotype is indicative of the subject's ability to recover from the inflammatory condition and
(b) sorting subjects based on their genotype.

28. The method of claim 27 further comprising, administering the candidate drug to the subjects or a subset of subjects and determining each subject's ability to recover from the inflammatory condition.

29. The method of claim 28, further comprising comparing subject response to the candidate drug based on genotype of the subject.

30. Two or more nucleic acid molecules or peptide nucleic acid molecules of about 10 to about 400 nucleotides in length that hybridize specifically to
(a) a sequence contained in a human target sequence that consists of one or more of SEQ ID NO:1-SEQ ID NO:17,
(b) a complementary sequence of the target sequence; or
(c) an RNA equivalent of the target sequence;
wherein the molecules are operable in determining a polymorphism genotype.

31. (canceled)

32. Two or more nucleic acid molecules or peptide nucleic acid molecules selected from the group of oligonucleotide or peptide-nucleic acid probes consisting of:
(a) a probe that hybridizes under high stringency conditions to a nucleic acid molecule including SEQ ID NO:17 having a C at position 599 but not to a nucleic acid molecule including SEQ ID NO:17 having a T at position 599;
(b) a probe that hybridizes under high stringency conditions to a nucleic acid molecule including SEQ ID NO:17 having a T at position 599 but not to a nucleic acid molecule including SEQ ID NO:17 having a C at position 599;
(c) a probe that hybridizes under high stringency conditions to a nucleic acid molecule including SEQ ID NO:17 having a C at position 1089 but not to a nucleic acid molecule including SEQ ID NO:17 having a T at position 1089;
(d) a probe that hybridizes under high stringency conditions to a nucleic acid molecule including SEQ ID NO:17 having a T at position 1089 but not to a nucleic acid molecule including SEQ ID NO:17 having a C at position 1089;
(e) a probe that hybridizes under high stringency conditions to a nucleic acid molecule including SEQ ID NO:17 having a G at position 1826 but not to a nucleic acid molecule including SEQ ID NO:17 having an A at position 1826;
(f) a probe that hybridizes under high stringency conditions to a nucleic acid molecule including SEQ ID NO:17 having an A at position 1826 but not to a nucleic acid molecule including SEQ ID NO:17 having a G at position 1826;
(g) a probe that hybridizes under high stringency conditions to a nucleic acid molecule including SEQ ID NO:17 having a C at position 4524 but not to a nucleic acid molecule including SEQ ID NO:17 having a T at position 4524;
(h) a probe that hybridizes under high stringency conditions to a nucleic acid molecule including SEQ ID NO:17 having a T at position 4524 but not to a nucleic acid molecule including SEQ ID NO:17 having a C at position 4524;
(i) a probe that hybridizes under high stringency conditions to a nucleic acid molecule including SEQ ID NO:17 having a G at position 13925 but not to a nucleic acid molecule including SEQ ID NO:17 having an A at position 13925; and
(j) a probe that hybridizes under high stringency conditions to a nucleic acid molecule including SEQ ID NO: 17 having an A at position 13925 but not to a nucleic acid molecule including SEQ ID NO:17 having a G at position 13925.

33. An array comprising two or more oligonucleotide or peptide nucleic acid molecules according to claim 32.

34-49. (canceled)

50. A method of treating an inflammatory condition in an at risk subject, the method comprising:
(a) identifying a subject having a risk genotype in his F3 sequence; and
(b) administering an anti-inflammatory agent or an anti-coagulant agent to the subject.

51-52. (canceled)

53. The method of claim 50, further comprising determining the subject's APACHE II score as an assessment of subject risk.

54. The method of claim 50, further comprising determining the number of organ system failures for the subject as an assessment of subject risk.

55. The method of claim 53, wherein the subject's APACHE II score is indicative of an increased risk when ≧25.

56. The method of claim 54, wherein 2 or more organ system failures are indicative of increased subject risk.

57. The method of claim 50, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, a reaction to an ingested, inhaled, infused, injected, or delivered substance, glomerulonephritis, bowel infection, an opportunistic infections, an inflammatory response due to major surgery transplant or dialysis leading to an immunocompromised state, treatment with on an immunosuppressive agent, HIV/AIDS, endocarditis, fever, cystic fibrosis, diabetes mellitus, chronic renal failure, bronchiectasis, chronic obstructive lung disease, chronic bronchitis, emphysema, asthma, febrile neutropenia, meningitis, septic arthritis, urinary tract infection, necrotizing fasciitis, Group A streptococcus infection, splenectomy, recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, pulmonary embolism and venous thrombosis, mycobacterial tuberculosis, Pneumocystis carinii, pneumonia, Leishmaniasis, hemolytic uremic syndrome/thrombotic thrombocytopenic purpura, Dengue hemorrhagic fever, pelvic inflammatory disease, Legionella infection, Lyme disease, Influenza A infection, Epstein-Barr virus infection, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, graft-versus-host disease, transplant rejection, sickle cell anemia, nephrotic syndrome, toxicity of OKT3 therapy or cytokine therapy, and cirrhosis.

58. The method of claim 57, wherein the inflammatory condition is one or more of SIRS, sepsis and septic shock.

59. The method of claim 50, wherein the risk genotype is rs3761955G, rs1361600A or a polymorphic site in linkage disequilibrium therewith as set out in TABLE 1B.

60. (canceled)

61. The method of claim 59, wherein the subject having a risk allele is preferentially selected for administration one or more of the anti-inflammatory agents or the anti-coagulant agents:
(a) activated protein C;
(b) a tissue factor pathway inhibitors;
(c) platelet activating factor hydrolase;
(d) a PAF-AH enzyme analogue;
(e) an antibody to tumor necrosis factor alpha;
(f) soluble tumor necrosis factor receptor-immunoglobulin G1;
(g) procysteine;
(h) an elastase inhibitor;
(i) a human recombinant interleukin 1 receptor antagonists; and
(j) an antibody, inhibitor or antagonist to endotoxin, tumor necrosis factor receptor interleukin-6, high mobility group box, tissue plasminogen activator, bradykinin, CD-14, F3, Factor VII, Factor X or interleukin-10.

62-63. (canceled)

64. The method of claim 61, wherein the anti-inflammatory agent or the anti-coagulant agent is drotecogin alfa activated.

65. The method of claim 61, wherein the anti-inflammatory agent or the anti-coagulant agent is a monoclonal antibody to F3.

66. A method for obtaining a prognosis for a subject having, or at risk of developing, hypertension, the method comprising determining a genotype of said subject which includes one or more polymorphic sites in the subject's coagulation factor III (F3) sequence, wherein said genotype is indicative of the subject's likelihood of developing hypertension, wherein the polymorphic site is rs3354; or one or more polymorphic sites in linkage disequilibrium therewith.

67-74. (canceled)

75. The method of claim 66, wherein rs3354; or one or more polymorphic sites in linkage disequilibrium selected from rs841696A, rs3917628C, rs3917629TG, and rs841691A is predictive of an increased risk of hypertension.

76. (canceled)

77. The method of claim 66, wherein rs3354C or one or more polymorphic sites in linkage disequilibrium selected from rs841696G, rs3917628-, rs3917629-, and rs841691C is predictive of a decreased risk of hypertension.
